SEQUENCE LISTING (38 sequences)

SEQ ID NO: 1 - protein, 1296 aa, Clostridial botulinum A
  DOMAIN (1)...(448): Light chain, enzymatic domain; therapeutic domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - DNA, 3891 bp, Clostridial botulinum A
  misc_feature (1)...(1344): Nucleic acid region encoding Light chain; enzyme domain; protease domain; endopeptidase domain.
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 3 - protein, 1296 aa, Clostridia botulinum A (H227Y)
  SITE (227)...(227): H227Y mutatin that inactivates the endopeptidase activity of BoNT/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 4 - DNA, 3891 bp, Clostridia botulinum A (H227Y)
  mutation (678)...(680): The CAT code for Histidine 227 is changed to TAT and codes for Tyrosine.
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatatatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 5 - protein, 848 aa, Clostridium botulinum A H domain
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 6 - DNA, 2562 bp, Clostridium botulinum A H domain
aaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 7 - protein, 436 aa, Clostridium botulinum A Hc domain
  DOMAIN (1)...(436): Corresponds to amino acids 861-1296 of full length BoNT/A; Carboxy-terminal half of heavy chain; binding domain; acceptor binding domain; receptor binding domain; cell binding domain; targeting domain
RLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 8 - DNA, 1311 bp, Clostridium botulinum A Hc domain
  misc_feature (1)...(1311): Nucleic acid composition encoding carboxy-terminal half of heavy chain; binding domain; acceptor binding domain; receptor binding domain; cell binding domain; targeting domain. Corresponds to nucleotides 2581-3888 of full length BoNT/A;
agattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 9 - protein, 6 aa, Artificial Sequence
  A polyhistidine peptide
HHHHHH

SEQ ID NO: 10 - DNA, 18 bp, Artificial Sequence
  Nucleic acid compositon encoding a polyhistidine peptide
catcatcatcatcatcat

SEQ ID NO: 11 - protein, 808 aa, Homo sapiens FGFR3IIIb
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 12 - DNA, 2427 bp, Homo sapiens FGFR3IIIb
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 13 - protein, 806 aa, Homo sapiens FGFR3IIIc
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 14 - DNA, 2421 bp, Homo sapiens FGFR3IIIc
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 15 - protein, 694 aa, Homo sapiens FGFR3IIIS
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 16 - DNA, 2085 bp, Homo sapiens FGFR3IIIS
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 17 - protein, 802 aa, Bos taurus FGFR3IIIc
MGAPARALAFCVAVAVMTGAALGSPGVEPRVARRAAEVPGPEPSPQERAFGSGDTVELSCRLPAGVPTEPTVWVKDGVGLAPSDRVLVGPQRLQVLNASHEDAGAYSCRQRLSQRLLCLFSVRVTDAPSSGDDEGGDDEAEDTAGAPYWTRPERMDKKLLAVPAANTVRFRCPAAGNPTPSITWLKNGKEFRGEHRIGGIKLRQQQWSLVMESVVPSDRGNYTCVVENKFGRIQQTYTLDVLERSPHRPILQAGLPANQTAVLGSDVEFHCKVYSDAQPHIQWLKHVEVNGSKVGPDGTPYVTVLKTAGANTTDKELEVLSLRNVTFEDAGEYTCLAGNSIGFSHHSAWLVVLPAEEELVEAGEAGGVFAGVLSYGLGFLLFILAVAAVTLYRLRSPPKKGLGSPAVHKVSRFPLKRQVSLESSSSMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREYLRARRPPGTDYSFDTCRLPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSVPFEQYSPGGQDTPSSGSSGDDSVFAHDLLPPAPSGSGGSRT

SEQ ID NO: 18 - DNA, 2409 bp, Bos taurus FGFR3IIIc
atgggcgccccggctcgcgccctcgcgttttgcgtggcagtggcggtcatgaccggcgccgccctcgggtccccgggcgtggagccccgcgtcgcgcggagagcggcagaggtcccgggccccgagcccagcccgcaggagcgggcctttggcagcggggacaccgtggagctgagctgccgcttgccggcgggggtgcccacagagcccaccgtctgggtgaaggacggcgtgggcctggcgccctcggaccgcgtcctggtggggccgcagcggctacaggtgctcaacgcctcccacgaggacgccggagcctacagctgccgccagcgcctctcccagcggctgctgtgcctcttcagcgtgcgcgtgacagatgctccgtcctcaggggatgacgagggtggggacgacgaggccgaggacacagctggggccccttactggacgcggcctgagcggatggacaagaagctgctagcggtgccggccgccaacacggttcgcttccgctgcccagctgctggcaaccccacgccatccatcacctggctgaagaacggcaaggagttccggggcgagcaccgcatcgggggaatcaaactgcggcagcagcagtggagcctggtcatggagagcgtggtgccctcggaccgcggcaactacacgtgcgtcgtggagaacaagttcggcagaatccagcagacctacaccctggacgtgctggagcgctctccgcaccggcccatcctacaggccgggctgcccgctaaccagacagccgtgctgggcagcgatgtggagttccactgcaaggtctacagcgacgcccagccccacatccagtggctcaagcacgtggaggtgaacggcagcaaggtggggcccgacggcacgccctacgtcaccgtgctcaagacggcgggcgctaacaccaccgacaaggagctagaggttctatccttgcgcaatgtcacctttgaggacgcgggggagtacacatgtctggcgggcaattctatcgggttttcccatcactctgcgtggctggtggtgctgccagctgaggaggagctggtggaagccggtgaggctggcggtgtgttcgcgggtgtcctcagctacgggctgggcttcctcctcttcatcctggccgtggccgccgttacgctctaccgcctgaggagcccccctaagaagggcctgggctcgcccgcggtgcacaaggtctcccgcttcccgctcaagcgacaggtgtccttggagtccagctcatccatgagctccaacacaccgctggtacgcattgcccggctgtcatcgggcgagggccccaccctggccaacgtctctgagctcgagctgcccgccgaccccaagtgggagctgtcccgggcccggctgaccctgggcaagcctcttggggagggctgcttcggccaggtggtcatggcagaggccattggcatcgacaaggaccgagctgccaagcctgtcacggtggccgtgaagatgctgaaagatgacgccacggataaggacttatcggacctggtgtccgagatggagatgatgaagatgatcggaaaacacaagaacattatcaacctgctaggcgcctgcacgcagggcgggcccctgtacgtgctggtggagtacgcggccaagggcaacctgcgggaatacctgcgggcacggcggcccccgggcactgactactccttcgacacctgccggctgcccgaggagcagctcaccttcaaagacctggtgtcctgcgcctaccaggtggcgcggggcatggagtacctggcctcgcagaagtgcatccacagggacctggcggcccgcaacgtgctggtgactgaggacaacgtgatgaaaatcgccgacttcggcctggctcgtgacgtgcacaacctcgactactacaaaaagaccacaaacggccgcctgcccgtgaagtggatggcacccgaggccttgtttgaccgcgtctacacccaccaaagtgacgtctggtccttcggggtcctgctctgggagatcttcacgctggggggctcgccgtaccccggcatccccgtggaggagctcttcaagctgctgaaggaaggccaccgcatggacaagccggccaactgcacgcatgacctgtacatgatcatgcgcgagtgctggcacgccgcgccctcgcagaggcccaccttcaagcagctggtggaggacctggaccgtgtgctcaccgtgacgtccaccgacgagtacctggacctgtcggtgcccttcgagcagtactcgccgggcggccaggacacccccagctccggctcctcgggggacgactccgtgttcgctcacgacctgctgcccccggccccatccggcagcggaggctcgcggacgtga

SEQ ID NO: 19 - protein, 802 aa, Mus musculus FGFR3IIIb
MVVPACVLVFCVAVVAGATSEPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKSWISENVEADARLRLANVSERDGGEYLCRATNFIGVAEKAFWLRVHGPQAAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 20 - DNA, 2409 bp, Mus musculus FGFR3IIIb
atggtagtcccggcctgcgtgctagtgttctgcgtggcggtcgtggctggagctacttccgagcctcctggtccagagcagcgagttgtgcggagagcggcagaggttccagggcctgaacctagccagcaggagcaggtggccttcggcagtggggacaccgtggagctgagctgccatcctcctggaggtgcccccacagggcccacggtctgggctaaggatggtacaggtctggtggcctcccaccgcatcctggtggggcctcagaggctgcaagtgctaaatgcctcccacgaagatgcaggggtctacagctgccagcaccggctcactcggcgtgtgctgtgccacttcagtgtgcgtgtaacagatgctccatcctcaggagatgacgaagatggggaggacgtggctgaagacacaggggctccttattggactcgcccggagcgaatggataagaaactgctggctgtgccagccgcaaacactgtccgcttccgctgcccagctgctggcaaccctaccccctccatctcctggctgaagaatggcaaagaattccgaggggagcatcgcattgggggcatcaagctccggcaccagcagtggagcttggtcatggaaagtgtggtaccctccgatcgtggcaactatacctgtgtagttgagaacaagtttggcagcatccggcagacatacacactggatgtgctggagcgctccccacaccggcccatcctgcaggctgggctgccggccaaccagacagccattctaggcagtgacgtggagttccactgcaaggtgtacagcgatgcacagccacacatccagtggctgaagcacgtggaagtgaacggcagcaaggtgggccctgacggcacgccctacgtcactgtactcaagtcctggatcagtgagaatgtggaggcagacgcacgcctccgcctggccaatgtgtcggagcgggacgggggcgagtacctctgtcgagccaccaatttcataggcgtggctgagaaggccttttggctgcgtgttcacgggccccaagcagctgaggaggagctgatggaaactgatgaggctggcagcgtgtacgcaggcgtcctcagctacggggtggtcttcttcctcttcatcctggtggtggcagctgtgatactctgccgcctgcgcagtcccccaaagaagggcttgggctcgcccaccgtgcacaaggtctctcgcttcccgcttaagcgacaggtgtccttggaatctaactcctctatgaactccaacacaccccttgtccggattgcccggctgtcctcaggagaaggtcctgttctggccaatgtttctgaacttgagctgcctgctgaccccaagtgggagctatccaggacccggctgacacttggtaagcctcttggagaaggctgctttggacaggtggtcatggcagaagctattggcatcgacaaggaccgtactgccaagcctgtcaccgtggccgtgaagatgctgaaagatgatgcgactgacaaggacctgtcggacctggtatctgagatggagatgatgaaaatgattggcaagcacaagaacatcattaacctgctgggggcgtgcacacagggtgggcccctgtatgtgctggtggagtacgcagccaagggcaatctccgggagttccttcgggcgcggcggcctccaggcatggactactcctttgatgcctgcaggctgccagaggaacagctcacctgcaaggatctagtgtcctgtgcctaccaggtggcacggggcatggaatacttggcttctcagaagtgtattcacagagacttggctgccagaaacgtcctggtgaccgaggacaatgtgatgaagattgcggactttggcctggctcgagatgtgcacaacctggactactacaagaagaccacaaatggccggctacctgtgaagtggatggcaccagaggccctttttgaccgagtctacacccaccagagtgatgtttggtcttttggtgtcctcctctgggagatctttacgctggggggctcaccgtatcctggcatcccagtggaagagcttttcaagctgttgaaagagggccaccgcatggacaagccagccagctgcacacatgacctgtacatgatcatgcgggaatgttggcatgcggtgccttcacagaggcccaccttcaagcagttggtagaggatttagaccgcatcctcactgtgacatcaaccgacgagtacttggacctctccgtgccgtttgagcagtactcgccaggtggccaggacacgcctagctccagctcgtccggagatgactcggtgttcacccatgacctgctacccccaggtccacccagtaacgggggacctcggacgtga

SEQ ID NO: 21 - protein, 800 aa, Mus musculus FGFR3IIIc
MVVPACVLVFCVAVVAGATSEPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 22 - DNA, 2403 bp, Mus musculus FGFR3IIIc
atggtagtcccggcctgcgtgctagtgttctgcgtggcggtcgtggctggagctacttccgagcctcctggtccagagcagcgagttgtgcggagagcggcagaggttccagggcctgaacctagccagcaggagcaggtggccttcggcagtggggacaccgtggagctgagctgccatcctcctggaggtgcccccacagggcccacggtctgggctaaggatggtacaggtctggtggcctcccaccgcatcctggtggggcctcagaggctgcaagtgctaaatgcctcccacgaagatgcaggggtctacagctgccagcaccggctcactcggcgtgtgctgtgccacttcagtgtgcgtgtaacagatgctccatcctcaggagatgacgaagatggggaggacgtggctgaagacacaggggctccttattggactcgcccggagcgaatggataagaaactgctggctgtgccagccgcaaacactgtccgcttccgctgcccagctgctggcaaccctaccccctccatctcctggctgaagaatggcaaagaattccgaggggagcatcgcattgggggcatcaagctccggcaccagcagtggagcttggtcatggaaagtgtggtaccctccgatcgtggcaactatacctgtgtagttgagaacaagtttggcagcatccggcagacatacacactggatgtgctggagcgctccccacaccggcccatcctgcaggctgggctgccggccaaccagacagccattctaggcagtgacgtggagttccactgcaaggtgtacagcgatgcacagccacacatccagtggctgaagcacgtggaagtgaacggcagcaaggtgggccctgacggcacgccctacgtcactgtactcaagactgcaggcgctaacaccaccgacaaggagctagaggttctgtccttgcacaatgtcacctttgaggacgcgggggagtacacctgcctggcgggcaattctattgggttttcccatcactctgcgtggctggtggtgctgccagctgaggaggagctgatggaaactgatgaggctggcagcgtgtacgcaggcgtcctcagctacggggtggtcttcttcctcttcatcctggtggtggcagctgtgatactctgccgcctgcgcagtcccccaaagaagggcttgggctcgcccaccgtgcacaaggtctctcgcttcccgcttaagcgacaggtgtccttggaatctaactcctctatgaactccaacacaccccttgtccggattgcccggctgtcctcaggagaaggtcctgttctggccaatgtttctgaacttgagctgcctgctgaccccaagtgggagctatccaggacccggctgacacttggtaagcctcttggagaaggctgctttggacaggtggtcatggcagaagctattggcatcgacaaggaccgtactgccaagcctgtcaccgtggccgtgaagatgctgaaagatgatgcgactgacaaggacctgtcggacctggtatctgagatggagatgatgaaaatgattggcaagcacaagaacatcattaacctgctgggggcgtgcacacagggtgggcccctgtatgtgctggtggagtacgcagccaagggcaatctccgggagttccttcgggcgcggcggcctccaggcatggactactcctttgatgcctgcaggctgccagaggaacagctcacctgcaaggatctagtgtcctgtgcctaccaggtggcacggggcatggaatacttggcttctcagaagtgtattcacagagacttggctgccagaaacgtcctggtgaccgaggacaatgtgatgaagattgcggactttggcctggctcgagatgtgcacaacctggactactacaagaagaccacaaatggccggctacctgtgaagtggatggcaccagaggccctttttgaccgagtctacacccaccagagtgatgtttggtcttttggtgtcctcctctgggagatctttacgctggggggctcaccgtatcctggcatcccagtggaagagcttttcaagctgttgaaagagggccaccgcatggacaagccagccagctgcacacatgacctgtacatgatcatgcgggaatgttggcatgcggtgccttcacagaggcccaccttcaagcagttggtagaggatttagaccgcatcctcactgtgacatcaaccgacgagtacttggacctctccgtgccgtttgagcagtactcgccaggtggccaggacacgcctagctccagctcgtccggagatgactcggtgttcacccatgacctgctacccccaggtccacccagtaacgggggacctcggacgtga

SEQ ID NO: 23 - protein, 782 aa, Mus musculus FGFR3-delAcid
MVVPACVLVFCVAVVAGATSEPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 24 - DNA, 2349 bp, Mus musculus FGFR3-delAcid
atggtagtcccggcctgcgtgctagtgttctgcgtggcggtcgtggctggagctacttccgagcctcctggtccagagcagcgagttgtgcggagagcggcagaggttccagggcctgaacctagccagcaggagcaggtggccttcggcagtggggacaccgtggagctgagctgccatcctcctggaggtgcccccacagggcccacggtctgggctaaggatggtacaggtctggtggcctcccaccgcatcctggtggggcctcagaggctgcaagtgctaaatgcctcccacgaagatgcaggggtctacagctgccagcaccggctcactcggcgtgtgctgtgccacttcagtgtgcgtgtaacaggggctccttattggactcgcccggagcgaatggataagaaactgctggctgtgccagccgcaaacactgtccgcttccgctgcccagctgctggcaaccctaccccctccatctcctggctgaagaatggcaaagaattccgaggggagcatcgcattgggggcatcaagctccggcaccagcagtggagcttggtcatggaaagtgtggtaccctccgatcgtggcaactatacctgtgtagttgagaacaagtttggcagcatccggcagacatacacactggatgtgctggagcgctccccacaccggcccatcctgcaggctgggctgccggccaaccagacagccattctaggcagtgacgtggagttccactgcaaggtgtacagcgatgcacagccacacatccagtggctgaagcacgtggaagtgaacggcagcaaggtgggccctgacggcacgccctacgtcactgtactcaagactgcaggcgctaacaccaccgacaaggagctagaggttctgtccttgcacaatgtcacctttgaggacgcgggggagtacacctgcctggcgggcaattctattgggttttcccatcactctgcgtggctggtggtgctgccagctgaggaggagctgatggaaactgatgaggctggcagcgtgtacgcaggcgtcctcagctacggggtggtcttcttcctcttcatcctggtggtggcagctgtgatactctgccgcctgcgcagtcccccaaagaagggcttgggctcgcccaccgtgcacaaggtctctcgcttcccgcttaagcgacaggtgtccttggaatctaactcctctatgaactccaacacaccccttgtccggattgcccggctgtcctcaggagaaggtcctgttctggccaatgtttctgaacttgagctgcctgctgaccccaagtgggagctatccaggacccggctgacacttggtaagcctcttggagaaggctgctttggacaggtggtcatggcagaagctattggcatcgacaaggaccgtactgccaagcctgtcaccgtggccgtgaagatgctgaaagatgatgcgactgacaaggacctgtcggacctggtatctgagatggagatgatgaaaatgattggcaagcacaagaacatcattaacctgctgggggcgtgcacacagggtgggcccctgtatgtgctggtggagtacgcagccaagggcaatctccgggagttccttcgggcgcggcggcctccaggcatggactactcctttgatgcctgcaggctgccagaggaacagctcacctgcaaggatctagtgtcctgtgcctaccaggtggcacggggcatggaatacttggcttctcagaagtgtattcacagagacttggctgccagaaacgtcctggtgaccgaggacaatgtgatgaagattgcggactttggcctggctcgagatgtgcacaacctggactactacaagaagaccacaaatggccggctacctgtgaagtggatggcaccagaggccctttttgaccgagtctacacccaccagagtgatgtttggtcttttggtgtcctcctctgggagatctttacgctggggggctcaccgtatcctggcatcccagtggaagagcttttcaagctgttgaaagagggccaccgcatggacaagccagccagctgcacacatgacctgtacatgatcatgcgggaatgttggcatgcggtgccttcacagaggcccaccttcaagcagttggtagaggatttagaccgcatcctcactgtgacatcaaccgacgagtacttggacctctccgtgccgtttgagcagtactcgccaggtggccaggacacgcctagctccagctcgtccggagatgactcggtgttcacccatgacctgctacccccaggtccacccagtaacgggggacctcggacgtga

SEQ ID NO: 25 - protein, 802 aa, Rattus norvegicus FGFR3IIIb
MVVPACVLVFCVAVVAGVTSEPPGPEQRVGRRAAEVPGPEPSQQEQVAFGSGDTVELSCHPPGGAPTGPTLWAKDGVGLVASHRILVGPQRLQVLNATHEDAGVYSCQQRLTRRVLCHFSVRVTDAPSSGDDEDGEDVAEDTGAPYWTRPERMDKKLLAVPAANTVRFRCPAAGNPTPSIPWLKNGKEFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISENVEADARLRLANVSERDGGEYLCRATNFIGVAEKAFWLRVHGPQAAEEELMEVDEAGSVYAGVLSYGVGFFLFILVVAAVTLCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 26 - DNA, 2409 bp, Rattus norvegicus FGFR3IIIb
atggtagtcccggcctgcgtgctagtgttctgcgtggcggtcgtggctggagttacttccgagcctcccggtccagagcagcgagttggtcggagagcggcagaggttccagggcctgaacctagccagcaggagcaggtggccttcggcagtggggacactgtggagctgagctgccatccgcctggaggtgcccccacaggccccactctctgggctaaggacggtgtggggctggtggcctcccaccgtatcctggtggggcctcagaggcttcaagtgctaaacgccacccatgaggatgctggggtctacagctgccagcagcggctaacccggcgtgtgctgtgccactttagtgtgcgtgtaacagatgctccgtcctcaggagatgacgaagatggggaggacgtggctgaagacacaggggctccttactggactcgaccggagcgtatggataagaaactgctggctgtgccagctgcaaacactgtacgcttccgctgcccagctgctggcaaccccaccccctccatcccctggctgaagaacggcaaagaattccgaggggagcaccgcattgggggcattaagctccggcaccagcagtggagcttggtcatggaaagtgtggtgccctctgaccgcggcaattacacctgcgtggttgagaacaagtttggcagcatccggcagacgtacaccctggatgtgctggagcgctccccacaccggcccatcctgcaggctgggctgccagccaaccagacagccgttctgggcagtgacgtggagttccactgcaaggtgtacagcgacgcacagccacacatccagtggctgaagcacgtggaggtgaatgggagcaaggtgggccctgacggcacgccctacgtcactgtactcaagtcctggatcagtgagaatgtggaggcagacgcacgcctccgcctggccaatgtgtcggagcgggacgggggcgagtacctctgtcgagccaccaatttcataggcgtggccgagaaggccttttggcttcgtgttcacgggccccaagcagccgaggaggagctgatggaagttgacgaggctggcagcgtgtacgcgggtgtcctcagctacggggtgggcttcttcctcttcatcctggtggtggcggcagtgacgctctgccgtctgcgcagtcccccaaagaagggcctgggctcgcccaccgtgcacaaggtctctcgcttcccgcttaagcgacaggtgtccttggagtctaattcctctatgaactccaacacacctctcgtccggattgcccggctgtcctcaggagaaggtcctgtcctggccaatgtttctgaacttgagctgcctgctgaccccaagtgggagctatccaggacccggctgacactcggtaagcctcttggagaaggctgctttggacaggttgtcatggcagaagctattggcatcgacaaggaccgcactgccaagcctgtcaccgtggccgtgaagatgctgaaagatgatgcgactgacaaggacctgtcggacctggtgtctgagatggagatgatgaaaatgattggcaagcacaagaacatcattaacctgttgggggcctgcacccagggtgggcccctgtatgtgctggtggagtatgcagccaagggcaacctgcgagagttcctccgggcacggcggcctccaggcatggattactcctttgatgcctgcaggctgccagaggaacagctcacctgcaaggatctggtgtcctgtgcctaccaggtggcacggggcatggagtacttggcttcccagaagtgtattcacagagacctggctgccagaaacgtgctggtgactgaggacaatgtgatgaagattgcagactttggcctggcccgagatgtgcacaacctggattactacaagaagaccacaaatggccggctacctgtgaagtggatggcaccagaggccctttttgaccgagtctacacccatcagagtgatgtctggtcctttggtgtcctcctctgggagatctttacactgggtgggtcaccatatcctggcatcccagtggaagagcttttcaagctgttgaaagagggccaccgcatggacaagccagccaactgcacacatgacctgtacatgatcatgcgggaatgttggcatgcagtgccttcacagaggcccaccttcaagcagttggtagaggatttagaccgcatcctcacggtgacatcaactgacgagtacttggacctctcggtgccatttgaacagtactcgccaggtggccaagatactcctagctccagctcgtccggggacgactctgtgttcacccatgacctgctacccccaggcccacccagcaatgggggacctcggacgtga

SEQ ID NO: 27 - protein, 800 aa, Rattus norvegicus FGFR3IIIc
MVVPACVLVFCVAVVAGVTSEPPGPEQRVGRRAAEVPGPEPSQQEQVAFGSGDTVELSCHPPGGAPTGPTLWAKDGVGLVASHRILVGPQRLQVLNATHEDAGVYSCQQRLTRRVLCHFSVRVTDAPSSGDDEDGEDVAEDTGAPYWTRPERMDKKLLAVPAANTVRFRCPAAGNPTPSIPWLKNGKEFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDRELEVLSLHNVTFEDAGEYTCLAGNSIGFSHHSAWLVVLPAEEELMEVDEAGSVYAGVLSYGVGFFLFILVVAAVTLCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 28 - DNA, 2403 bp, Rattus norvegicus FGFR3IIIc
atggtagtcccggcctgcgtgctagtgttctgcgtggcggtcgtggctggagttacttccgagcctcccggtccagagcagcgagttggtcggagagcggcagaggttccagggcctgaacctagccagcaggagcaggtggccttcggcagtggggacactgtggagctgagctgccatccgcctggaggtgcccccacaggccccactctctgggctaaggacggtgtggggctggtggcctcccaccgtatcctggtggggcctcagaggcttcaagtgctaaacgccacccatgaggatgctggggtctacagctgccagcagcggctaacccggcgtgtgctgtgccactttagtgtgcgtgtaacagatgctccgtcctcaggagatgacgaagatggggaggacgtggctgaagacacaggggctccttactggactcgaccggagcgtatggataagaaactgctggctgtgccagctgcaaacactgtacgcttccgctgcccagctgctggcaaccccaccccctccatcccctggctgaagaacggcaaagaattccgaggggagcaccgcattgggggcattaagctccggcaccagcagtggagcttggtcatggaaagtgtggtgccctctgaccgcggcaattacacctgcgtggttgagaacaagtttggcagcatccggcagacgtacaccctggatgtgctggagcgctccccacaccggcccatcctgcaggctgggctgccagccaaccagacagccgttctgggcagtgacgtggagttccactgcaaggtgtacagcgacgcacagccacacatccagtggctgaagcacgtggaggtgaatgggagcaaggtgggccctgacggcacgccctacgtcactgtactcaagactgcaggagctaacaccaccgacagggagctagaggttctgtccttgcacaatgtcacctttgaggatgcgggggagtacacctgcctggcgggcaattctatcgggttttcccatcactctgcgtggctggtggtgctgccagccgaggaggagctgatggaagttgacgaggctggcagcgtgtacgcgggtgtcctcagctacggggtgggcttcttcctcttcatcctggtggtggcggcagtgacgctctgccgtctgcgcagtcccccaaagaagggcctgggctcgcccaccgtgcacaaggtctctcgcttcccgcttaagcgacaggtgtccttggagtctaattcctctatgaactccaacacacctctcgtccggattgcccggctgtcctcaggagaaggtcctgtcctggccaatgtttctgaacttgagctgcctgctgaccccaagtgggagctatccaggacccggctgacactcggtaagcctcttggagaaggctgctttggacaggttgtcatggcagaagctattggcatcgacaaggaccgcactgccaagcctgtcaccgtggccgtgaagatgctgaaagatgatgcgactgacaaggacctgtcggacctggtgtctgagatggagatgatgaaaatgattggcaagcacaagaacatcattaacctgttgggggcctgcacccagggtgggcccctgtatgtgctggtggagtatgcagccaagggcaacctgcgagagttcctccgggcacggcggcctccaggcatggattactcctttgatgcctgcaggctgccagaggaacagctcacctgcaaggatctggtgtcctgtgcctaccaggtggcacggggcatggagtacttggcttcccagaagtgtattcacagagacctggctgccagaaacgtgctggtgactgaggacaatgtgatgaagattgcagactttggcctggcccgagatgtgcacaacctggattactacaagaagaccacaaatggccggctacctgtgaagtggatggcaccagaggccctttttgaccgagtctacacccatcagagtgatgtctggtcctttggtgtcctcctctgggagatctttacactgggtgggtcaccatatcctggcatcccagtggaagagcttttcaagctgttgaaagagggccaccgcatggacaagccagccaactgcacacatgacctgtacatgatcatgcgggaatgttggcatgcagtgccttcacagaggcccaccttcaagcagttggtagaggatttagaccgcatcctcacggtgacatcaactgacgagtacttggacctctcggtgccatttgaacagtactcgccaggtggccaagatactcctagctccagctcgtccggggacgactctgtgttcacccatgacctgctacccccaggcccacccagcaatgggggacctcggacgtga

SEQ ID NO: 29 - protein, 806 aa, Gallus gallus FGFR3
MRAAWGSVWCLCLAAAVGALPAARRRGAERSGGQAAEYLRSETAFLEELVFGSGDTIELSCNTQSSSVSVFWFKDGIGIAPSNRTHIGQKLLKIINVSYDDSGLYSCKPRHSNEVLGNFTVRVTDSPSSGDDEDDDDESEDTGVPFWTRPDKMEKKLLAVPAANTVRFRCPAGGNPTPTIYWLKNGKEFKGEHRIGGIKLRHQQWSLVMESVVPSDRGNYTCVVENKYGNIRHTYQLDVLERSPHRPILQAGLPANQTVVVGSNVEFHCKVYSDAQPHIQWLKHVEVNGSKYGPDGTPYVTVLKTAGVNTTDKELEILYLRNVTFEDAGEYTCLAGNSIGFSHHSAWLTVLPAEELMEMDDSGSVYAGILSYGTGLVLFILVLVIVIICRMKMPNKKAMNTTTVQKVSKFPLKRQQVSLESNSSMNSNTPLVRITRLSSSDGPMLANVSELELPPDPKWELARSRLTLGKPLGEGCFGQVVMAEAIGIDKDKPNKAITVAVKMLKDDATDKDLSDLVSEMEMMKMIGKHKNIINLLGACTQDGPLYVLVEYASKGNLREYLRARRPPGMDYSFDTCKLPEEQLTFKDLVSCAYQVARGMEYLASQKCIHRDLAARNVLVTEDNVMKIADFGLARDVHNIDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRVLTMTSTDEYLDLSVPFEQYSPAGQDTHSTCSSGDDSVFAHDLLPDEPCLPKHVPCNGVIRT

SEQ ID NO: 30 - DNA, 2421 bp, Gallus gallus FGFR3
atgcgggcggcctggggctccgtctggtgcctgtgcctggcggcggccgtcggagcgctgccggcggcgcgccggcgcggagcggagcggagcggcgggcaggcggcagaatacttgaggagcgagaccgcctttctggaagagttggtgtttggaagtggagataccattgaactttcctgtaacacccagagctcttctgtgtcagttttctggtttaaagatggtattgggattgcaccttccaacagaactcatattggacaaaaactgttgaagataatcaatgtgtcatatgacgattcggggctgtacagttgcaagccaaggcattccaacgaggtcctgggaaactttacagtcagagtgacagattccccttcgtcaggtgatgatgaagatgatgacgatgagtcagaggatacaggtgtccccttctggacccggccagataagatggagaagaagctgctggcagttcctgccgccaacaccgttcgcttccgatgtccagcaggtggaaacccaactcccaccatttactggctgaagaatggcaaagaattcaagggagagcacaggatcgggggcatcaagttgcgacaccagcagtggagcttggtgatggagagcgttgtgccgtcagatcgaggaaactacacctgtgttgtggagaacaaatatggcaatattaggcacacataccagcttgatgttttagaacggtcaccccaccgaccaatcctgcaagcaggactccctgccaatcagactgtggtggtcgggagcaatgtggaatttcactgcaaggtctacagcgatgcccagcctcatatccagtggctgaaacacgtagaagtcaacggcagcaagtatggacctgatgggacaccctatgtcacagtgctgaagacggcaggtgttaacacaacggataaggagctagagattctgtacttgcgaaatgttacttttgaggatgctggggaatatacttgtctcgcagggaattctattgggttctcacatcactctgcttggctgacggtgctaccagcagaggagctgatggaaatggatgattcgggctcagtgtacgctggcattctcagctatggcactggcttagtcctcttcatcctggtgctggtcattgtgattatctgcaggatgaaaatgccaaacaaaaaggccatgaacaccaccactgtacagaaagtctccaaatttccactcaagagacagcaggtgtcgttggagtccaactcttccatgaattccaacacacccctggtccggatcactcgtctctcctccagcgatgggccgatgctggccaacgtctctgagctggaacttcctccagatcccaagtgggaattggcacgttctcgcctgaccctggggaagccgcttggtgagggctgttttggccaagtggtgatggcggaagcaattgggattgataaagacaagccaaacaaggccatcaccgtggctgtcaagatgttaaaagatgatgccacagacaaggacctttcagacctggtctctgagatggaaatgatgaaaatgattgggaagcacaaaaacatcattaacctgctcggtgcttgcacgcaggacggaccgctctacgtgttggttgaatatgcatcgaaggggaacttgcgggaatacctcagggcacgtcgcccacctggcatggactattccttcgacacctgcaagctgcccgaggagcagttgacatttaaagacctggtttcctgcgcctaccaggtggcccggggcatggagtacttggcgtcacagaaatgcattcatcgtgacttggcagccaggaatgtgttagtcactgaggacaatgtgatgaaaatagctgattttggccttgctagagacgttcacaacatcgactattacaagaaaaccaccaatggtcggctgcctgtgaaatggatggctccagaagcattgtttgaccgggtctatactcaccagagcgatgtctggtcttttggagtgctactatgggagatcttcactttgggagggtctccgtacccgggaattcctgttgaagaactcttcaaactcttgaaagaaggccatcggatggataaacccgccaactgtacccacgacctgtacatgatcatgcgggagtgctggcacgctgtcccctcgcagcgacccacattcaagcagctggtggaagacctggacagagtcctcaccatgacatccactgatgagtacctggacctctcggtgccctttgagcaatactcacccgctggccaggacacccacagcacctgctcctcaggggacgactcggtttttgcacatgacctgctgcctgatgagccctgcctgcccaagcacgtgccctgtaatggcgtcatccgcacgtga

SEQ ID NO: 31 - protein, 827 aa, Xenopus laevis FGFR3-1
MSKAGGGCGIALYQGIHMGIVTLFCTLCFFLVSVNCVPAARLPVTLPGEDRANRKASDYLTVEQPPFDELMFTIGETIELSCSADDASTTTKWFKDGIGIVPNNRTSTRQGLLKIINISYDDSGIYSCRLWHSTEILRNFTIRVTDLPSSGDDEDDDDETEDREPPRWTQPEKMEKKLIAVPAANTIRFRCPAAGNPTPTIHWLKNGKEFRGEHRIGGIKLRHQQWSLVMESVVPSDKGNYTCVVENKYGSIRQTYQLDVLERSSHRPILQAGLPANQTVVFGSDVEFHCKVYSDAQPHIQWLKHVEVNGSKYGPDGDPYVTVLQSFTNGTEVDSTLSLKNVTETHEGQYVCRANNFIGVAEASFWLHIYKPAPAEPVEKPATTSSSSITVLIVVTSTIVFILLVIIVITYRMKVPSKKAMSTPPVHKVSKFPLKRQVSLESNSSMNSNTPLVRITHLSSSDGTMLANVSELGLPLDPKWELLRSRLTLGKPLGEGCFGQVVMAEAIGIDKERPNKPVTVAVKMLKDDATDKDLSDLVSEMEMMKMIGKHKNIINLLGACTQDGPLYVLVEYASKGNLREYLKARRPPGMDYSFDTCKIPAEQLTFKDLVSCAYQVARGMEYLASQKCIHRDLAARNVLVTDDIVMKIADFGLARDIHNIDYYKKTTNGRLPVKWMAPEALFDRIYTHQSDVWSYGVLLWEIFTLGGSPYPGIPVEELFKLLKEGHRMDKPANCTHELYMIMRECWHAVPSQRPTFKQLVEDLDRVLTVTSTDEYLDLSVPFEQYSPAGQDSNSTCSSGDDSVFAHDILPDEPCLPKQQQYNGAIRT

SEQ ID NO: 32 - DNA, 2484 bp, Xenopus laevis FGFR3-1
atgtctaaggctggagggggctgtggaattgccctttatcaagggatccatatgggaattgtcaccctgttctgcactctctgcttttttctggtctctgtgaactgtgtcccggctgcccgactgccagttacgctccctggagaggacagagcaaacagaaaagcatcagattatctcacggtagaacagcccccattcgatgagctcatgtttacaattggagaaaccattgagttgtcctgctctgcggatgatgcatccacgaccaccaagtggttcaaggatggtatcggcattgtgccgaacaacagaacaagtacgaggcagggcctgctgaagattatcaacatctcatacgatgactctgggatatacagttgcagactatggcattctactgaaattctgcgcaattttaccatcagagtaacagacttaccatcgtccggtgatgatgaggatgacgatgatgaaaccgaagacagagagcctcctcgctggacccaacctgagaagatggagaagaaacttattgcagtccctgccgctaacacaatccgattccggtgcccagccgcggggaatcccacccctaccatccattggcttaagaacggaaaggaattcaggggagagcatcgtattggtggcatcaaactccgacatcagcagtggagcctcgttatggagagcgtagttccatcggataaaggcaactacacgtgtgtagtggagaacaaatatggaagcatccgtcaaacctatcaacttgatgtcctggagaggtcctctcaccggcccatccttcaggccgggttacccgccaaccagacggtggtgtttgggagcgacgtggaattccactgcaaagtctacagtgacgcacagccacatattcagtggcttaaacacgtggaagtgaatggcagcaagtacggcccagacggagatccttacgtcacagtgctgcaatctttcaccaatggcactgaagtcgattctaccttaagtctaaaaaatgtgaccgagacccatgaaggacagtatgtgtgtagagccaacaatttcataggagtagccgaggcatccttttggctccacatttacaaaccagcaccagcagaaccagtggagaagccagcaaccacatcttccagctccatcaccgttcttattgtggtcacctcgactattgtgttcatactgttggttatcattgtcatcacctaccgcatgaaggtcccttctaagaaggcaatgagcaccccgccggtgcataaagtctccaagttcccgctcaagcggcaggtgtctctagagtccaactcttctatgaattccaacaccccgctggtgaggatcactcacctgtcctccagcgacggaaccatgttggctaatgtgtcggagctcggcctgcccctggatcccaagtgggagttattgagatcaaggctgactttaggaaagccccttggagaaggctgctttggtcaagtagtgatggcagaagcaattggcattgataaggaaaggccaaataagcctgttactgtagctgtaaagatgcttaaagatgatgctacagataaagatctctccgatctggtctcggagatggagatgatgaaaatgattgggaagcacaaaaatatcatcaatctgctaggagcatgcactcaggatggaccactgtacgttcttgtggaatatgcatccaaagggaacctcagggagtatttaaaggcacggcgccccccaggaatggattattcttttgacacctgcaaaattccagctgagcagctgacgttcaaggacctcgtttcttgcgcctaccaggtagctcgtggcatggagtacctggcgtcgcaaaaatgtattcacagagatctggcagccagaaatgtgttagtaacagatgacattgtaatgaagattgcagatttcggcttggccagggacatccacaacatagattattacaagaaaacaacaaatggtcggctgccagtcaaatggatggctccggaagctttgttcgaccgtatctacactcatcagagcgatgtatggtcgtacggagtgctgctgtgggagatatttacactggggggctcgccctacccagggatcccagtagaggaactctttaagctattgaaagaaggccacagaatggacaagccagcaaactgcacacatgaactgtatatgatcatgagagagtgctggcacgctgtcccatcgcaaagaccaaccttcaagcagctggttgaagaccttgaccgcgttcttactgtaacatctactgatgagtacctggacctgtcggtaccattcgagcagtattccccggcgggccaagacagtaacagcacctgctcctcgggggacgactcagtctttgctcatgacattttacccgatgaaccgtgtcttcccaaacaacagcagtacaacggcgccatccgaacatga

SEQ ID NO: 33 - protein, 802 aa, Xenopus laevis FGFR3-2
MVSVNGVPAARLPVTLPGEDRASRKAPDYLMVEQPPFDELMYTIGETIELSCAAEDASTTTKWCKDGIGIVPNNRTSTRQGLLKIINVSSDDSGIYSCRLWHSTEILRNFTIRVTDLPSSGDDEDDDDDDDDETEDREPPRWTQPERMEKKLIAVPAANTIRFRCPAAGNPTPTIHWLKNGKEFRGEHRIGGIKLRHQQWSLVMESVVPSDKGNYTCVVENKYGSIRQTYQLDVLERSSHRPILQAGLPGNQTVVLGSDVEFHCKVYSDAQPHIQWLKHVEVNGSKYGPDGDPYVSVLQSFINGTEVDSTLSLKNVTETNEGQYVCRANNFIGVAEASFWLHIYKPAPAEPVEKALTTSSSSITVLIVVTSTIVFILLVIIVITHLMKVPSKKSMTAPPVHKVSKFPLKRQQVSLESNSSMNSNTPLVRITHLSSSDGTMLANVSELGLPLDPKWELLRSRLTLGKPLGEGCFGQVVMAEAIGIDKERPNKPATVAVKMLKDDATDKDLSDLVSEMEMMKMIGKHKNIINLLGACTQDGPLYVLVEYASKGSLREYLKARRPPGMDYSFDACKIPAEQLTFKDLVSCAYQVARGMEYLASQKCIHRDLAARNVLVTDDNVMKIADFGLARDIHNIDYYKKTTNGRLPVKWMAPEALFDRIYTHHSDVWSYGVLLWEIFTLGGSPYPGIPVEELFKLLKEGHRMDKPANCTHELYMIMRECWHAVPSQRPAFKQLVEDLDRVLTVTSTNEYLDLSVAFEQYSPPSQDSHSTCSSGDDSVFAHDILPDEPCLPKHQQHNGAIPT

SEQ ID NO: 34 - DNA, 2409 bp, Xenopus laevis FGFR3-2
atggtctctgtgaatggtgtcccggctgcccgactgccagttacgctccctggagaggacagagcgagcagaaaagcaccagattatctcatggtagaacagcccccattcgatgaactcatgtatacaattggagaaaccattgagttgtcctgcgctgcagaagatgcttccacaactaccaagtggtgtaaggatggtattggcattgtaccgaacaacagaacaagcacaaggcagggcctgctgaagattatcaacgtctcctccgatgactccgggatatacagctgcagactatggcattctaccgagattctgcgcaattttacaatcagagtaacagacttgccatcatctggtgacgatgaggatgatgatgatgatgatgatgatgaaaccgaagacagagaacctcctcgctggacccaacctgagaggatggaaaagaaacttattgcagtccctgctgctaacacaatccgcttccggtgcccagccgcagggaatcccacccctaccatccactggctaaagaacggaaaggagttcaggggggaacatcgtattggtggcatcaaactccgacatcaacagtggagccttgttatggagagtgtggtcccatcagataaaggcaactacacgtgtgtggtggagaacaaatatggaagcatccgtcaaacctatcaacttgatgtccttgagaggtcctctcaccggcccatccttcaggctgggttacccggcaaccagacggttgtgcttgggagcgacgtggaattccactgcaaagtctacagtgacgcacaacctcatattcagtggcttaaacacgtggaagtgaatggcagcaaatacggcccagacggagatccttacgtctcagtgttgcaatctttcatcaatggcactgaagtcgattctaccctaagtctaaaaaatgtgaccgagaccaatgaaggacagtatgtgtgtagagccaacaatttcataggagtagccgaggcatccttttggctccacatttacaaaccagcaccagcagaaccagtggagaaggcattgacaacatcttccagctctatcaccgtccttattgtggtcacctcgaccattgtgttcatactgttggttatcatcgtcatcacccacctcatgaaggtcccttccaagaagtcaatgaccgccccaccggtgcataaagtctccaagttccccctcaaacggcagcaggtgtctctagagtccaactcttctatgaattccaacaccccgttggtgaggatcactcatctgtcctccagcgatggaaccatgctggctaatgtgtcggaacttggcctgccacttgaccccaagtgggagttattgagatcaaggctgactttaggaaagcccctcggggaaggctgcttcggtcaggtggtgatggcagaagctattggcattgataaggaaaggccaaataagcctgctactgtagctgtaaagatgcttaaagacgatgccacagataaagatctctcagatctggtctctgagatggagatgatgaaaatgattgggaagcataaaaatatcatcaatctgctgggagcatgcactcaggatgggccgctgtacgttctggtggaatacgcatcgaaagggagcctcagggagtatttaaaggcacggcgccccccaggaatggattattcttttgatgcctgcaaaattccagctgagcagctgacgttcaaggacctagtttcttgtgcctaccaggtagctcgtggcatggagtacctggcatcacaaaaatgcattcacagagatctggcagccagaaatgtgttagtaacagatgacaacgtaatgaagattgcagatttcggcttggccagggacatccacaacatagattattacaagaaaacaacaaatggtcggctgcctgtgaaatggatggctccggaagctttgtttgaccgtatctacactcatcacagcgatgtatggtcgtacggagtgctgctgtgggagatatttacactggggggctcaccctacccagggatcccggtagaggaactttttaagctattgaaagaaggccacagaatggacaagccagcaaactgcacacatgaactgtatatgatcatgagagagtgctggcacgctgtcccctcacaaagacccgccttcaagcagctggttgaagaccttgaccgcgttcttactgtaacatctactaatgagtacctagacctctcggtagcattcgagcagtattctccacccagccaagacagtcacagcacctgctcctcaggggacgactcagtctttgctcacgacattttacccgatgaaccgtgtcttcccaaacaccagcagcacaacggcgccatccccacatga

SEQ ID NO: 35 - protein, 796 aa, Pleurodeles waltlii FGFR3
MLVWLCGLCLVTLAGGRSAARLPLTEGRPTADFLPGDASLVEELLFGTGDTIELSCTTPGSSVSVVWFKDGISVDPPTWSHTGQKLLKIINVSYDDSGVYSCKARQSSEVLRNVTVRVTDSPSSGDDEDDDEESESANAPKFTRPEWMEKKLLAVPAANTVRFRCPAAGKPTPSITWLKNGKEFKGEHRIGGIKLRHQQWSLVMESVVPSDRGNYTCVVANKYGTIRETYTLDVLERTPHRPILQAGFRSNKTVVVGSDVEFHCKVYSDAQPHIQWLKHVEVNGSKFGPDGNPYVTVLKTAGVNTSDKELEIQFLRNVTFEDAGEYTCLAGNSIGYSHHSAWLTVLPPAEPVPDVDTSVSILAAAGCVAVVILVVIIIFTYKMKMPSKKTMNTATVHKVSKFPLKRQVSLESNSSMNSNTPLVRITRLSSSDGPMLANVSELELPADPKWELSRSRLTLGKPLGEGCFGQVVMADAVGIEKDKPNKATSVAVKMLKDDATDKDLSDLVSEMEMMKMIGKHKNIINLLGACTQDGPLYVLVEYASKGNLREYLRARRPPGMDYSFDTCKLPEEQLTFKDLVSCAYQVARGMEYLASQKCIHRDLAARNVLVTDDNVMKIADFGLARDVHNIDYYKKTTNGRLPVKWMAPEALFDRVYTHQSDVWSFGVLLWEIFTLGGSPYPGIPVEELFKLLKEGHRMDKPANCTHELYMIMRECWHAVPSQRPTFKQLVEDLDRVLTVTSTDEYLDLSVPFEQYSPACPDSHSSCSSGDDSVFAHDLPEEPCLPKHQQYNGVIRT

SEQ ID NO: 36 - DNA, 2391 bp, Pleurodeles waltlii FGFR3
atgctcgtctggctctgcggcttgtgtctggtgactctggcgggcggacgttcggcggccaggctgcccctcaccgagggccgacccacagcagacttcctgcccggcgacgcctccctggtggaagagctcctgttcggcacgggggacaccatcgagctctcctgcaccaccccgggctcctctgtgtccgtggtgtggttcaaagacgggatctcggtggacccaccaacctggtcccacaccggccagaagctgctgaagatcatcaacgtgtcctacgacgactcgggagtgtacagctgcaaggcccggcagtccagcgaggtgctccggaacgtgaccgtcagggtgaccgattctccgtcatccggtgatgacgaagatgatgatgaggaatctgaaagtgcaaatgcaccaaaattcacgcgaccggaatggatggagaagaaactgcttgcagtgcccgcagccaacacggtgcgcttccgatgcccagctgcaggaaagccaacgccatccatcacttggctgaaaaacggcaaggagttcaaaggcgagcatcggattgggggcataaagctaagacaccagcagtggagtttggtgatggagagtgtagtcccatccgatcggggaaattacacatgtgtggtggcaaacaagtacggcaccatccgagagacctacacattggatgtccttgaacgaactcctcaccggcccatcctccaggcgggattccgttccaacaagactgtggtggtaggaagcgatgtggagttccattgcaaggtatacagtgatgctcagccgcacatccagtggctgaaacacgtggaggttaatggcagcaagtttggacctgatgggaacccgtatgtcacagtgcttaagacggcaggtgttaatacctcggataaggagctagaaattcagttcttgcgaaatgtaacttttgaggatgctggggagtatacttgtctcgctgggaactctattggctattcccatcattctgcttggctcacggtgctgccaccagcagagccggtcccagacgtcgacacctctgtcagcattcttgccgctgcaggatgtgtcgcagttgttatactggtggtgatcataatctttacttacaagatgaagatgccctccaagaagaccatgaacaccgccactgtgcacaaagtctcaaagttccctctcaagagacaggtgtcactggagtccaactcttcaatgaattccaacacccctctggtgcgaatcacccgcctgtcgtccagcgatggtccgatgctggccaacgtgtccgagctggagctacccgctgatccgaagtgggaattgtctcgttcacgcttgactttgggcaaacctcttggggaaggatgctttggccaggtggtgatggcggatgcagttggcattgaaaaggataagccaaacaaggccacctcggttgccgttaagatgttgaaagatgatgccactgataaagacctgtcggatctagtctctgaaatggaaatgatgaaaatgattgggaagcacaaaaacatcattaatctcctgggagcctgcacgcaggatggcccactctacgtgctggtggaatatgcatccaaaggaaacttgcgggagtacctgagggcccggcgccctcctggcatggattactccttcgacacctgcaaacttcccgaagagcagttgaccttcaaggacttggtatcctgtgcctaccaggtggcccgcggcatggagtacctggcctctcagaagtgcatacaccgagatctggcagcccggaacgtgctggtgacggatgacaacgttatgaagattgctgattttggcctggcgagagatgtgcacaacatcgactactacaagaaaactacaaatggccgactgcccgtgaagtggatggctccggaggctttgttcgaccgggtctacactcaccaaagcgacgtctggtcgtttggagtgcttctgtgggagatcttcacgctggggggctcgccgtaccctggaatcccagtggaagaactcttcaagctgttaaaggaaggccatcgaatggacaaaccagcgaactgcacgcatgagctgtacatgatcatgcgggagtgctggcatgcagtgccatcccagcggccaaccttcaagcaactcgtagaagacttggaccgggtccttacggtgacctccactgatgagtacctcgatctctctgtgcccttcgagcagtattcgcctgcctgcccagacagccacagcagctgctcttctggagacgattcggtctttgcccacgacctgcccgaggagccctgccttccgaagcaccagcagtacaatggagtaatccgaacatga

SEQ ID NO: 37 - protein, 800 aa, Danio rerio FGFR3
MVPLCLLLYLATLVFPPVYSAHLLSPEPTDWVSSEVEVFLEDYVAGVGDTVVLSCTPQDFLLPIVWQKDGDAVSSSNRTRVGQKALRIINVSYEDSGVYSCRHAHKSMLLSNYTVKVIDSLSSGDDEDYDEDEDEAGNGNAEAPYWTRSDRMEKKLLAVPAANTVKFRCPAAGNPTPSIHWLKNGKEFKGEQRMGGIKLRHQQWSLVMESAVPSDRGNYTCVVQNKYGSIKHTYQLDVLERSPHRPILQAGLPANQTVVVGSDVEFHCKVYSDAQPHIQWLKHIEVNGSQYGPNGAPYVNVLKTAGINTTDKELEILYLTNVSFEDAGQYTCLAGNSIGYNHHSAWLTVLPAVEMEREDDYADILIYVTSCVLFILTMVIIILCRMWINTQKTLPAPPVQKLSKFPLKRQVSLESNSSMNSNTPLVRIARLSSSDGPMLPNVSELELPSDPKWEFTRTKLTLGKPLGEGCFGQVVMAEAIGIDKEKPNKPLTVAVKMLKDDGTDKDLSDLVSEMEMMKMIGKHKNIINLLGACTQDGPLYVLVEYASKGNLREYLRARRPPGMDYSFDTCKIPNETLTFKDLVSCAYQVARGMEYLASKKCIHRDPAARNVLVTEDNVMKIADFGLARDVHNIDYYKKTTNGRLPVKWMAPEALFDRVYTHQSDVWSYGVLLWEIFTLGGSPYPGIPVEELFKLLKEGHRMDKPANCTHELYMIMRECWHAVPSQRPTFRQLVEDHDRVLSMTSTDEYLDLSVPFEQYSPTCPDSNSTCSSGDDSVFAHDPLPEEPCLPKHHHSNGVIRT

SEQ ID NO: 38 - DNA, 2403 bp, Danio rerio FGFR3
atggtcccactctgtctcctcctgtacctcgcaaccctcgtcttcccaccagtgtacagtgcacacctgctgtccccagagcccacagactgggtatcgagtgaggtggaagtgtttctggaggactatgtggcgggagtcggggatacagtagttctgtcctgcacgccgcaagactttctccttcccatcgtatggcaaaaagacggagacgccgtttcttcaagcaaccgtacacgagtgggccagaaagccctccgcatcatcaatgtctcctatgaagactcgggtgtttactcctgcagacatgcccacaagagcatgcttctgagcaactacaccgtcaaagtcatcgattcgctgtcctctggtgatgatgaggactatgatgaagatgaggacgaggcaggtaatggaaatgcagaagctccatactggacccgttcggaccggatggagaagaaactattggctgttcctgctgccaatacagtcaagttccgctgtcctgctgctggcaacccaacgcccagtatccattggctgaaaaatggcaaggagttcaagggagagcagagaatgggcggcattaagctgaggcatcagcagtggagcttggtcatggagagtgccgttccatccgaccggggaaattacacatgtgtggtgcagaacaaatacgggtcaatcaagcacacttatcaactcgatgtgctggagcgctcccctcaccggcccatcttacaggcaggactgccagccaatcagacggtagtggtgggcagtgatgtggagttccactgtaaggtgtacagtgatgctcagccacacatccagtggctgaaacacattgaagtcaatggaagccaatatgggcccaatggcgccccctacgtcaatgttcttaagactgctgggataaatactacggataaagagctggagattctctacctgaccaatgtgtctttcgaggatgcggggcaatacacttgtctggcagggaactcgattggctataaccatcactctgcttggcttacagtcttaccagcggtggagatggagagagaggatgattatgcagacatcctcatctatgtgacaagctgcgtgctcttcattctcaccatggtcatcattattctctgccgaatgtggataaacacgcagaagactctcccggcaccacctgttcaaaaactgtccaaattccccctcaagagacaggtgtccttggaatccaactcttccatgaattcaaacaccccgctggtcaggatcgcccgcctgtcatccagcgatgggccgatgttgcctaacgtgtctgaacttgaactgccctctgaccccaagtgggagtttactcgaacaaagttaacgttggggaaaccgttgggagagggctgctttgggcaggtggtgatggctgaagccattgggattgacaaagaaaaacccaacaaacctctaactgttgctgtcaagatgctcaaagatgacggcacagataaagacctgtcagaccttgtgtctgaaatggagatgatgaagatgattgggaaacataagaacatcattaacttgctgggagcatgtactcaagacggtcctctgtacgtgctggtagaatacgcctctaaagggaatcttagggaatacttacgagccagaaggccacctgggatggactactcattcgacacctgtaagatcccgaacgaaacgctaacatttaaagacctggtgtcctgcgcctatcaggtcgccaggggtatggagtacctggcctcaaagaagtgtatccatagggaccccgcagcccggaatgttctggttaccgaggacaacgtgatgaagattgcagacttcggccttgccagagatgtgcacaacattgactactacaagaagaccaccaacggtcgtctgcccgtcaaatggatggcaccagaagcactgttcgatcgcgtctacacgcaccagagcgatgtgtggtcttatggtgtgttgttgtgggagattttcactcttggtggatccccgtatccaggtatcccagtggaggagctctttaaactgctgaaggaaggccatcggatggacaaaccggccaactgcactcatgaactgtacatgatcatgcgagaatgttggcatgctgttccttcacaaagacccacgttcagacagctggtggaggaccacgacagggttctttccatgacctccactgacgagtacctggacctctctgtaccgttcgagcagtattcaccgacctgtccggactccaacagcacctgttcctctggcgatgactctgtgtttgcccacgaccccttacctgaggagccatgcctccctaaacaccaccacagcaacggggtcatacgaacataa

(19) United States
(12) Patent Application Publication
(10) Pub. No.: US 2008/0274480 A1
(43) Pub. Date: Nov. 6, 2008

(54) Botulinum Toxin Type a Immunoresistant Assay

(75) Inventor: M. Zouhair Atassi, Houston, TX (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/572,530

(22) PCT Filed: Aug. 2, 2005
(86) PCT No.: PCT/US2005/027849

§ 371 Date: Aug. 22, 2007

Related U.S. Application Data
(60) Provisional application No. 60/599,070, filed on Aug. 4, 2004.

(51) Int. Cl. G01N 33/569

(52) U.S. Cl. 435/7.21

(57) ABSTRACT

The present specification discloses methods for determining Botulinum Toxin Type A immunoresistance in a mammal by detecting the amount of BoNT/A toxin-BoNT/A receptor complexes or the amount of free BoNT/A present or absent in a sample.

This patent application claims priority pursuant to 35 U.S.C. §119(e) to provisional patent application Ser. No. 60/599,070 filed Aug. 4, 2004, which is hereby incorporated by reference in its entirety.

All of the patents and publications cited in this application are hereby incorporated by reference in their entirety. All GeneBank sequence listings cited this application, as identified by their GenBank accession numbers, are available from the National Center for Biotechnological Information and are all hereby incorporated by reference in their entirety.

The ability of Clostridial toxins, such as, e.g., Botulinum neurotoxins (BoNTs) BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). For example, BoNT/A has emerged as an important therapeutic treatment for a number of neurological and ophthalmic disorders that have few other effective remedies, such as, e.g., cervical dystonia (asymmetric muscular spasm in the neck that results in forceful turning of the head), strabismus (misalignment of the eyes), focal spasm, such as, e.g., hemifacial spasm (sudden unilateral muscle contractions of the face), and blepharospasm (forceful involuntary closure of the eyelids). One such BoNT/A preparation, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder. Subsequently, proposed uses of BoNT/A as a biopharmaceutical neuromodulator has expanded to cover a wide variety of disorders where chemodenervation of the neuromuscular junctions may be beneficial, such as, e.g., without limitation, chronic lower back pain, oromandibular dystonia (continuous spasms of the face, jaw, neck, tongue, larynx, and respiratory system), spasmodic dysphonia (spasm of the vocal cords that causes sudden disruption of speech), stuttering and voice tremors, and various focal and segmental dystonias. In addition, BoNT/A treatments targeting certain disorders that lack a neuromuscular basis were developed. For example, the effects on the autonomic nervous system that BoNT/A may be of use in treating axillary hyperhydrosis or sweating, and reports indicate BoNT/A may be an effective treatment for myofascial pain and tension, stroke, traumatic brain injury, cerebral palsy, gastrointestinal motility disorders, urinary incontinence cancer and migraine headaches. Lastly, cosmetic and other therapeutic applications are widely known. In fact, the expected use of BoNT/A in both therapeutic and cosmetic treatments of humans is anticipated to expand to an ever widening range of diseases and aliments that can benefit from the properties of this toxin.

The inhibition of neurotransmitter release and the resulting neuromuscular paralysis elicited by BoNTs and TeNT, is not permanent. The reversible nature of these paralytic effects requires periodic treatments in order to maintain the therapeutic benefits from this toxin. As a consequence of this repeated exposure, an immune response against a toxin can occur in some patients which reduce or completely prevent the individual's responsiveness to further treatments, see, e.g., Joseph Jankovic, Botulinum toxin: Clinical Implications of Antigenicity and Immunoresistance, (SCIENTIFIC AND THERAPEUTIC ASPECTS OF BOTULINUM TOXIN, 409-415, Mitchell F. Brin et al., eds., Lippincott Williams & Wilkins, 2002); Dirk Dressier, Clinical Presentation and Management of Antibody-induced Failure of Botulinum Toxin Therapy, 19(Suppl. 8) MOV. DISORD. S92-S100 (2004); M. Zouhair Atassi, Basic Immunological Aspects of Botulinum Toxin Therapy, 19(Suppl. 8) Mov. DISORD. S68-S84, (2004). The development of BoNT/A immunoresistance is associated with the appearance of neutralizing anti-BoNT/A antibodies found in their serum, see, e.g., Göschel et al., supra, (1997); Jankovic, supra, (2002); Atassi, supra, (2004). While an immune response against the toxin is not initially observed in an individual undergo BoNT/A therapy, continued treatment seems to cause the production of neutralizing BoNT/A antibodies in some patients. Thus, one of the primary means underlying this immune response against BoNT/A appears to be the production of neutralizing anti-BoNT/A antibodies that bind the toxin, thereby preventing its ability to properly function.

Assays that determine whether a patient is mounting an immune response against a BoNT or TeNT are of major importance. These assays allow the immunoresponsive state of the patient to be evaluated periodically during the course of a therapy. By knowing the predisposition of an individual 1) the potential value of a specific treatment can be determined prior to its administration to a patient; and 2) the possible benefit from continued therapy can be assessed and any possible adjustments to a treatment determined. Therefore, these assays present a major benefit in terms of providing better patient care and reducing health care costs.

A simple and reliable in vitro immunoresistant assay that could determine whether a patient is developing an immune response against a BONT or TeNT would be of significant value. First, an in vitro test of this nature would eliminate the inconvenience, discomfort and apprehension associated with patient-based assays. In addition, such an in vitro immunoresistant assay would alleviate the need for animal testing and all the disadvantages, costs and concerns associated with this type of assay. The present invention provides novel immunoresistant assays for determining the presence or absence of neutralizing anti-BoNT/A antibodies useful for various scientific, therapeutic, clinical and cosmetic applications, and provides related advantages as well.

### BRIEF DESCRIPTION OF THE DRAWINGS

FIG. 1 shows a schematic of the BoNT/A Immunoresistant Assay using a sample containing neutralizing anti-BoNT/A antibodies. This non-limiting example illustrates that neutralizing anti-BoNT/A antibodies will bind to the toxin, thereby reducing or preventing the formation of toxin-receptor complexes. The asterisks on the toxin represents either one of a variety of marker compounds suitable for a detection means selected or one of a variety of peptides suitable for a detection system selected as disclosed in the present specification.

FIG. 2 shows a schematic of the BoNT/A Immunoresistant Assay using a sample not containing neutralizing anti-BoNT/A antibodies. This non-limiting example illustrates that the absence of neutralizing anti-BoNT/A antibodies will allow the formation of toxin-receptor complexes. The asterisks on the toxin represents either one of a variety of marker compounds suitable for a detection means selected or one of a variety of peptides suitable for a detection system selected as disclosed in the present specification.

FIG. 3 shows a schematic of the BoNT/A Immunoresistant Assay using a BoNT/A toxin disclosed in the present specification attached to a solid support. This non-limiting example illustrates that neutralizing anti-BoNT/A antibodies will bind to the toxin, thereby reducing or preventing the formation of toxin-receptor complexes (left side of FIG. 3) or that the absence of neutralizing anti-BoNT/A antibodies will allow the formation of toxin-receptor complexes (right side of FIG. 3). The asterisks on the receptor represents either one of a variety of marker compounds suitable for a detection means selected or one of a variety of peptides suitable for a detection system selected as disclosed in the present specification. In an alternative protocol, free BoNT/A receptor can be detecting instead of toxin-receptor complexes.

FIG. 4 shows a schematic of the BoNT/A Immunoresistant Assay using a receptor system disclosed in the present specification attached to a solid support. This non-limiting example illustrates that neutralizing anti-BoNT/A antibodies will bind to the toxin, thereby reducing or preventing the formation of toxin-receptor complexes (left side of FIG. 4) or that the absence of neutralizing anti-BoNT/A antibodies will allow the formation of toxin-receptor complexes (right side of FIG. 4). The asterisks on the toxin represents either one of a variety of marker compounds suitable for a detection means selected or one of a variety of peptides suitable for a detection system selected as disclosed in the present specification. In an alternative protocol, BoNT/A toxin in the form of toxin-antibody complexes can be detecting instead of toxin-receptor complexes.

FIG. 5 shows a schematic of the current paradigm of neurotransmitter release and Clostridial toxin intoxication in a central and peripheral neuron. FIG. 1a shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1b shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including changes in the internal pH of the vesicle, formation of a channel pore comprising the H_{N} domain of Clostridial toxin heavy chain, separation of the Clostridial toxin light chain from the heavy chain, and release of the activate light chain into the cytosol and 4) enzymatic target modification, where the activate light chain of Clostridial toxin proteolytically cleaves its target SNARE substrates, such as, e.g., SNAP-25, VAMP or Syntaxin, thereby preventing vesicle docking and neurotransmitter release.

FIG. 6 shows saturation curve experiments of ¹²⁵I-labeled BoNT/A to mouse synaptosomes. The experiments were carried out using 50,000 counts/minute (about 1 ng) of ¹²⁵-labeled active BoNT/A toxin that was allowed to bind to different volumes of mouse a synaptosome preparation (from 0 to 8 μL).

FIG. 7 shows an inhibition of the binding of ¹²⁵I-labeled BoNT/A to mouse synaptosomes by unlabeled BoNT/A toxin (), or inactivate BoNT/A toxin (▪). The experiments were carried out using 50,000 counts/minute (about 1 ng) of ¹²⁵I-labeled active BoNT/A toxin that was allowed to bind to 4 μL of synaptosomes in the presence of different amounts of either unlabeled active BoNT/A toxin (), or inactivate BoNT/A toxin (▪). The levels of binding of ¹²⁵I-labeled toxin in the presence of different amounts of unlabeled toxin relative to the uninhibited controls were used to determine the percent of inhibition. The data are presented in percent binding in the presence of different concentrations of unlabeled BoNT/A (FIG. 7a) and as the percent inhibition values are plotted as a function of the reciprocal of inhibitor concentration (FIG. 7b).

FIG. 8 shows an inhibition of the binding of ¹²⁵I-labeled BoNT/A to mouse synaptosomes by unlabeled H_{N} and H_{C} BoNT/A toxin peptide fragments. The experiments were carried out using 50,000 counts/minute (about 1 ng) of ¹²⁵I-labeled active BoNT/A toxin peptide that was allowed to bind to 4 μL of synaptosomes in the presence of different amounts of individual unlabeled H_{N} and H_{C} BoNT/A toxin peptides. The levels of binding of ¹²⁵I-labeled toxin in the presence of different amounts of individual unlabeled H_{N} and H_{C} BoNT/A toxin peptides relative to the uninhibited controls were used to determine the percent of inhibition. The data are presented in percent binding in the presence of different concentrations of individual unlabeled H_{N} and H_{C} BoNT/A toxin peptides (FIG. 8a) and as the percent inhibition values are plotted as a function of the reciprocal of inhibitor concentration (FIG. 8b).

FIG. 9 shows the inhibition curves of the binding of ¹²⁵I-labeled BoNT/A to mouse synaptosomes by unlabeled H_{N} and H_{C} BoNT/A toxin peptide fragment mixtures. The experiments were carried out using 50,000 counts/minute (about 1 ng) of ¹²⁵I-labeled active BoNT/A toxin peptide that was allowed to bind to 4 μL of synaptosomes in the presence of different amounts of unlabeled H_{N} and H_{C} BoNT/A toxin peptides containing equimolar quantities of the following peptides: The six H_{N} peptides of amino acids 533 to 551 (N7) of SEQ ID NO: 1, 659 to 677 (N16) of SEQ ID NO: 1, 701 to 719 (N19) of SEQ ID NO: 1, 729 to 747 (N21) of SEQ ID NO: 1, 757 to 775 (N23) of SEQ ID NO: 1, 799 to 817 (N26) of SEQ ID NO: 1 (); The five H_{C} peptides of amino acids 1065 to 1083 (C16) of SEQ ID NO: 1, 1107 to 1125 (C19) of SEQ ID NO: 1, 1163 to 1181 (C23) of SEQ ID NO: 1, 1233 to 1251 (C28) of SEQ ID NO: 1 and 1275 to 1296 (C31) of SEQ ID NO: 1 (▪); and a mixture containing the six H_{N} peptides of and the five H_{C} peptides of amino acids 533 to 551 (N7) of SEQ ID NO: 1, 659 to 677 (N16) of SEQ ID NO: 1, 701 to 719 (N19) of SEQ ID NO: 1, 729 to 747 (N21) of SEQ ID NO: 1, 757 to 775 (N23) of SEQ ID NO: 1, 799 to 817 (N26) of SEQ ID NO: 1, 1065 to 1083 (C16) of SEQ ID NO: 1, 1107 to 1125 (C19) of SEQ ID NO: 1, 1163 to 1181 (C23) of SEQ ID NO: 1, 1233 to 1251 (C28) of SEQ ID NO: 1 and 1275 to 1296 (C31) of SEQ ID NO: 1 (▴). The levels of binding of ¹²⁵I-labeled toxin in the presence of different amounts of individual unlabeled H_{N} and H_{C} BoNT/A toxin peptides relative to the uninhibited controls were used to determine the percent of inhibition. The data are presented in percent binding in the presence of different concentrations of individual unlabeled H_{N} and H_{C} BoNT/A toxin peptides.

FIG. 10 shows the inhibition profile of the binding of ¹²⁵I-labeled BoNT/A to mouse synaptosomes by unlabeled H_{N} and H_{C} BoNT/A toxin peptide fragments. The experiments were carried out using 50,000 counts/minute (about 1 ng) of ¹²⁵I-labeled active BoNT/A toxin peptide that was allowed to bind to 4 μL of synaptosomes in the presence of 1.0 μg of an individual unlabeled H-chain BoNT/A toxin peptide. The levels of binding of ¹²⁵I-labeled toxin in the presence of an individual unlabeled H-chain BoNT/A toxin peptide relative to the uninhibited controls were used to determine the percent of inhibition. The figure shows the values of maximum inhibition of each of the 60H-chain peptides obtained by titrations. N1, amino acids 449 to 467 of SEQ ID NO: 1; N2, amino acids 463 to 481 of SEQ ID NO: 1; N3, amino acids 477 to 495 of SEQ ID NO: 1; N4, amino acids 491-509 of SEQ ID NO: 1; N5, amino acids 505 to 523 of SEQ ID NO: 1; N6, amino acids 519 to 537 of SEQ ID NO: 1; N7, amino acids 533 to 551 of SEQ ID NO: 1; N8, amino acids 547 to 565 of SEQ ID NO: 1; N9, amino acids 561 to 579 of SEQ ID NO: 1; N10, amino acids 575 to 593 of SEQ ID NO: 1; N11, amino acids 589 to 607 of SEQ ID NO: 1; N12, amino acids 603 to 621 of SEQ ID NO: 1; N13, amino acids 617 to 635 of SEQ ID NO: 1; N14, amino acids 631 to 649 of SEQ ID NO: 1; N15, amino acids 645 to 663 of SEQ ID NO: 1; N16, amino acids 659 to 677 of SEQ ID NO: 1; N17, amino acids 673 to 691 of SEQ ID NO: 1; N18, amino acids 687 to 705 of SEQ ID NO: 1; N19, amino acids 701 to 719 of SEQ ID NO: 1; N20, amino acids 715 to 733 of SEQ ID NO: 1; N21, amino acids 729 to 747 of SEQ ID NO: 1; N22, amino acids 743 to 761 of SEQ ID NO: 1; N23, amino acids 757 to 775 of SEQ ID NO: 1; N24, amino acids 771 to 789 of SEQ ID NO: 1; N25, amino acids 785 to 803 of SEQ ID NO: 1; N26, amino acids 799 to 817 of SEQ ID NO: 1; N27, amino acids 813 to 831 of SEQ ID NO: 1; N28, amino acids 827 to 845 of SEQ ID NO: 1; N29, amino acids 841 to 859 of SEQ ID NO: 1; C1, amino acids 855 to 873 of SEQ ID NO: 1; C2, amino acids 869 to 887 of SEQ ID NO: 1; C3, amino acids 883 to 901 of SEQ ID NO: 1; C4, amino acids 897 to 915 of SEQ ID NO: 1; C5, amino acids 911 to 929 of SEQ ID NO: 1; C6, amino acids 925 to 943 of SEQ ID NO: 1; C7, amino acids 939 to 957 of SEQ ID NO: 1; C8, amino acids 953 to 971 of SEQ ID NO: 1; C9, amino acids 967 to 985 of SEQ ID NO: 1; C10, amino acids 981 to 999 of SEQ ID NO: 1; C11, amino acids 995 to 1013 of SEQ ID NO: 1; C12, amino acids 1009 to 1027 of SEQ ID NO: 1; C13, amino acids 1023 to 1041 of SEQ ID NO: 1; C14, amino acids 1037 to 1055 of SEQ ID NO: 1; C15, amino acids 1051 to 1069 of SEQ ID NO: 1; C16, amino acids 1065 to 1083 of SEQ ID NO: 1; C17, amino acids 1079 to 1097 of SEQ ID NO: 1; C18, amino acids 1093 to 1111 of SEQ ID NO: 1; C19, amino acids 1107 to 1125 of SEQ ID NO: 1; C20, amino acids 1121 to 1139 of SEQ ID NO: 1; C21, amino acids 1135 to 1153 of SEQ ID NO: 1; C22, amino acids 1149 to 1167 of SEQ ID NO: 1; C23, amino acids 1163 to 1181 of SEQ ID NO: 1; C24, amino acids 1177 to 1195 of SEQ ID NO: 1; C25, amino acids 1191 to 1209 of SEQ ID NO: 1; C26, amino acids 1205 to 1223 of SEQ ID NO: 1; C27, amino acids 1219 to 1237 of SEQ ID NO: 1; C28, amino acids 1233 to 1251 of SEQ ID NO: 1; C29, amino acids 1247 to 1265 of SEQ ID NO: 1; C30, amino acids 1261 to 1279 of SEQ ID NO: 1; C31, amino acids 1275 to 1296 of SEQ ID NO: 1.

FIG. 11 shows a comparison of the H-chain peptides that bind mouse synaptosomes with those that bind blocking (i.e., protecting) mouse anti-BoNT/A antibodies. The results of the antibody binding profile was obtained from M. Zouhair Atassi & Behzod Z. Dolimbek, Mapping of the Antibody-Binding Regions on the H_{N}-Domain (Residues 449-859) of Botulinum Neurotoxin A with Antitoxin Antibodies from Four Host Species. Full Profile of the Continuous Antigenic Regions of the H-Chain of Botulinum Neurotoxin A, 23(1) PROTEIN J. 39-52 (2004).

FIG. 12 shows saturation curve experiments of ¹²⁵I-labeled active BoNT/A toxin () or ¹²⁵I-labeled inactivated BoNT/A toxin (▴) binding to a mouse synaptosome preparation using a mixture of sera from 5 and MPA-positive cervical dystonia patients or by a mixture of 5 normal sera (▪). The experiments were carried out using 50,000 counts/minute (about 1 ng) of either ¹²⁵I-labeled active BoNT/A toxin or ¹²⁵I-labeled inactive BoNT/A toxin that were allowed to bind to 4 μL of synaptosomes in the presence of different amounts of serum (from 0 to 2 μL).

FIG. 13 shows representative data from a BoNT/A Immunoresistant Assay using an ¹²⁵I-labeled active BoNT/A toxin. The experiments were carried out using 50,000 counts/minute (about 1 ng) of a ¹²⁵ I-labeled BoNT/A toxin that was mixed with 4 μL of a mouse synaptosome preparation in the presence of 1 μL of serum. The mixture of 5 normal sera was used as a negative control to establish a zero-point toxin-antibody complex formation reference. Group 1, MPA-positive sera obtained from cervical dystonia patients (n=30) who have become unresponsive to BoNT/A therapy and were determined to have high levels of anti-BoNT/A antibodies based on the MPA assay. Group 2, MPA-negative sera obtained from cervical dystonia patients (n=28) who were still responsive to BoNT/A therapy and were determined to have low levels of anti-BoNT/A antibodies based on the MPA assay. Group 3, control sera obtained from individuals (n=19) who were never administered a BoNT/A therapy.

FIG. 14 shows representative data from a BoNT/A Immunoresistant Assay using a ¹²⁵I-labeled inactive BoNT/A toxin. The experiments were carried out using 50,000 counts/minute (about 1 ng) of a ¹²⁵I-labeled inactive toxin that was mixed with 4 μL of a mouse synaptosome preparation in the presence of 1 μL of serum. The mixture of 5 normal sera was used as a negative control to establish a zero-point toxin-antibody complex formation reference. Group 1, MPA-positive sera obtained from cervical dystonia patients (n=30) who had become unresponsive to BoNT/A therapy and were determined to have high levels of anti-BoNT/A antibodies based on the MPA assay. Group 2, MPA-negative sera obtained from cervical dystonia patients (n=28) who were still responsive to BoNT/A therapy and were determined to have low levels of anti-BoNT/A antibodies based on the MPA assay. Group 3, control sera obtained from individuals (n=19) who were never administered a BoNT/A therapy.

FIG. 15 shows expression construct pET28a-His-BoNT/A (H227Y) encoding the inactive BoNT/A toxin of SEQ ID NO: 3 operably-linked to an amino-terminal polyhistidine peptide. A Thrombin protease cleavage site is operably-linked between the 6×His binding peptide and the BoNT/A (H227Y). Abbreviations are as follows: P_{T7}, a bacteriophage T7 promoter region; 6×His, a region encoding a polyhistidine peptide; Thrombin, a region encoding a thrombin cleavage site; BoNT/A (H227Y), the nucleic acid molecule of SEQ ID NO: 4 encoding the inactive BoNT/A (H227Y) of SEQ ID NO: 3; T7 TT, a bacteriophage T7 transcription termination region; f1 origin, a bacteriophage f1 origin of replication; Kanamycin, a region encoding an aminophosphotransferase that confers Kanamycin; pBR322 ori, a pBR322 plasmid origin of replication region; lacl, a region encoding a lactose 1.

FIG. 16 shows a plasmid map of bacterial expression construct pQB167-H_{C}BoNT/A-GFP encoding the H_{C}BoNT/A of SEQ ID NO: 7 operably-linked to a carboxyl-terminal GFP. Abbreviations are as follows: P_{T7}, a bacteriophage T7 promoter region; H_{C}BoNT/A, the nucleic acid composition of SEQ ID NO: 8 encoding the H_{C}BoNT/A of SEQ ID NO: 7; GFP, a region encoding a Green Florescence Protein; T7 TT, a bacteriophage T7 transcription termination region; Ampicillin, a region encoding a β-lactamase that confers Ampicillin resistance; pBR322 ori, a pBR322 origin of plasmid replication region; lacl, a region encoding a lactose 1.

FIG. 17 shows a plasmid map of mammalian expression construct pQBI 25-H_{C}BoNT/A-GFP encoding the H_{C}BoNT/A of SEQ ID NO: 7 operably-linked to a carboxyl-terminal GFP. Abbreviations are as follows: P_{CMV}, an cytomegalovirus promoter region; H_{C}BoNT/A, the nucleic acid molecule of, SEQ ID NO: 8 encoding the H_{C}BoNT/A of SEQ ID NO: 7; GFP, a region encoding a Green Florescence Protein; BGH pA, a bovine growth hormone polyadenylation site; Neomycin, a region encoding an aminophosphotransferase that confers Neomycin resistance; pUC ori, a pUC origin of plasmid replication region; Ampicillin, a region encoding a β-lactamase that confers Ampicillin resistance.

FIG. 18 shows a plasmid map of mammalian expression construct pRlucC-Luc-H_{C}BoNT/A-His encoding the H_{C}BoNT/A of SEQ ID NO: 7 operably-linked to an amino-terminal luciferase and a carboxyl-terminal polyhistidine peptide. Abbreviations are as follows: P_{CMV}, an cytomegalovirus promoter region; Rluc, a region encoding a luciferase; H_{C}BoNT/A, the nucleic acid molecule of SEQ ID NO: 8 encoding the H_{C}BoNT/A of SEQ ID NO: 7; 6×His, a nucleic acid composition of SEQ ID NO: 8 encoding a polyhistidine peptide of SEQ ID NO: 7; SV40 pA, a simian virus 40 polyadenylation site; f1 ori, a bacteriophage f1 origin of replication; Kanamycin/Neomycin, a region encoding an aminophosphotransferase that confers Kanamycin and Neomycin resistance; P_{SV40}/P_{AMP}, a simian virus 40 and β-lactamase promoter region; TK pA, a Thymidine Kinase polyadenylation site.

FIG. 19 shows a plasmid map of E. coli expression construct pET28a-His-FGFR3 encoding the FGFR3 of SEQ ID NO: 11 operably-linked to an amino-terminal polyhistidine peptide. A Thrombin protease cleavage site is operably-linked between the polyhistidine peptide and the FGFR3. Abbreviations are as follows: PT7, a bacteriophage T7 promoter region; 6×His, a region encoding a polyhistidine peptide; Thrombin, a region encoding a thrombin cleavage site; FGFR3, the nucleic acid molecule of SEQ ID NO: 12 encoding an the FGFR3 of SEQ ID NO: 11; T7 TT, a bacteriophage T7 transcription termination region; f1 origin, a bacteriophage f1 origin of replication; Kanamycin, a region encoding an aminophosphotransferase that confers Kanamycin; pBR322 ori, a pBR322 plasmid origin of replication region; lacl, a region encoding a lactose 1.

### DETAILED DESCRIPTION

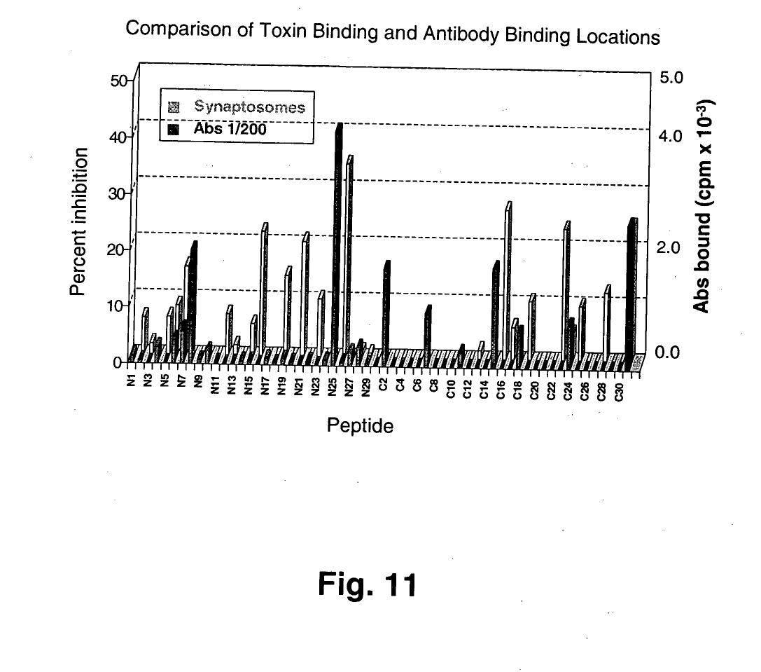

The present invention provides novel immunoresistant assays for determining the presence or absence of neutralizing anti-BoNT/A antibodies. The basis for the present invention takes advantage of the shared binding properties BoNT/A and neutralizing anti-BoNT/A antibodies have for the BoNT/A receptor system found on the surface of a target cell. Regions on the heavy chain necessary for the binding of BoNT/A toxin to its receptor were identified by synthesizing a panel of uniform-sized, overlapping peptides that encompass the entire 848-residue heavy chain and determining which of these peptides inhibited the binding of BoNT/A to mouse brain synaptosomes. In addition, this panel of heavy chain peptides to identify the heavy chain regions bound by neutralizing anti-BoNT/A antibodies (M. Zouhair Atassi et al., Mapping of the Antibody-binding Regions on Botulinum Neurotoxin H-chain Domain 855-1296 with Anti-toxin Antibodies from Three Host Species, 15 J. PROT. CHEM. 691-700, (1996); M. Zouhair Atassi & Behzod Z. Dolimbek, Mapping of the Antibody-binding Profile on Botulinum Neurotoxin A H_{N}-domain (residues 449-859) with Anti-toxin Antibodies from Four Host Species. Antigenic Profile of the Entire H-chain of Botulinum Neurotoxin A, 23(1) PROTEIN J. 39-52, (2004). Strikingly, comparisons of these heavy chain peptide-binding profiles showed that a number of the receptor-binding regions of BoNT/A overlapped significantly with the regions bound by neutralizing anti-BoNT/A antibody (FIG. 11). Since the regions of BoNT/A that bind the receptor overlap the regions recognized by the neutralizing antibodies, neutralizing anti-BoNT/A antibodies may function by preventing BoNT/A binding to its receptor. Therefore, the present invention exploits this discovery and provides methods and compositions for determining whether a given sample, such as, e.g., a human serum sample, contains neutralizing anti-BoNT/A antibodies by their ability to reduce or prevent BoNT/A binding to its receptor.

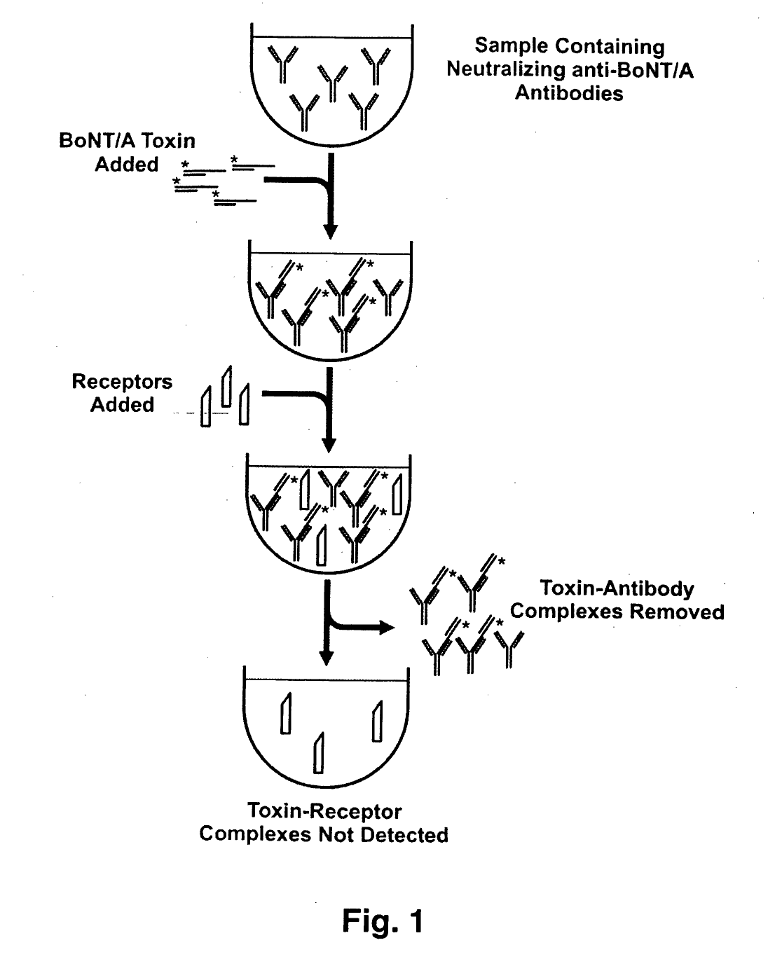

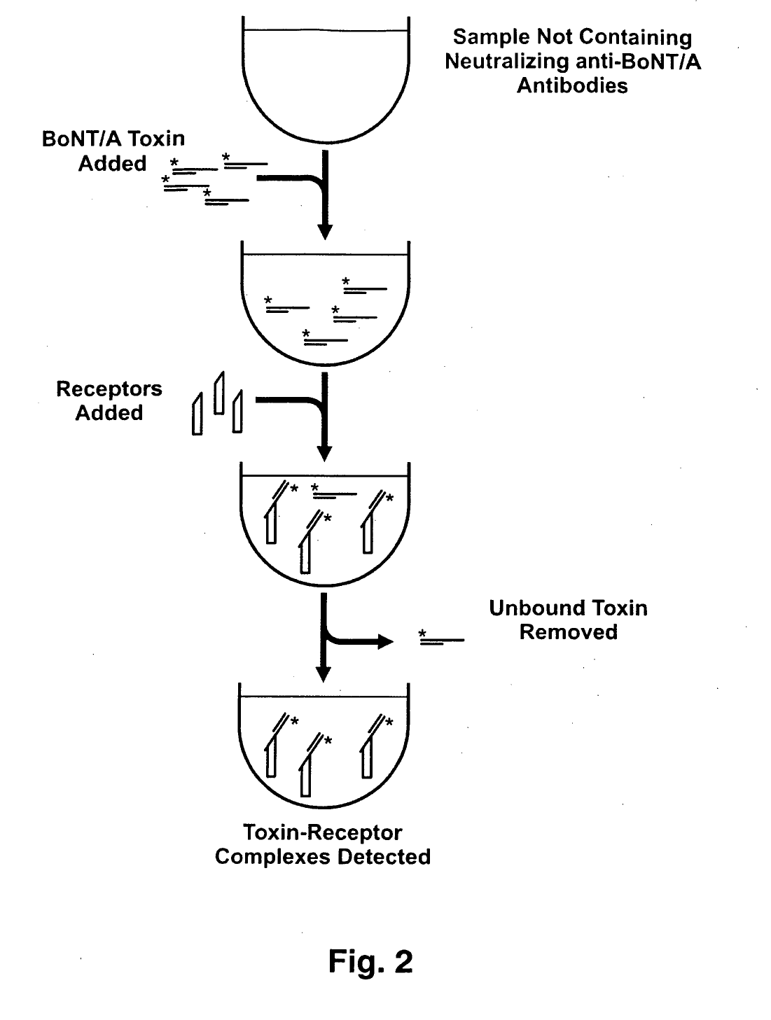

The general concept of the present invention is as follows. A BoNT/A is added to a sample that is being tested for the presence or absence of neutralizing anti-BoNT/A antibodies. If present in a test sample, the neutralizing anti-BoNT/A antibodies will make contact with the BoNT/A, forming an antibody-toxin complex (see, e.g., FIG. 1). A receptor capable of binding to a BoNT/A is next added to the sample. The presence of neutralizing anti-BoNT/A antibodies in a test sample will block the ability of the BoNT/A to bind to the receptor (see, e.g., FIG. 1). This is because the contact location of the antibodies significantly overlaps with the receptor binding region of the BoNT/A, thereby preventing the toxin from making contact with the receptor. However, if a test sample does not contain sufficient, if any, neutralizing anti-BoNT/A antibodies, then a BoNT/A can make contact with the receptor, forming a toxin-receptor complex (see, e.g., FIG. 2). The level of neutralizing anti-BoNT/A antibodies present in a test sample is determined by the amount of toxin-receptor complex formed. The formation of toxin-receptor complexes is inversely proportional to the level of neutralizing antibodies present in a test sample. Therefore, measurements revealing little or no formation of toxin-receptor complexes would indicate that a test sample contains high levels of neutralizing anti-BoNT/A antibodies (see, e.g., FIG. 1). Conversely, the formation of many toxin-receptor complexes would reveal a test sample containing little or no neutralizing anti-BoNT/A antibodies (see, e.g., FIG. 2).

Thus, aspects of the present invention provide methods of determining BoNT/A immunoresistance a mammal comprising the steps of adding a BoNT/A toxin and a BoNT/A receptor to a test sample and detecting the amount of toxin-receptor complexes formed by said toxin and said receptor.

Other aspects of the present invention provide methods of determining BoNT/A immunoresistance in a mammal comprising the steps of adding a BoNT/A toxin and a BoNT/A receptor to a test sample, detecting the amount of toxin-receptor complexes formed in said test sample, adding a BoNT/A toxin and a BoNT/A receptor to a control sample, detecting the amount of toxin-receptor complexes formed in said control sample and comparing the amount of toxin-receptor complexes formed in said test sample to the amount of toxin-receptor complexes formed in said control sample.

Other aspects of the present invention provide methods of determining BoNT/A immunoresistance in a mammal comprising the steps of adding a BoNT/A toxin to a test sample, contacting said toxin with a BoNT/A receptor and detecting the amount of toxin-receptor complexes formed by said toxin and said receptor.

Other aspects of the present invention provide methods of determining BoNT/A immunoresistance in a mammal comprising the steps of adding a BoNT/A toxin to a test sample, contacting said toxin with a BoNT/A receptor, detecting the amount of toxin-receptor complexes formed in said test sample, adding a BoNT/A toxin to a control sample, contacting said toxin with a BoNT/A receptor, detecting the amount of toxin-receptor complexes formed in said control sample, and comparing the amount of toxin-receptor complexes formed in said test sample relative to the amount of toxin-receptor complexes formed in said control sample.

Other aspects of the present invention provide methods of determining BoNT/A immunoresistance in a mammal comprising the steps of adding a BoNT/A toxin and a BoNT/A receptor to a test sample under conditions in which a neutralizing anti-BoNT/A antibody can bind said toxin to form a toxin-antibody complex and said toxin can bind said receptor to form a toxin-receptor complex; and detecting the presence or absence of one or more said toxin-receptor complexes; wherein the presence of said toxin-receptor complexes indicates the lack of BoNT/A immunoresistance and the absence of said toxin-receptor complexes indicates BoNT/A immunoresistance.

Other aspects of the present invention provide methods of determining BoNT/A immunoresistance in a mammal comprising the steps of adding a BoNT/A toxin to a test sample under conditions in which a neutralizing anti-BoNT/A antibody can bind said toxin to form a toxin-antibody complex; adding a BoNT/A receptor to said test sample under conditions in which said toxin can bind said receptor to form a toxin-receptor complex; and detecting the presence or absence of one or more said toxin-receptor complexes; wherein the presence of said toxin-receptor complexes indicates the lack of BoNT/A immunoresistance and the absence of said toxin-receptor complexes indicates BoNT/A immunoresistance.

Aspects of the present invention provide methods of determining BoNT/A immunoresistance in a test sample comprising the steps of adding a BoNT/A toxin and a BoNT/A receptor to said test sample under conditions in which a neutralizing anti-BoNT/A antibody can bind said toxin to form a toxin-antibody complex and said toxin can bind said receptor to form a toxin-receptor complex; and detecting the presence or absence of one or more said toxin-receptor complexes; wherein the presence of said BoNT/A-receptor complexes indicates the lack of BoNT/A immunoresistance and the absence of said BoNT/A-receptor complexes indicates BoNT/A immunoresistance.

Aspects of the present invention provide, in part, methods of determining BoNT/A immunoresistance in a test sample. As used herein, the term “BoNT/A immunoresistance” means an individual that does not fully respond to a BoNT/A therapy, or shows a reduced beneficial effect of a BoNT/A therapy because the immune response of that individual, either directly or indirectly, reduces the efficacy of the therapy. A non-limiting example of reduced efficacy would be the presence in an individual of at least one neutralizing anti-BoNT/A antibody that binds to a BoNT/A toxin in a manner that reduces or prevents the specificity or activity of the toxin. As used herein, the term “BoNT/A therapy” means a treatment, remedy, cure, healing, rehabilitation or any other means of counteracting something undesirable in a mammal requiring neuromodulation using a BoNT/A toxin or administering to a mammal one or more controlled doses of a medication, preparation or mixture of a BoNT/A toxin that has medicinal, therapeutic, curative, cosmetic, remedial or any other beneficial effect. BoNT/A therapy encompasses, without limitation, the use of any naturally occurring or modified fragment thereof, in any formulation, combined with any carrier or active ingredient and administered by any route of administration. An exemplary, well-known BoNT/A therapy is BOTOX® therapy.

Aspects of the present invention provide, in part, a sample. As used herein, the term “sample” means any biological matter that contains or potentially contains at least one anti-BoNT/A antibody. A anti-BoNT/A antibody can be a neutralizing anti-BoNT/A antibody or a non-neutralizing anti-BoNT/A antibody. As used herein, the term “neutralizing anti-BoNT/A antibodies” means any anti-BoNT/A antibody that will, under physiological conditions, bind to a region of a BoNT/A toxin in such a manner as to reduce or prevent the toxin from exerting its effect in a BoNT/A therapy. As used herein, the term “non-neutralizing anti-BoNT/A antibodies” means any anti-BoNT/A antibody that will, under physiological conditions, bind to a region of a BoNT/A toxin, but not prevent the toxin from exerting its effect in a BoNT/A therapy. It is envisioned that any and all samples that can contain anti-BoNT/A antibodies can be used in this method, including, without limitation, blood, plasma, serum and lymph fluid. In addition, any and all organisms capable of raising anti-BoNT/A antibodies against a BoNT/A toxin can serve as a source for a sample including, but not limited to, birds and mammals, including mice, rats, goats, sheep, horses, donkeys, cows, primates and humans. Non-limiting examples of specific protocols for blood collection and serum preparation are described in, e.g., Marjorie Schaub Di Lorenzo & Susan King Strasinger, BLOOD COLLECTION IN HEALTHCARE (F. A. Davis Company, 2001); and Diana Garza & Kathleen Becan-McBride, PHLEBOTOMY HANDBOOK: BLOOD COLLECTION ESSENTIALS (Prentice Hall, 6 ed., 2002). These protocols are routine procedures well within the scope of one skilled in the art and from the teaching herein.

A sample can be a test sample, or a sample can be a control sample. As used herein, the term “test sample” means any sample in which the presence or absence of a neutralizing anti-BoNT/A antibody is sought to be determined. A test sample can be obtained from an organism prior to exposure to a BoNT/A toxin, after a single BoNT/A treatment, after multiple BoNT/A toxin treatments, before onset of resistance to a BoNT/A therapy, or after onset of resistance to a BoNT/A therapy. As used herein, the term “control sample” means any sample in which the presence or absence of neutralizing anti-BoNT/A antibodies is known and includes both negative and positive control samples. A negative control sample can be obtained from an individual who was never exposed to BoNT/A toxin and may include, without limitation, a sample from the same individual supplying the test sample, but taken before undergoing a BoNT/A therapy; a sample taken from a different individual; a pooled sample taken from a plurality of different individuals. A positive control sample can be obtained from an individual manifesting BoNT/A immunoresistance and includes, without limitation, samples testing positive in a patient-based testing assays; samples testing positive in an in vivo bioassay; and samples showing hyperimmunity against an anti-BoNT/A antiserum.

It is further foreseen that anti-BoNT/A antibodies can be purified from a sample. Anti-BoNT/A antibodies can be purified from a sample, using a variety of procedures including, without limitation, Protein A/G chromatography and affinity chromatography. Non-limiting examples of specific protocols for purifying antibodies from a sample are described in, e.g., ANTIBODIES: A LABORATORY MANUAL (Edward Harlow & David Lane, eds., Cold Spring Harbor Laboratory Press, 2^{nd} ed. 1998); USING ANTIBODIES: A LABORATORY MANUAL: PORTABLE PROTOCOL No. I (Edward Harlow & David Lane, Cold Spring Harbor Laboratory Press, 1998); and MOLECULAR CLONING, A LABORATORY MANUAL, supra, (2001), which are hereby incorporated by reference. In addition, non-limiting examples of antibody purification methods as well as well-characterized reagents, conditions and protocols are readily available from commercial vendors that include, without limitation, Pierce Biotechnology, Inc., Rockford, Ill.; and Zymed Laboratories, Inc., South San Francisco, Calif. These protocols are routine procedures well within the scope of one skilled in the art.

Thus, an embodiment, the sample comprises blood. In aspect of this embodiment, the sample comprises mouse blood, rat blood, goat blood, sheep blood, horse blood, donkey blood, cow blood, primate blood and human blood. In another embodiment, the sample comprises plasma. In aspect of this embodiment, the sample comprises mouse plasma, rat plasma, goat plasma, sheep plasma, horse plasma, donkey plasma, cow plasma, primate plasma and human plasma. In another embodiment, the sample comprises serum. In aspect of this embodiment, the sample comprises mouse serum, rat serum, goat serum, sheep serum, horse serum, donkey serum, cow serum, primate serum and human serum. In another embodiment, the sample comprises lymph fluid. In aspect of this embodiment, the sample comprises mouse lymph fluid, rat lymph fluid, goat lymph fluid, sheep lymph fluid, horse lymph fluid, donkey lymph fluid, cow lymph fluid, primate lymph fluid and human lymph fluid. In yet another embodiment, the sample is a test sample. In yet another embodiment, the sample is a control sample.

BoNT/A is translated as a single chain polypeptide of approximately 150 kDa that is subsequently cleaved by proteolytic scission within a disulphide loop by bacterial or tissue proteases. This posttranslational processing yields a di-chain molecule comprising an approximately 50 kDa light chain (LC) and an approximately 100 kDa heavy chain (HC) held together by a single disulphide bond and noncovalent interactions. Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC(H_{N}) that facilitates release of the toxin from intracellular vesicles into the cytoplasm of the target cell; and 3) a binding domain found within the carboxyl-terminal half of the HC (H_{C}) that determines the binding activity and binding specificity of the toxin to the receptor complex located at the surface of the target cell.

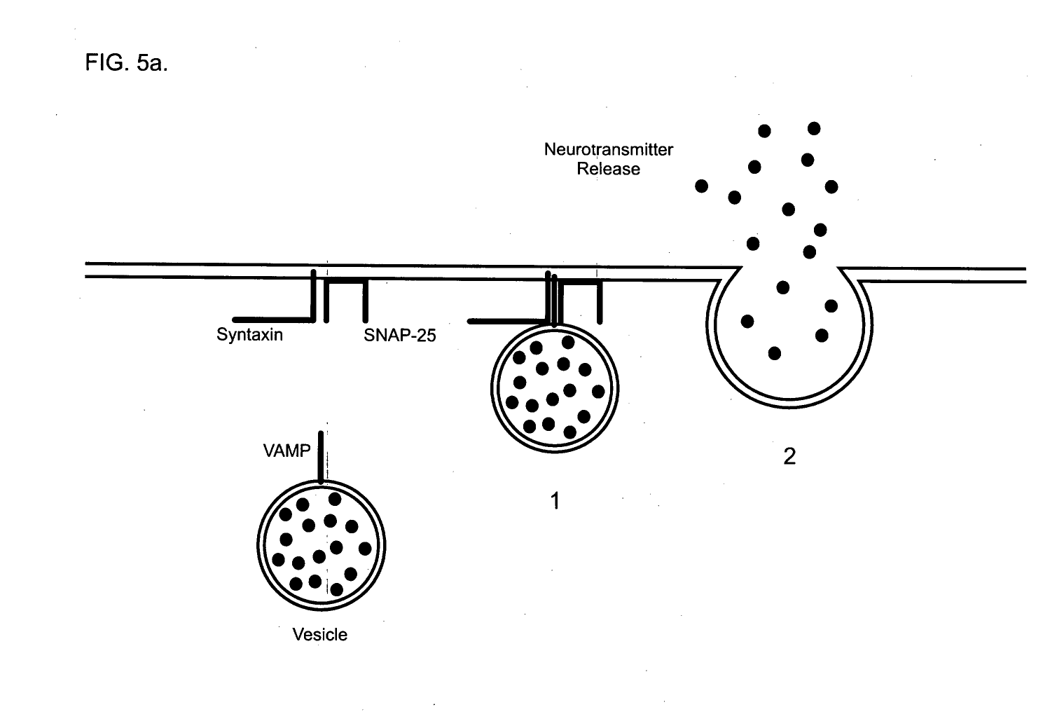

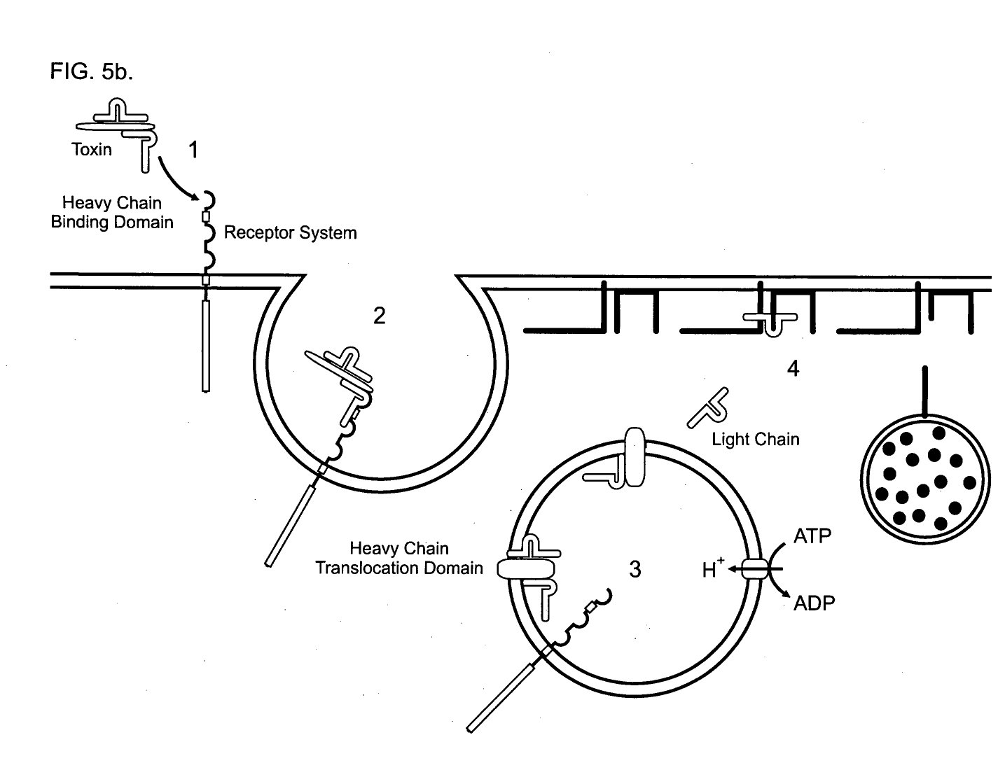

The binding, translocation and enzymatic activity of these three functional domains are all necessary for toxicity. While all details of this process are not yet precisely known, the overall cellular intoxication mechanism whereby BoNT/A enter a neuron and inhibit neurotransmifter release is similar, regardless of type. Although the applicants have no wish to be limited by the following description, the intoxication mechanism can be described as comprising at least four steps: 1) receptor binding, 2) complex internalization, 3) light chain translocation, and 4) enzymatic target modification (see FIG. 5). The process is initiated when the H_{C} domain of BoNT/A binds to a BoNT/A receptor complex located on the plasma membrane surface of a target cell. The binding specificity of the BoNT/A receptor complex is thought to be achieved, in part, by a specific combination of gangliosides and protein receptors that appear to distinctly comprise each Clostridial toxin receptor complex. Once bound, the toxin-receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote enzymatic activation of the toxin. Once activated, light chain endopeptidase of the toxin is released from the intracellular vesicle into the cytosol where it specifically targets one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A cleaves SNAP-25 in the carboxyl-terminal region, releasing a nine amino acid segment and can account for the total block of neurotransmitter release caused by BoNT/A in vivo.

Aspects of the present invention provide, in part, a BoNT/A toxin. As used herein, the term “BoNT/A toxin” means any BoNT/A toxin that retains the binding activity and binding specificity for its BoNT/A receptor and can also be bound by a neutralizing anti-BoNT/A antibody. As used herein, the term “binding activity” means that one molecule is directly or indirectly contacting another molecule via at least one intermolecular or intramolecular force, including, without limitation, a covalent bond, an ionic bond, a metallic bond, a hydrogen bond, a hydrophobic interaction, a van der Waals interaction, and the like, or any combination thereof. “Bound” and “bind” are considered terms for binding. As used herein, the term “binding specificity” means having a unique effect or influence or reacting in only one way or with only one thing. It is envisioned that any and all BoNT/A toxins that retain their binding activity and binding specificity for a BoNT/A receptor and can be bound by a neutralizing anti-BoNT/A antibody can be useful in a method disclosed in the present specification. Thus, BoNT/A toxin encompass, without limitation, an active BoNT/A toxin, an inactive BoNT/A toxin, a BoNT/A toxin di-chain molecule, a single-chain BoNT/A toxin molecule, a synthetic BoNT/A toxin molecule, a recombinant BoNT/A toxin molecule, a naturally occurring BoNT/A toxin variant, such as, e.g., a BoNT/A toxin isoform or a BoNT/A toxin subtype; a non-naturally occurring BoNT/A toxin variant, such as, e.g., a conservative BoNT/A toxin variant, a non-conservative BoNT/A toxin variant, a BoNT/A toxin peptidomimetic, a BoNT/A toxin chimeric variant and a BoNT/A toxin fragment, such as, e.g., a BoNT/A toxin heavy chain fragment or a BoNT/A toxin H_{C} fragment, or any combination thereof. In addition, a BoNT/A toxin encompass, without limitation, commercially available pharmaceutical compositions, such as, e.g., BOTOX® (Allergan, Inc., Irvine, Calif.), Dysport®/Reloxin®, (Beaufour Ipsen, Porton Down, England), Linurase® (Prollenium, Inc., Ontario, Canada), Neuronox® (Medy-Tox, Inc., Ochang-myeon, South Korea) BTX-A (Lanzhou Institute Biological Products, China).

As used herein, the term “BoNT/A toxin variant,” whether naturally-occurring or non-naturally-occurring, means a BoNT/A toxin that has at least one amino acid change from the corresponding region of SEQ ID NO: 1 and can be described in percent identity to the corresponding region of SEQ ID NO: 1. As a non-limiting example, a BoNT/A toxin variant comprising amino acids 1-1296 of SEQ ID NO: 1 will have at least one amino acid difference, such as, e.g., an amino acid substitution, deletion or addition, as compared to the amino acid region 1-1296 of SEQ ID NO: 1. As another non-limiting example, a BoNT/A toxin variant comprising amino acids 449-1296 of SEQ ID NO: 1 will have at least one amino acid difference, such as, e.g., an amino acid substitution, deletion or addition, as compared to the amino acid region 449-1296 of SEQ ID NO: 1. As yet another non-limiting example, a BoNT/A toxin variant comprising amino acids 861-1296 of SEQ ID NO: 1 will have at least one amino acid difference, such as, e.g., an amino acid substitution, deletion or addition, as compared to the amino acid region 861-1296 of SEQ ID NO: 1. Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: improving the sensitivity of progressive multiple sequence alignment through sequence weighting, position-specific gap penalties and weight matrix choice, 22(22) Nucleic Acids Research 4673-4680 (1994); and iterative refinement, see, e.g., Osamu Gotoh, Significant improvement in accuracy of multiple protein sequence alignments by iterative refinement as assessed by reference to structural alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-box: a fundamentally new algorithm for the simultaneous alignment of several protein sequences, 8(5) CABIOS 501-509 (1992); Gibbs sampling, see, e.g., C. E. Lawrence et al., Detecting subtle sequence signals: a gibbs sampling strategy for multiple alignment, 262(5131) Science 208-214 (1993); Align-M, see, e.g., lvo Van Walle et al., Align-m—a new algorithm for multiple alignment of highly divergent sequences, 20(9) Bioinformatics:1428-1435 (2004).

Hybrid methods combine functional aspects of both global and local alignment methods. Non-limiting methods include, e.g., segment-to-segment comparison, see, e.g., Burkhard Morgenstern et al., Multiple DNA and protein sequence alignment based on segment-to-segment comparison, 93(22) Proc. Natl. Acad. Sci. U.S.A. 12098-12103 (1996); T-Coffee, see, e.g., Cédric Notredame et al., T-Coffee: a novel algorithm for multiple sequence alignment, 302(1) J. Mol. Biol. 205-217 (2000); MUSCLE, see, e.g., Robert C. Edgar, MUSCLE: Multiple sequence alignment with high score accuracy and high throughput, 32(5) Nucleic Acids Res. 1792-1797 (2004); and DIALIGN-T, see, e.g., Amarendran R Subramanian et al., DIALIGN-T: An improved algorithm for segment-based multiple sequence alignment, 6(1) BMC Bioinformatics 66 (2005).

As used herein, the term “naturally occurring BoNT/A toxin variant” means any BoNT/A toxin produced without the aid of any human manipulation, including, without limitation, BoNT/A toxin isoforms produced from alternatively-spliced transcripts and BoNT/A toxin isoforms produced by spontaneous mutation. As used herein, the term “non-naturally occurring BoNT/A toxin variant” means any BoNT/A toxin produced with the aid of human manipulation, including, without limitation, a BoNT/A toxin produced by genetic engineering using random mutagenesis or rational designed and a BoNT/A toxin produced by chemical synthesis.

As used herein, the term “inactive BoNT/A toxin” means a BoNT/A toxin that is incapable of BoNT/A intoxication, but still retains the binding activity and binding specificity for a BoNT/A receptor and can also be bound by a neutralizing anti-BoNT/A antibody. As used herein, the term “intoxication” means the overall cellular mechanism whereby a BoNT/A toxin proteolytically cleaves a substrate and encompasses the binding of a BoNT/A toxin to a low or high affinity receptor, the internalization of the toxin/receptor complex, the translocation of the BoNT/A toxin light chain into the cytoplasm and the enzymatic target modification of a BoNT/A toxin substrate. An inactive BoNT/A toxin can function in substantially the same manner as the BoNT/A toxin on which the inactive BoNT/A toxin is based, and can be substituted for the BoNT/A toxin in any aspect of the present invention. It is foreseen that any and all processes that result in an inactive or non-toxic BoNT/A toxin can be used, including, without limitation, chemical treatment and recombinant methods, such as, e.g., in vitro mutagenesis. In an embodiment, an inactive BoNT/A toxin used in the methods disclosed in the specification is inactivated by an aldehyde fixation process. In an embodiment, an inactive BoNT/A toxin used in the methods disclosed in the specification is inactivated by an alcohol fixation process. In an embodiment, an inactive BoNT/A toxin used in the methods disclosed in the specification is inactivated by a recombinant process.

In yet another embodiment, an inactive BoNT/A toxin is produced when the nucleic acid molecule encoding an active BoNT/A toxin, is changed to encode a toxin no longer capable of intoxication upon expression, with the proviso that this inactive BoNT/A toxin retains the binding activity and binding specificity for a BoNT/A receptor and can also be bound by a neutralizing anti-BoNT/A antibody. Thus aspects of this embodiment may include changed nucleic acid molecules encoding an inactive BoNT/A toxin that lacks, e.g., enzymatic activity, translocation activity, internalization activity, such as, e.g., endocytosis, or any other ability of the toxin to enter the cell once bound to a receptor, and the like, or any combination thereof. Thus, in an aspect of this embodiment, at least one nucleotide of SEQ ID NO: 2 is changed that results in the lack of enzymatic activity of a BoNT/A toxin upon expression. In another aspect of this embodiment, a plurality of such nucleotide changes to SEQ ID NO: 2 results in a lack of the enzymatic activity of a BoNT/A toxin upon expression. In aspects of this embodiment, a nucleotide alteration results in a tyrosine replacing the histidine at position 223 of BoNT/A from SEQ ID NO: 1; a nucleotide alteration results in a glutamine replacing the glutamate at position 224 of BoNT/A toxin from SEQ ID NO: 1 or a nucleotide alteration results in a tyrosine replacing the histidine at position 227 of BoNT/A from SEQ ID NO: 1. In yet another aspect of this embodiment, at least one nucleotide of SEQ ID NO: 2 is changed that results in a disruption of the translocation activity of a BoNT/A toxin upon expression. In another aspect of this embodiment, a plurality of such nucleotide alterations to SEQ ID NO: 2 results in a disruption of the translocation activity of a BoNT/A toxin upon expression. In yet another aspect of this embodiment, at least one nucleotide of SEQ ID NO: 2 is altered that results in a disruption of the internalization activity of a BoNT/A toxin upon expression, such as, e.g., endocytosis, or any other ability of the toxin to enter the cell once bound with its natural receptor. In another aspect of this embodiment, a plurality of such nucleotide alterations to SEQ ID NO: 2 results in a disruption of the internalization activity of a BoNT/A toxin upon expression, such as, e.g., endocytosis, or any other ability of the toxin to enter the cell once bound with its natural receptor. In yet another aspect of the embodiment, at least one nucleotide to SEQ ID NO: 2 is altered that results in the disruption of the enzymatic activity, the translocation activity, internalization activity, or any combination thereof, of a BoNT/A toxin upon expression. In yet another aspect of the embodiment, a plurality of such nucleotide alterations to SEQ ID NO: 2 results in a disruption of the enzymatic activity, the translocation activity, internalization activity, or any combination thereof, of a BoNT/A toxin upon expression.

In another aspect of this embodiment, at least one nucleotide change is made to a nucleic acid molecule encoding an active BoNT/A toxin such that the toxin is no longer capable of intoxication upon expression, with the proviso that this inactive BoNT/A toxin retains the binding activity and binding specificity for its BoNT/A receptor and can be bound by a neutralizing anti-BoNT/A antibody. In another aspect of this embodiment, a plurality of nucleotide changes are made to a nucleic acid molecules encoding an active BoNT/A toxin such that the toxin is no longer capable of intoxication upon expression, with the proviso that this inactive BoNT/A toxin retains the binding activity and binding specificity for its BoNT/A receptor and can be bound by a neutralizing anti-BoNT/A antibody. Thus, aspects of this embodiment can include a nucleic acid molecule with, e.g., one or more changes, two or more changes, three or more changes, four or more changes, five or more changes, ten or more changes, or 20 or more changes. In aspects of this embodiment, a nucleic acid molecule encoding an inactive BoNT/A toxin can delete one or more nucleotides, two or more nucleotides, three or more nucleotides, four or more nucleotides, five or more nucleotides, ten or more nucleotides, 20 or more nucleotides, 30 or more nucleotides, 40 or nucleotides, 50 or more nucleotides, 100 or more nucleotides, 200 or more nucleotides, 300 or more nucleotides, 400 or more nucleotides, or 500 or more nucleotides from the BoNT/A toxin on which the inactive BoNT/A toxin is based. In other aspects of this embodiment, a nucleic acid molecule encoding an inactive BoNT/A toxin can add one or more nucleotides, two or more nucleotides, three or more nucleotides, four or more nucleotides, five or more nucleotides, ten or more nucleotides, 20 or more nucleotides, 30 or more nucleotides, 40 or nucleotides, 50 or more nucleotides, 100 or more nucleotides, 200 or more nucleotides, 300 or more nucleotides, 400 or more nucleotides, or 500 or more nucleotides to the BoNT/A toxin on which the inactive BoNT/A toxin is based. In still other aspects of this embodiment, a nucleic acid molecule encoding an inactive BoNT/A toxin may substitute one or more nucleotides, two or more nucleotides, three or more nucleotides, four or more nucleotides, five or more nucleotides, ten or more nucleotides, 20 or more nucleotides, 30 or more nucleotides, 40 or nucleotides, 50 or more nucleotides, 100 or more nucleotides, 200 or more nucleotides, 300 or more nucleotides, 400 or more nucleotides, or 500 or more nucleotides from the BoNT/A toxin on which the inactive BoNT/A toxin is based. In yet other aspects of this embodiment, a nucleic acid molecule encoding an inactive BoNT/A toxin can also substitute at least 10 contiguous nucleotides, at least 15 contiguous nucleotides, at least 20 contiguous nucleotides, or at least 25 contiguous nucleotides from the BoNT/A toxin on which the inactive BoNT/A toxin is based, that possess at least 50% nucleotide identity, at least 65% nucleotide identity, at least 75% nucleotide identity, at least 85% nucleotide identity or at least 95% nucleotide identity to the BoNT/A toxin on which the inactive BoNT/A toxin is based.

In yet another embodiment, an inactive BoNT/A toxin is produced when the active BoNT/A toxin is changed to a toxin no longer capable of intoxication, with the proviso that this inactive BoNT/A toxin retains the binding activity and binding specificity for a BoNT/A receptor and can also be bound by a neutralizing anti-BoNT/A antibody. Thus aspects of this embodiment may include an active BoNT/A toxin changed into an inactive BoNT/A toxin that lacks, e.g., enzymatic activity, translocation activity, internalization activity, such as, e.g., endocytosis, or any other ability of the toxin to enter the cell once bound to a receptor, and the like, or any combination thereof. In an aspect of this embodiment, at least one amino acid of SEQ ID NO: 1 is changed that results in the lack of enzymatic activity of a BoNT/A toxin. In another aspect of this embodiment, a plurality of such amino acid changes to SEQ ID NO: 1 results in a lack of the enzymatic activity of a BoNT/A toxin. In aspects of this embodiment, an amino acid alteration results in a tyrosine replacing the histidine at position 223 of BoNT/A from SEQ ID NO: 1; an amino acid alteration results in a glutamine replacing the glutamate at position 224 of BoNT/A toxin from SEQ ID NO: 1 or an amino acid alteration results in a tyrosine replacing the histidine at position 227 of BoNT/A from SEQ ID NO: 1. In yet another aspect of this embodiment, at least one amino acid of SEQ ID NO: 1 is changed that results in a disruption of the translocation activity of a BoNT/A toxin. In another aspect of this embodiment, a plurality of such amino acid alterations to SEQ ID NO: 1 result in a disruption of the translocation activity of a BoNT/A toxin upon expression. In yet another aspect of this embodiment, at least one amino acid of SEQ ID NO: 1 is altered that results in a disruption of the internalization activity of a BoNT/A toxin, such as, e.g., endocytosis, or any other ability of the toxin to enter the cell once bound with its natural receptor. In another aspect of this embodiment, a plurality of such amino acid alterations to SEQ ID NO: 2 result in a disruption of the internalization activity of a BoNT/A toxin, such as, e.g., endocytosis, or any other ability of the toxin to enter the cell once bound with its natural receptor. In yet another aspect of the embodiment, at least one amino acid of SEQ ID NO: 1 is altered that results in the disruption of the enzymatic activity, the translocation activity, internalization activity, or any combination thereof, of a BoNT/A toxin. In yet another aspect of the embodiment, a plurality of such amino acid alterations to SEQ ID NO: 2 result in a disruption of the enzymatic activity, the translocation activity, internalization activity, or any combination thereof, of a BoNT/A toxin.

In another aspect of this embodiment, at least one amino acid change is made to an active BoNT/A toxin such that the toxin is no longer capable of intoxication, with the proviso that this inactive BoNT/A toxin retains the binding activity and binding specificity for its BoNT/A receptor and can be bound by a neutralizing anti-BoNT/A antibody. In another aspect of this embodiment, a plurality of amino acid changes are made to an active BoNT/A toxin such that the toxin is no longer capable of intoxication, with the proviso that this inactive BoNT/A toxin retains the binding activity and binding specificity for its BoNT/A receptor and can be bound by a neutralizing anti-BoNT/A antibody. In aspects of this embodiment, an inactive BoNT/A toxin can delete one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the BoNT/A toxin on which the inactive BoNT/A toxin is based. In other aspects of this embodiment, an inactive BoNT/A toxin can add one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids to the BoNT/A toxin on which the inactive BoNT/A toxin is based. In still other aspects of this embodiment, an inactive BoNT/A toxin may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the BoNT/A toxin on which the inactive BoNT/A toxin is based. In yet other aspects of this embodiment, an inactive BoNT/A toxin can also substitute at least 10 contiguous amino acids, at least 15 contiguous amino acids, at least 20 contiguous amino acids, or at least 25 contiguous amino acids from the BoNT/A toxin on which the inactive BoNT/A toxin is based, that possess at least 50% amino acid identity, at least 65% amino acid identity, at least 75% amino acid identity, at least 85% amino acid identity or at least 95% amino acid identity to the BoNT/A toxin on which the inactive BoNT/A toxin is based.

Non-limiting examples of specific methods that can be used to change nucleotides of a nucleic acid composition encoding inactive BoNT/A toxin disclosed in the present specification are described in, see e.g., MOLECULAR CLONING, A LABORATORY MANUAL (Joseph Sambrook & David W. Russell eds., Cold Spring Harbor Laboratory Press, 3^{rd} ed. 2001) and CURRENT PROTOCOLS IN MOLECULAR BIOLOGY (Frederick M. Ausubel et al., eds. John Wiley & Sons, 2004), which are hereby incorporated by reference. In addition, non-limiting examples of in vitro mutagenesis kits, as well as well-characterized reagents, conditions and protocols are readily available from commercial vendors that include, without limitation, BD Biosciences-Clontech, Palo Alto, Calif.; Invitrogen, Inc, Carlsbad, Calif.; and Stratagene, La Jolla, Calif. These protocols are routine procedures well within the scope of one skilled in the art and from the teaching herein.

As used herein, the term “conservative BoNT/A toxin variant” means a BoNT/A toxin that has at least one amino acid substituted by another amino acid or an amino acid analog that has at least one property similar to that of the original amino acid. Examples of properties include, without limitation, similar size, topography, charge, hydrophobicity, hydrophilicity, lipophilicity covalant-bonding capacity, hydrogen-bonding capacity, a physicochemically property, of the like, or any combination thereof. A conservative BoNT/A toxin variant can function in substantially the same manner as the BoNT/A toxin on which the conservative BoNT/A toxin variant is based, and can be substituted for the BoNT/A toxin in any aspect of the present invention. A conservative BoNT/A toxin variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the BoNT/A toxin on which the conservative BoNT/A toxin variant is based. A conservative BoNT/A toxin variant can also substitute at least 10 contiguous amino acids, at least 15 contiguous amino acids, at least 20 contiguous amino acids, or at least 25 contiguous amino acids from the BoNT/A toxin on which the conservative BoNT/A toxin variant is based, that possess at least 50% amino acid identity, at least 65% amino acid identity, at least 75% amino acid identity, at least 85% amino acid identity or at least 95% amino acid identity to the BONT/A toxin on which the conservative BoNT/A toxin variant is based.

As used herein, the term “non-conservative BoNT/A toxin variant” means a BoNT/A toxin in which 1) at least one amino acid is deleted from the BoNT/A toxin on which the non-conservative BoNT/A toxin variant is based; 2) at least one amino acid added to the BoNT/A toxin on which the non-conservative BoNT/A toxin variant is based; or 3) at least one amino acid is substituted by another amino acid or an amino acid analog that does not share any property similar to that of the original amino acid. A non-conservative BoNT/A toxin variant can function in substantially the same manner as the BoNT/A toxin on which the non-conservative BoNT/A toxin variant is based, and can be substituted for the BoNT/A toxin in any aspect of the present invention. A non-conservative BoNT/A toxin variant can delete one or more amino acids, two or more amino acids, three or more amino acids, four or more amino acids, five or more amino acids, and ten or more amino acids from the BoNT/A toxin on which the non-conservative BoNT/A toxin variant is based. A non-conservative BoNT/A toxin variant can add one or more amino acids, two or more amino acids, three or more amino acids, four or more amino acids, five or more amino acids, and ten or more amino acids to the BoNT/A toxin on which the non-conservative BoNT/A toxin variant is based. A non-conservative BoNT/A toxin variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the BoNT/A toxin on which the non-conservative BoNT/A toxin variant is based. A non-conservative BoNT/A toxin variant can also substitute at least 10 contiguous amino acids, at least 15 contiguous amino acids, at least 20 contiguous amino acids, or at least 25 contiguous amino acids from the BoNT/A toxin on which the non-conservative BoNT/A toxin variant is based, that possess at least 50% amino acid identity, at least 65% amino acid identity, at least 75% amino acid identity, at least 85% amino acid identity or at least 95% amino acid identity to the BoNT/A toxin on which the non-conservative BoNT/A toxin variant is based.

Thus, in aspects of this embodiment, a BoNT/A toxin can be a BoNT/A toxin that retains the binding activity and binding specificity for a BoNT/A receptor and can also be bound by a neutralizing anti-BoNT/A antibody and has, e.g., at least 70% amino acid identity with the BoNT/A toxin of SEQ ID NO: 1, at least 75% amino acid identity with the BoNT/A toxin of SEQ ID NO: 1, at least 80% amino acid identity with the BoNT/A toxin of SEQ ID NO: 1, at least 85% amino acid identity with the BoNT/A toxin of SEQ ID NO: 1, at least 90% amino acid identity with the BoNT/A toxin of SEQ ID NO: 1 or at least 95% amino acid identity with the BoNT/A toxin of SEQ ID NO: 1. In other aspects of this embodiment, the BoNT/A toxin is a BoNT/A toxin that retains the binding activity and binding specificity for a BoNT/A receptor and can also be bound by a neutralizing anti-BoNT/A antibody and has, e.g., at most one, two, three, four, five, six, seven, eight, nine, or ten amino acid substitutions relative to the BoNT/A toxin of SEQ ID NO: 1.

As used herein, the term “BoNT/A toxin peptidomimetic” means a BoNT/A toxin that has at least one amino acid substituted by a non-natural oligomer that has at least one property similar to that of the first amino acid. Examples of properties include, without limitation, topography of a peptide primary structural element, functionality of a peptide primary structural element, topology of a peptide secondary structural element, functionality of a peptide secondary structural element, of the like, or any combination thereof. A BoNT/A toxin peptidomimetic can function in substantially the same manner as the BoNT/A toxin on which the BoNT/A toxin peptidomimetic is based, and can be substituted for the BoNT/A toxin in any aspect of the present invention. A BoNT/A toxin peptidomimetic may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the BoNT/A toxin on which the BoNT/A toxin peptidomimetic is based. A BoNT/A toxin peptidomimetic can also substitute at least 10 contiguous amino acids, at least 15 contiguous amino acids, at least 20 contiguous amino acids, or at least 25 contiguous amino acids from the BoNT/A toxin on which the BoNT/A toxin peptidomimetic is based, that possess at least 50% amino acid identity, at least 65% amino acid identity, at least 75% amino acid identity, at least 85% amino acid identity or at least 95% amino acid identity to the BoNT/A toxin on which the BoNT/A toxin peptidomimetic is based. For examples of peptidomimetic see, e.g., Amy S. Ripka & Daniel H. Rich, Peptidomimetic design, 2(4) CURR. OPIN. CHEM. BIOL. 441-452 (1998); and M. Angels Estiarte & Daniel H. Rich, Peptidomimetics for Drug Design, 803-861 (BURGER'S MEDICINAL CHEMISTRY AND DRUG DISCOVERY Vol. 1 PRINCIPLE AND PRACTICE, Donald J. Abraham ed., Wiley-Interscience, 6 ed 2003), which are hereby incorporated by reference.

As used herein, the term “BoNT/A toxin chimeric variant” means a molecule comprising at least a portion of a BoNT/A toxin and at least a portion of at least one other protein to form a BoNT/A toxin. Such BoNT/A toxin chimeric molecules are described in, e.g., Clifford C. Shone et al., Recombinant Toxin Fragments, U.S. Pat. No. 6,461,617 (Oct. 8, 2002); Keith A. Foster et al., Clostridial Toxin Derivatives Able To Modify Peripheral Sensory Afferent Functions, U.S. Pat. No. 6,395,513 (May 28, 2002); Wei-Jin Lin et al., Neurotoxins with Enhanced Target Specificity, US 2002/0137886 (Sep. 26, 2002); Keith A. Foster et al., Inhibition of Secretion from Non-neural Cells, US 2003/0180289 (Sep. 25, 2003); J. Oliver Dolly et al., Activatable Recombinant Neurotoxins, WO 2001/014570 (Mar. 1, 2001); Clifford C. Shone et al., Recombinant Toxin Fragments, WO 2004/024909 (Mar. 25, 2004); and Keith A. Foster et al., Re-targeted Toxin Conjugates, WO 2005/023309 (Mar. 17, 2005).

It is also envisioned that any of a variety of BoNT/A toxin fragments can be useful in aspects of the present invention with the proviso that these toxin fragments retain the binding activity and binding specificity for its BoNT/A receptor and can also be bound by a neutralizing anti-BoNT/A antibody. Thus, aspects of this embodiment, a BoNT/A toxin disclosed in the specification can be a BoNT/A toxin fragment of, e.g., at least 6 amino acids, at least 7 amino acids, at least 8 amino acids, at least 9 amino acids, at least 10 amino acids, at least 15 amino acids, at least 20 amino acids, at least 25 amino acids, at least 30 amino acids, at least 40 amino acids, at least 50 amino acids, at least 60 amino acids, at least 70 amino acids, at least 80 amino acids, at least 90 amino acids, at least 100 amino acids, at least 125 amino acids, at least 150 amino acids, at least 175 amino acids, at least 200 amino acids, at least 250 amino acids, at least 300 amino acids, at least 350 amino acids, at least 400 amino acids, at least 450 amino acids, at least 500 amino acids at least 600 amino acids, at least 700 amino acids, at least 800 amino acids, at least 900 amino acids, at least 1000 amino acids, at least 1100 amino acids and at least 1200 amino acids. In other aspects of this embodiment, a BoNT/A toxin disclosed in the specification can be a BoNT/A toxin fragment of, e.g., at most 6 amino acids, at most 7 amino acids, at most 8 amino acids, at most 9 amino acids, at most 10 amino acids, at most 15 amino acids, at most 20 amino acids, at most 25 amino acids, at most 30 amino acids, at most 40 amino acids, at most 50 amino acids, at most 60 amino acids, at most 70 amino acids, at most 80 amino acids, at most 90 amino acids, at most 100 amino acids, at most 125 amino acids, at most 150 amino acids, at most 175 amino acids, at most 200 amino acids, at most 250 amino acids, at most 300 amino acids, at most 350 amino acids, at most 400 amino acids, at most 450 amino acids, at most 500 amino acids, at most 600 amino acids, at most 700 amino acids, at most 800 amino acids, at most 900 amino acids, at most 1000 amino acids, at most 1100 amino acids and at most 1200 amino acids. In other aspects of this embodiment, a BoNT/A toxin fragment can have from five to fifty amino acids, from eight to fifty amino acids, from ten to fifty amino acids, from five to twenty amino acids, from eight to twenty amino acids, from ten to twenty amino acids, from twelve to twenty amino acids or from fifteen to twenty amino acids.

In one aspect of this embodiment, the BoNT/A toxin can be used. In another aspect of this embodiment, the BoNT/A toxin of SEQ ID NO: 1 is used. In another aspect of this embodiment, a BoNT/A toxin fragment can be the heavy chain of BoNT/A toxin. In yet another aspect of this embodiment, the BoNT/A toxin fragment comprises amino acids 449 to 1296 of SEQ ID NO: 1. In another aspect of this embodiment, a BoNT/A toxin fragment can be the H_{C} domain of a BoNT/A toxin. In yet another aspect of this embodiment, the BoNT/A toxin fragment comprises amino acids 861 to 1296 of SEQ ID NO: 1. In yet other aspects of this embodiment, a BoNT/A toxin fragment comprises the amino acids 449 to 467 of SEQ ID NO: 1; 463 to 481 of SEQ ID NO: 1, 477 to 495 of SEQ ID NO: 1, 491-509 of SEQ ID NO: 1, 505 to 523 of SEQ ID NO: 1, 519 to 537 of SEQ ID NO: 1, 533 to 551 of SEQ ID NO: 1, 547 to 565 of SEQ ID NO: 1, 561 to 579 of SEQ ID NO: 1, 575 to 593 of SEQ ID NO: 1, 589 to 607 of SEQ ID NO: 1, 603 to 621 of SEQ ID NO: 1, 617 to 635 of SEQ ID NO: 1, 631 to 649 of SEQ ID NO: 1, 645 to 663 of SEQ ID NO: 1, 659 to 677 of SEQ ID NO: 1, 673 to 691 of SEQ ID NO: 1, 687 to 705 of SEQ ID NO: 1, 701 to 719 of SEQ ID NO: 1, 715 to 733 of SEQ ID NO: 1, 729 to 747 of SEQ ID NO: 1, 743 to 761 of SEQ ID NO: 1, 757 to 775 of SEQ ID NO: 1, 771 to 789 of SEQ ID NO: 1, 785 to 803 of SEQ ID NO: 1, 799 to 817 of SEQ ID NO: 1, 813 to 831 of SEQ ID NO: 1, 827 to 845 of SEQ ID NO: 1, 841 to 859 of SEQ ID NO: 1, 855 to 873 of SEQ ID NO: 1, 869 to 887 of SEQ ID NO: 1, 883 to 901 of SEQ ID NO: 1, 897 to 915 of SEQ ID NO: 1, 911 to 929 of SEQ ID NO: 1, 925 to 943 of SEQ ID NO: 1, 939 to 957 of SEQ ID NO: 1, 953 to 971 of SEQ ID NO: 1, 967 to 985 of SEQ ID NO: 1, 981 to 999 of SEQ ID NO: 1, 995 to 1013 of SEQ ID NO: 1, 1009 to 1027 of SEQ ID NO: 1, 1023 to 1041 of SEQ ID NO: 1, 1037 to 1055 of SEQ ID NO: 1, 1051 to 1069 of SEQ ID NO: 1, 1065 to 1083 of SEQ ID NO: 1, 1079 to 1097 of SEQ ID NO: 1, 1093 to 1111 of SEQ ID NO: 1, 1107 to 1125 of SEQ ID NO: 1, 1121 to 1139 of SEQ ID NO: 1, 1135 to 1153 of SEQ ID NO: 1, 1149 to 1167 of SEQ ID NO: 1, 1163 to 1181 of SEQ ID NO: 1, 1177 to 1195 of SEQ ID NO: 1, 1191 to 1209 of SEQ ID NO: 1, 1205 to 1223 of SEQ ID NO: 1, 1219 to 1237 of SEQ ID NO: 1, 1233 to 1251 of SEQ ID NO: 1, 1247 to 1265 of SEQ ID NO: 1, 1261 to 1279 of SEQ ID NO: 1, 1275 to 1296 of SEQ ID NO: 1, and the like, or any combination thereof.

In still another aspect of this embodiment, at least one BoNT/A toxin fragment can be used in a method disclosed in the specification. In still another aspect of this embodiment, a plurality of BoNT/A toxin fragments can be used in a method disclosed in the specification. Thus, aspect of this embodiment can include one or more BoNT/A toxin fragments selected from the following amino acids compositions: 449 to 467 of SEQ ID NO: 1, 463 to 481 of SEQ ID NO: 1, 477 to 495 of SEQ ID NO: 1, 491-509 of SEQ ID NO: 1, 505 to 523 of SEQ ID NO: 1, 519 to 537 of SEQ ID NO: 1, 533 to 551 of SEQ ID NO: 1, 547 to 565 of SEQ ID NO: 1, 561 to 579 of SEQ ID NO: 1, 575 to 593 of SEQ ID NO: 1, 589 to 607 of SEQ ID NO: 1, 603 to 621 of SEQ ID NO: 1, 617 to 635 of SEQ ID NO: 1, 631 to 649 of SEQ ID NO: 1, 645 to 663 of SEQ ID NO: 1, 659 to 677 of SEQ ID NO: 1, 673 to 691 of SEQ ID NO: 1, 687 to 705 of SEQ ID NO: 1, 701 to 719 of SEQ ID NO: 1, 715 to 733 of SEQ ID NO: 1, 729 to 747 of SEQ ID NO: 1, 743 to 761 of SEQ ID NO: 1, 757 to 775 of SEQ ID NO: 1, 771 to 789 of SEQ ID NO: 1, 785 to 803 of SEQ ID NO: 1, 799 to 817 of SEQ ID NO: 1, 813 to 831 of SEQ ID NO: 1, 827 to 845 of SEQ ID NO: 1, 841 to 859 of SEQ ID NO: 1, 855 to 873 of SEQ ID NO: 1, 869 to 887 of SEQ ID NO: 1, 883 to 901 of SEQ ID NO: 1, 897 to 915 of SEQ ID NO: 1, 911 to 929 of SEQ ID NO: 1, 925 to 943 of SEQ ID NO: 1, 939 to 957 of SEQ ID NO: 1, 953 to 971 of SEQ ID NO: 1, 967 to 985 of SEQ ID NO: 1, 981 to 999 of SEQ ID NO: 1, 995 to 1013 of SEQ ID NO: 1, 1009 to 1027 of SEQ ID NO: 1, 1023 to 1041 of SEQ ID NO: 1, 1037 to 1055 of SEQ ID NO: 1, 1051 to 1069 of SEQ ID NO: 1, 1065 to 1083 of SEQ ID NO: 1, 1079 to 1097 of SEQ ID NO: 1, 1093 to 1111 of SEQ ID NO: 1, 1107 to 1125 of SEQ ID NO: 1, 1121 to 1139 of SEQ ID NO: 1, 1135 to 1153 of SEQ ID NO: 1, 1149 to 1167 of SEQ ID NO: 1, 1163 to 1181 of SEQ ID NO: 1, 1177 to 1195 of SEQ ID NO: 1, 1191 to 1209 of SEQ ID NO: 1, 1205 to 1223 of SEQ ID NO: 1, 1219 to 1237 of SEQ ID NO: 1, 1233 to 1251 of SEQ ID NO: 1, 1247 to 1265 of SEQ ID NO: 1, 1261 to 1279 of SEQ ID NO: 1 or 1275 to 1296 of SEQ ID NO: 1.

In other aspects of this embodiment, a method can include one or more BoNT/A toxin fragments selected from the following amino acids compositions: 463 to 481 of SEQ ID NO: 1, 505 to 523 of SEQ ID NO: 1, 519 to 537 of SEQ ID NO: 1, 533 to 551 of SEQ ID NO: 1, 603 to 621 of SEQ ID NO: 1, 645 to 663 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1079 to 1097 of SEQ ID NO: 1, 1107 to 1125 of SEQ ID NO: 1, 1163 to 1181 of SE ID NO: 1, 1177 to 1195 of SEQ ID NO: 1, 1191 to 1209 of SEQ ID NO: 1, 1233 to 1251 of SEQ ID NO: 1 or 1275 to 1296 of SEQ ID NO: 1. In yet other aspects of this embodiment, a method can include one or more BoNT/A toxin fragments selected from the following amino acids compositions: 533 to 551 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1107 to 1125 of SEQ ID NO: 1, 1163 to 1181 of SEQ ID NO: 1, 1191 to 1209 of SEQ ID NO: 1, 1233 to 1251 of SEQ ID NO: 1 and 1275 to 1296 of SEQ ID NO: 1. In still other aspects of this embodiment, a method can include one or more BoNT/A toxin fragments selected from the following amino acids compositions: 659 to 677 of SEQ ID NO: 1, 729 to 747 of SEQ ID NO: 1, 799 to 817 of SEQ ID NO: 1, 1065 to 1083 of SEQ ID NO: 1, 1163 to 1181 of SEQ ID NO: 1, and 1275 to 1296 of SEQ ID NO: 1.

Aspects of the present invention provide, in part, a BoNT/A receptor. As used herein, the term “BoNT/A receptor” means any molecule or composition that retains the binding activity and binding specificity for a BoNT/A toxin. It is envisioned that any and all BoNT/A receptors capable of binding a BoNT/A toxin disclosed in the present specification, but not capable of binding a BoNT/A toxin bound to a neutralizing anti-BoNT/A antibody, can be used in methods disclosed in the present specification. Thus, a BoNT/A receptor encompasses, without limitation, a naturally occurring BoNT/A receptor, a naturally occurring BoNT/A receptor variant, such as, e.g., a BoNT/A receptor isoform or a BoNT/A receptor subtype; a non-naturally occurring BoNT/A receptor variant, such as, e.g., a conservative BoNT/A receptor variant, a non-conservative BoNT/A receptor variant, a BoNT/A receptor peptidomimetic, a synthetic BoNT/A receptor, a recombinant BoNT/A receptor, a BoNT/A receptor chimeric variant and a BoNT/A receptor fragment of any length, with the proviso that this fragment can bind a BoNT/A toxin, but can not bind a BoNT/A toxin bound to an anti-BoNT/A antibody or any combination thereof. Non-limiting examples of BoNT/A receptors include, e.g., fibroblast growth factor 3 receptor (FGFR3) and members of the synaptotagmin family.

BoNT/A toxin targets neurons via high-affinity interactions with receptor complexes. A native receptor complex comprises a class of complex glycosphingolipids called gangiolsides, such as, e.g. members of the G1b series (e.g., GD1b, GT1b and Gq1b), which appears to be involved in the general adherence of a BoNT to the cell membrane, and protein receptors that are believed to confer binding specificity for the toxin and mediate internalization of the toxin see, e.g., Humeau, supra, 2000; Turton, supra, 2002; Atassi, supra, 2003; Lalli, supra, 2003. Recently, the Fibroblast Growth Factor Receptor 3 (FGFR3) has be shown to bind BoNT/A, see, e.g., Ester Fernandez-Salas et al., Screening Methods and Compositions, U.S. Patent Application No. 60/547,591 (Feb. 24, 2004); and Ester Fernandez-Salas et al., Screening Methods and Compositions, PCT Patent Application No. 2005/006421 (Feb. 23, 2005), which are hereby incorporated in their entirety by reference. As used herein, the term “Fibroblast Growth Factor 3 Receptor” is synonymous with “FGFR3” and means a FGFR3 molecule or composition comprising a FGFR3 or a FGFR3-binding portion thereof that retains the binding activity and binding specificity for a BoNT/A toxin. A FGFR3 useful in the invention encompass, without limitation, a naturally occurring FGFR3, a naturally occurring FGFR3 variant, a non-naturally FGFR3 variant, such as, e.g., genetically engineered variants produced by random mutagenesis or rational designed, and active fragments derived from a FGFR3. It is envisioned that any and all FGFR3s capable of binding a BoNT/A toxin disclosed in the present specification, but not capable of binding a BoNT/A toxin bound to a neutralizing anti-BoNT/A antibody, can be used in methods disclosed in the present specification. Thus, a FGFR3 encompasses, without limitation, a naturally occurring FGFR3, a naturally occurring FGFR3 variant, such as, e.g., a FGFR3 isoform or a FGFR3 subtype; a non-naturally occurring FGFR3 variant, such as, e.g., a conservative FGFR3 variant, a non-conservative FGFR3 variant, a FGFR3 peptidomimetic, a synthetic FGFR3, a recombinant FGFR3, a FGFR3 chimeric variant and a FGFR3 fragment of any length, with the proviso that this fragment can bind a BoNT/A toxin, but can not bind a BoNT/A toxin bound to an anti-BoNT/A antibody or any combination thereof.

As used herein, the term “conservative FGFR3 variant” means a FGFR3 that has at least one amino acid substituted by another amino acid or an amino acid analog that has at least one property similar to that of the original amino acid. Examples of properties include, without limitation, similar size, topography, charge, hydrophobicity, hydrophilicity, lipophilicity covalant-bonding capacity, hydrogen-bonding capacity, a physicochemically property, of the like, or any combination thereof. A conservative FGFR3 variant can function in substantially the same manner as the FGFR3 on which the conservative FGFR3 variant is based, and can be substituted for the FGFR3 in any aspect of the present invention. A conservative FGFR3 variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the FGFR3 on which the conservative FGFR3 variant is based. A conservative FGFR3 variant can also substitute at least 10 contiguous amino acids, at least 15 contiguous amino acids, at least 20 contiguous amino acids, or at least 25 contiguous amino acids from the FGFR3 on which the conservative FGFR3 variant is based, that possess at least 50% amino acid identity, at least 65% amino acid identity, at least 75% amino acid identity, at least 85% amino acid identity or at least 95% amino acid identity to the FGFR3 on which the conservative FGFR3 variant is based.

As used herein, the term “non-conservative FGFR3 variant” means a FGFR3 in which 1) at least one amino acid is deleted from the FGFR3 on which the non-conservative FGFR3 variant is based; 2) at least one amino acid added to the FGFR3 on which the non-conservative FGFR3 variant is based; or 3) at least one amino acid is substituted by another amino acid or an amino acid analog that does not share any property similar to that of the original amino acid. A non-conservative FGFR3 variant can function in substantially the same manner as the FGFR3 on which the non-conservative FGFR3 variant is based, and can be substituted for the FGFR3 in any aspect of the present invention. A non-conservative FGFR3 variant can delete one or more amino acids, two or more amino acids, three or more amino acids, four or more amino acids, five or more amino acids, and ten or more amino acids from the FGFR3 on which the non-conservative FGFR3 variant is based. A non-conservative FGFR3 variant can add one or more amino acids, two or more amino acids, three or more amino acids, four or more amino acids, five or more amino acids, and ten or more amino acids to the FGFR3 on which the non-conservative FGFR3 variant is based. A non-conservative FGFR3 variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the FGFR3 on which the non-conservative FGFR3 variant is based. A non-conservative FGFR3 variant can also substitute at least 10 contiguous amino acids, at least 15 contiguous amino acids, at least 20 contiguous amino acids, or at least 25 contiguous amino acids from the FGFR3 on which the non-conservative FGFR3 variant is based, that possess at least 50% amino acid identity, at least 65% amino acid identity, at least 75% amino acid identity, at least 85% amino acid identity or at least 95% amino acid identity to the FGFR3 on which the non-conservative FGFR3 variant is based.

As used herein, the term “FGFR3 peptidomimetic” means a FGFR3 that has at least one amino acid substituted by a non-natural oligomer that has at least one property similar to that of the first amino acid. Examples of properties include, without limitation, topography of a peptide primary structural element, functionality of a peptide primary structural element, topology of a peptide secondary structural element, functionality of a peptide secondary structural element, of the like, or any combination thereof. A FGFR3 peptidomimetic can function in substantially the same manner as the FGFR3 on which the FGFR3 peptidomimetic is based, and can be substituted for the FGFR3 in any aspect of the present invention. A FGFR3 peptidomimetic may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the FGFR3 on which the FGFR3 peptidomimetic is based. A FGFR3 peptidomimetic can also substitute at least 10 contiguous amino acids, at least 15 contiguous amino acids, at least 20 contiguous amino acids, or at least 25 contiguous amino acids from the FGFR3 on which the FGFR3 peptidomimetic is based, that possess at least 50% amino acid identity, at least 65% amino acid identity, at least 75% amino acid identity, at least 85% amino acid identity or at least 95% amino acid identity to the FGFR3 on which the FGFR3 peptidomimetic is based. For examples of peptidomimetic see, e.g., Amy S. Ripka & Daniel H. Rich, Peptidomimetic design, 2(4) CURR. OPIN. CHEM. BIOL. 441-452 (1998); and M. Angels Estiarte & Daniel H. Rich, Peptidomimetics for Drug Design, 803-861 (BURGER'S MEDICINAL CHEMISTRY AND DRUG DISCOVERY Vol. 1 PRINCIPLE AND PRACTICE, Donald J. Abraham ed., Wiley-Interscience, 6^{th} ed 2003), which are hereby incorporated by reference.

It is also envisioned that any of a variety of FGFR3 fragments can be useful in aspects of the present invention with the proviso that these active fragments retain the binding activity and binding specificity for a BoNT/A toxin. Thus, aspects of this embodiment, a FGFR3 disclosed in the specification can be a FGFR3 fragment of, e.g., at least 20 amino acids, at least 25 amino acids, at least 30 amino acids, at least 40 amino acids, at least 50 amino acids, at least 60 amino acids, at least 70 amino acids, at least 80 amino acids, at least 90 amino acids, at least 100 amino acids, at least 125 amino acids, at least 150 amino acids, at least 175 amino acids, at least 200 amino acids, at least 250 amino acids, at least 300 amino acids, at least 350 amino acids, at least 400 amino acids, at least 450 amino acids, at least 500 amino acids at least 600 amino acids, at least 700 amino acids, at least 800 amino acids, at least 900 amino acids, at least 1000 amino acids, at least 1100 amino acids and at least 1200 amino acids. In other aspects of this embodiment, a FGFR3 disclosed in the specification can be a FGFR3 fragment of, e.g., at most 25 amino acids, at most 30 amino acids, at most 40 amino acids, at most 50 amino acids, at most 60 amino acids, at most 70 amino acids, at most 80 amino acids, at most 90 amino acids, at most 100 amino acids, at most 125 amino acids, at most 150 amino acids, at most 175 amino acids, at most 200 amino acids, at most 250 amino acids, at most 300 amino acids, at most 350 amino acids, at most 400 amino acids, at most 450 amino acids, at most 500 amino acids, at most 600 amino acids, at most 700 amino acids, at most 800 amino acids, at most 900 amino acids, at most 1000 amino acids, at most 1100 amino acids and at most 1200 amino acids.

As a non-limiting example, a human FGFR3, naturally occurring human FGFR3 variants, non-naturally human FGFR3 variants, and human FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a bovine FGFR3, naturally occurring bovine FGFR3 variants, non-naturally bovine FGFR3 variants, and bovine FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a rat FGFR3, naturally occurring rat FGFR3 variants, non-naturally rat FGFR3 variants, and rat FGFR3 fragments that retain the ability to bind a BoNT/A toxin, but can not bind a BoNT/A toxin bound to an anti-BoNT/A antibody, can be useful as a BoNT/A receptor in aspects of the present invention. In still another non-limiting example, a mouse FGFR3, naturally occurring mouse FGFR3 variants, non-naturally mouse FGFR3 variants, and mouse FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a chicken FGFR3, naturally occurring chicken FGFR3 variants, non-naturally chicken FGFR3 variants, and chicken FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a frog FGFR3, naturally occurring frog FGFR3 variants, non-naturally frog FGFR3 variants, and frog FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a newt FGFR3, naturally occurring newt FGFR3 variants, non-naturally newt FGFR3 variants, and newt FGFR3 fragments that retain the ability to bind a BoNT/A toxin, but can not bind a BoNT/A toxin bound to an anti-BoNT/A antibody, can be useful as a BoNT/A receptor in aspects of the present invention. In another non-limiting example, a zebrafish FGFR3, naturally occurring zebrafish FGFR3 variants, non-naturally zebrafish FGFR3 variants, and zebrafish FGFR3 fragments that retain the ability to bind a BoNT/A toxin, but can not bind a BoNT/A toxin bound to an anti-BoNT/A antibody, can be useful as a BoNT/A receptor in aspects of the present invention. In is also understood that both nucleic acid molecules, such as, e.g., DNA and RNA, that encode a FGFR3 disclosed in the present specification and peptide molecules or peptidomimetics comprising a FGFR3 disclosed in the present specification are useful in aspects of the present invention. SEQ ID NO: 11, 13, 15, 17, 19, 21, 23, 25, 27, 29, 31, 33, 35 and 37 disclose FGFR3s useful in aspects on the present invention which are encoded by the nucleic acid molecules of SEQ ID NO: 12, 14, 16, 18, 20, 22, 24, 26, 28, 30, 32, 34, 36 and 38, respectively.

Thus, in aspects of this embodiment, the FGFR3 can be a human FGFR3IIIb that selectively binds BoNT/A which has, e.g., at least 70% amino acid identity with the FGFR3 of SEQ ID NO: 11, at least 75% amino acid identity with the FGFR3 of SEQ ID NO: 11, at least 80% amino acid identity with the FGFR3 of SEQ ID NO: 11, at least 85% amino acid identity with the FGFR3 of SEQ ID NO: 11, at least 90% amino acid identity with the FGFR3 of SEQ ID NO: 11 or at least 95% amino acid identity with the FGFR3 of SEQ ID NO: 11. In other aspects of this embodiment, the FGFR3 is a human FGFR3IIIb that that selectively binds BoNT/A which has, e.g., at most one, two, three, four, five, six, seven, eight, nine, or ten amino acid substitutions relative to the FGFR3 of SEQ ID NO: 11.

In other aspects of this embodiment, the FGFR3 can be a human FGFR3IIIc that selectively binds BoNT/A which has, e.g., at least 70% amino acid identity with the FGFR3 of SEQ ID NO: 13, at least 75% amino acid identity with the FGFR3 of SEQ ID NO: 13, at least 80% amino acid identity with the FGFR3 of SEQ ID NO: 13, at least 85% amino acid identity with the FGFR3 of SEQ ID NO: 13, at least 90% amino acid identity with the FGFR3 of SEQ ID NO: 13 or at least 95% amino acid identity with the FGFR3 of SEQ ID NO: 13. In other aspects of this embodiment, the FGFR3 is a human FGFR3IIIc that that selectively binds BoNT/A which has, e.g., at most one, two, three, four, five, six, seven, eight, nine, or ten amino acid substitutions relative to the FGFR3 of SEQ ID NO: 13.

In other aspects of this embodiment, the FGFR3 can be a human FGFR3111S that selectively binds BoNT/A which has, e.g., at least 70% amino acid identity with the FGFR3 of SEQ ID NO: 15, at least 75% amino acid identity with the FGFR3 of SEQ ID NO: 15, at least 80% amino acid identity with the FGFR3 of SEQ ID NO: 15, at least 85% amino acid identity with the FGFR3 of SEQ ID NO: 15, at least 90% amino acid identity with the FGFR3 of SEQ ID NO: 15 or at least 95% amino acid identity with the FGFR3 of SEQ ID NO: 15. In other aspects of this embodiment, the FGFR3 is a human FGFR3111S that that selectively binds BoNT/A which has, e.g., at most one, two, three, four, five, six, seven, eight, nine, or ten amino acid substitutions relative to the FGFR3 of SEQ ID NO: 15.

In other aspects of this embodiment, the FGFR3 can be a bovine FGFR3IIIc that selectively binds BoNT/A which has, e.g., at least 70% amino acid identity with the FGFR3 of SEQ ID NO: 17, at least 75% amino acid identity with the FGFR3 of SEQ ID NO: 17, at least 80% amino acid identity with the FGFR3 of SEQ ID NO: 17, at least 85% amino acid identity with the FGFR3 of SEQ ID NO: 17, at least 90% amino acid identity with the FGFR3 of SEQ ID NO: 17 or at least 95% amino acid identity with the FGFR3 of SEQ ID NO: 17. In other aspects of this embodiment, the FGFR3 is a bovine FGFR3IIIc that that selectively binds BoNT/A which has, e.g., at most one, two, three, four, five, six, seven, eight, nine, or ten amino acid substitutions relative to the FGFR3 of SEQ ID NO: 17.

In other aspects of this embodiment, the FGFR3 can be a mouse FGFR3IIIb that selectively binds BoNT/A which has, e.g., at least 70% amino acid identity with the FGFR3 of SEQ ID NO: 19, at least 75% amino acid identity with the FGFR3 of SEQ ID NO: 19, at least 80% amino acid identity with the FGFR3 of SEQ ID NO: 19, at least 85% amino acid identity with the FGFR3 of SEQ ID NO: 19, at least 90% amino acid identity with the FGFR3 of SEQ ID NO: 19 or at least 95% amino acid identity with the FGFR3 of SEQ ID NO: 19. In other aspects of this embodiment, the FGFR3 is a mouse FGFR3IIIc that that selectively binds BoNT/A which has, e.g., at most one, two, three, four, five, six, seven, eight, nine, or ten amino acid substitutions relative to the FGFR3 of SEQ ID NO: 19.

In other aspects of this embodiment, the FGFR3 can be a mouse FGFR3IIIc that selectively binds BoNT/A which has, e.g., at least 70% amino acid identity with the FGFR3 of SEQ ID NO: 21, at least 75% amino acid identity with the FGFR3 of SEQ ID NO: 21, at least 80% amino acid identity with the FGFR3 of SEQ ID NO: 21, at least 85% amino acid identity with the FGFR3 of SEQ ID NO: 21, at least 90% amino acid identity with the FGFR3 of SEQ ID NO: 21 or at least 95% amino acid identity with the FGFR3 of SEQ ID NO: 21. In other aspects of this embodiment, the FGFR3 is a mouse FGFR3IIIc that that selectively binds BoNT/A which has, e.g., at most one, two, three, four, five, six, seven, eight, nine, or ten amino acid substitutions relative to the FGFR3 of SEQ ID NO: 21.

In other aspects of this embodiment, the FGFR3 can be a mouse FGFR3-delAcid that selectively binds BoNT/A which has, e.g., at least 70% amino acid identity with the FGFR3 of SEQ ID NO: 23, at least 75% amino acid identity with the FGFR3 of SEQ ID NO: 23, at least 80% amino acid identity with the FGFR3 of SEQ ID NO: 23, at least 85% amino acid identity with the FGFR3 of SEQ ID NO: 23, at least 90% amino acid identity with the FGFR3 of SEQ ID NO: 23 or at least 95% amino acid identity with the FGFR3 of SEQ ID NO: 23. In other aspects of this embodiment, the FGFR3 is a mouse FGFR3-delAcid that that selectively binds BoNT/A which has, e.g., at most one, two, three, four, five, six, seven, eight, nine, or ten amino acid substitutions relative to the FGFR3 of SEQ ID NO: 23.

In other aspects of this embodiment, the FGFR3 can be a rat FGFR3IIIb that selectively binds BoNT/A which has, e.g., at least 70% amino acid identity with the FGFR3 of SEQ ID NO: 25, at least 75% amino acid identity with the FGFR3 of SEQ ID NO: 25, at least 80% amino acid identity with the FGFR3 of SEQ ID NO: 25, at least 85% amino acid identity with the FGFR3 of SEQ ID NO: 25, at least 90% amino acid identity with the FGFR3 of SEQ ID NO: 25 or at least 95% amino acid identity with the FGFR3 of SEQ ID NO: 25. In other aspects of this embodiment, the FGFR3 is a rat FGFR3IIIb that that selectively binds BoNT/A which has, e.g., at most one, two, three, four, five, six, seven, eight, nine, or ten amino acid substitutions relative to the FGFR3 of SEQ ID NO: 25.

In other aspects of this embodiment, the FGFR3 can be a rat FGFR3IIIc that selectively binds BoNT/A which has, e.g., at least 70% amino acid identify with the FGFR3 of SEQ ID NO: 27, at least 75% amino acid identity with the FGFR3 of SEQ ID NO: 27, at least 80% amino acid identity with the FGFR3 of SEQ ID NO: 27, at least 85% amino acid identity with the FGFR3 of SEQ ID NO: 27, at least 90% amino acid identity with the FGFR3 of SEQ ID NO: 27 or at least 95% amino acid identity with the FGFR3 of SEQ ID NO: 27. In other aspects of this embodiment, the FGFR3 is a rat FGFR3IIIc that that selectively binds BoNT/A which has, e.g., at most one, two, three, four, five, six, seven, eight, nine, or ten amino acid substitutions relative to the FGFR3 of SEQ ID NO: 27.

In other aspects of this embodiment, the FGFR3 can be a chicken FGFR3 that selectively binds BoNT/A which has, e.g., at least 70% amino acid identity with the FGFR3 of SEQ ID NO: 29, at least 75% amino acid identity with the FGFR3 of SEQ ID NO: 29, at least 80% amino acid identity with the FGFR3 of SEQ ID NO: 29, at least 85% amino acid identity with the FGFR3 of SEQ ID NO: 29, at least 90% amino acid identity with the FGFR3 of SEQ ID NO: 29 or at least 95% amino acid identity with the FGFR3 of SEQ ID NO: 29. In other aspects of this embodiment, the FGFR3 is a chicken FGFR3 that that selectively binds BoNT/A which has, e.g., at most one, two, three, four, five, six, seven, eight, nine, or ten amino acid substitutions relative to the FGFR3 of SEQ ID NO: 29.

In other aspects of this embodiment, the FGFR3 can be a frog FGFR3-1 that selectively binds BoNT/A which has, e.g., at least 70% amino acid identity with the FGFR3 of SEQ ID NO: 31, at least 75% amino acid identity with the FGFR3 of SEQ ID NO: 31, at least 80% amino acid identity with the FGFR3 of SEQ ID NO: 31, at least 85% amino acid identity with the FGFR3 of SEQ ID NO: 31, at least 90% amino acid identity with the FGFR3 of SEQ ID NO: 31 or at least 95% amino acid identity with the FGFR3 of SEQ ID NO: 31. In other aspects of this embodiment, the FGFR3 is a frog FGFR3 that that selectively binds BoNT/A which has, e.g., at most one, two, three, four, five, six, seven, eight, nine, or ten amino acid substitutions relative to the FGFR3 of SEQ ID NO: 31.

In other aspects of this embodiment, the FGFR3 can be a frog FGFR3-2 that selectively binds BoNT/A which has, e.g., at least 70% amino acid identity with the FGFR3 of SEQ ID NO: 33, at least 75% amino acid identity with the FGFR3 of SEQ ID NO: 33, at least 80% amino acid identity with the FGFR3 of SEQ ID NO: 33, at least 85% amino acid identity with the FGFR3 of SEQ ID NO: 33, at least 90% amino acid identity with the FGFR3 of SEQ ID NO: 33 or at least 95% amino acid identity with the FGFR3 of SEQ ID NO: 33. In other aspects of this embodiment, the FGFR3 is a frog FGFR3 that that selectively binds BoNT/A which has, e.g., at most one, two, three, four, five, six, seven, eight, nine, or ten amino acid substitutions relative to the FGFR3 of SEQ ID NO: 33.

In other aspects of this embodiment, the FGFR3 can be a newt FGFR3 that selectively binds BoNT/A which has, e.g., at least 70% amino acid identity with the FGFR3 of SEQ ID NO: 35, at least 75% amino acid identity with the FGFR3 of SEQ ID NO: 35, at least 80% amino acid identity with the FGFR3 of SEQ ID NO: 35, at least 85% amino acid identity with the FGFR3 of SEQ ID NO: 35, at least 90% amino acid identity with the FGFR3 of SEQ ID NO: 35 or at least 95% amino acid identity with the FGFR3 of SEQ ID NO: 35. In other aspects of this embodiment, the FGFR3 is a newt FGFR3 that that selectively binds BoNT/A which has, e.g., at most one, two, three, four, five, six, seven, eight, nine, or ten amino acid substitutions relative to the FGFR3 of SEQ ID NO: 35.

In other aspects of this embodiment, the FGFR3 can be a zebrafish FGFR3 that selectively binds BoNT/A which has, e.g., at least 70% amino acid identity with the FGFR3 of SEQ ID NO: 37, at least 75% amino acid identity with the FGFR3 of SEQ ID NO: 37, at least 80% amino acid identity with the FGFR3 of SEQ ID NO: 37, at least 85% amino acid identity with the FGFR3 of SEQ ID NO: 37, at least 90% amino acid identity with the FGFR3 of SEQ ID NO: 37 or at least 95% amino acid identity with the FGFR3 of SEQ ID NO: 37. In other aspects of this embodiment, the FGFR3 is a zebrafish FGFR3 that that selectively binds BoNT/A which has, e.g., at most one, two, three, four, five, six, seven, eight, nine, or ten amino acid substitutions relative to the FGFR3 of SEQ ID NO: 37.

Non-limiting examples of specific methods that can be used to make an use BoNT/A receptors disclosed in the present specification are described in, see e.g., Molecular Cloning, A Laboratory Manual, supra, (2001) and Current Protocols in Molecular Biology, supra, (2004), which are hereby incorporated by reference. In addition, non-limiting examples of cloning and expression kits, as well as well-characterized reagents, conditions and protocols are readily available from commercial vendors that include, without limitation, BD Biosciences-Clontech, Palo Alto, Calif.; Invitrogen, Inc, Carlsbad, Calif.; and Stratagene, La Jolla, Calif. These protocols are routine procedures well within the scope of one skilled in the art and from the teaching herein.

It is envisioned that any and all compositions comprising a BoNT/A receptor capable of binding a BoNT/A toxin disclosed in the present specification, but not capable of binding a BoNT/A toxin bound to a neutralizing anti-BoNT/A antibody, can be used in methods disclosed in the present specification. Non-limiting examples of compositions comprising a BoNT/A receptor include, e.g., synaptosomes, synaptic vesicles, synaptic membranes, synaptic densities, and the like, obtained from tissues; and synaptosomes, synaptic vesicles, synaptic membranes, synaptic densities, and the like, obtained from tissues cell lines. Tissue enriched for nerve cells capable of being intoxicated by a BoNT/A toxin include, without limitation, brain, spinal cord, retina, sympathetic ganglia, myenteric plexus and electric organs. Neuronal cells useful in aspects of the invention include, without limitation, primary neuronal cells; immortalized or established neuronal cells; transformed neuronal cells; neuronal tumor cells; stably and transiently transfected neuronal cells and further include, yet are not limited to, mammalian, murine, rat, primate and human neuronal cells. Non-limiting examples of neuronal cells useful in aspects of the invention include, e.g., peripheral neuronal cells, such as, e.g., motor neurons and sensory neurons; and CNS neuronal cells, such as, e.g., spinal cord neurons like embryonic spinal cord neurons, dorsal root ganglia (DRG) neurons, cerebral cortex neurons, cerebellar neurons, hippocampal neurons and motor neurons. Neuronal cells useful in the invention can be, for example, central nervous system (CNS) neurons; neuroblastoma cells; motor neurons, hippocampal neurons or cerebellar neurons and further can be, without limitation, Neuro-2A, SH-SY5Y, NG108-15, N1E-115 or SK-N-DZ cells. The skilled person understands that these and additional primary and established neurons can be useful in the cells and methods of the invention.

Thus, in an embodiment, a preparation derived from a tissue capable of being intoxicated by a BoNT/A toxin can be the source of receptor. In another embodiment, a preparation derived from a cell line capable of being intoxicated by a BoNT/A toxin can be the source of BoNT/A receptor, such as, e.g., primary cell lines originating from brain, spinal cord, retina, sympathetic ganglia, myenteric plexus and electric organs; and established cell lines, such as, e.g., SH-SY5Y, SK-N-DZ, SK-N-F1, SK-N-SH, BE (2)-C, Neuro-2A, NB4 1A3, NIE-115, NG108-15 and PC12. Non-limiting examples of tissue preparations containing receptors include, e.g., synaptosomes, synaptic vesicles, synaptic membranes, synaptic densities, and the like. Furthermore, the receptor-containing tissue preparation can be from a variety of organisms including, but not limited to, mammals, such as, e.g. rodents, rabbits, porcines, bovines, equines, non-human primates and humans. Thus aspects of this embodiment may include, e.g., mammalian synaptosome preparations; mammalian synaptic vesicle preparations; mammalian synaptic membrane preparations and mammalian synaptic density preparations. In other aspect of this embodiment, a mouse synaptosome preparation is used, a rat synaptosome preparation is used, a mouse synaptic vesicle preparation is used, a rat synaptic vesicle preparation is used. Non-limiting examples of specific protocols for making and using synaptosome, synaptic vesicle, synaptic membrane and synaptic density preparations are described in, e.g., Victor P. Whittaker, The Synaptosomes, 1-39 (HANDBOOK OF NEUROCHEMISTRY, Vol 7, Abel Lathia ed., Plenum Press, 1984); Victor P. Whittaker, Thirty Years of Synaptosome Research, 22(9) J. NEUROCYTOL. 735-42, (1993); NEUROCHEMISTRY: A PRACTICAL APPROACH (Anthony J. Turner & Herman S. Bachelard eds., Oxford University Press, 2^{nd} ed. 1997) which are hereby incorporated by reference. These protocols are routine procedures well within the scope of one skilled in the art and from the teaching herein.

Aspects of the present invention provide, in part, detecting the amount of toxin-receptor complexes formed. In one embodiment, the presence of toxin-receptor complexes is determined by detecting the amount of toxin-receptor complexes formed, where the presence of the toxin-receptor complexes indicates the lack of BoNT/A immunoresistance. In another embodiment, the absence of toxin-receptor complexes is determined by detecting the amount of toxin-receptor complexes formed, where the absence of the toxin-receptor complexes indicates BoNT/A immunoresistance.

Aspects of the present invention provide, in part, detecting the amount of free BoNT/A receptor. As used herein, the term “free BoNT/A receptor” means any BoNT/A receptor not bound by a BoNT/A toxin.

In one embodiment, the presence of BoNT/A receptor is determined by detecting the amount of free receptor not bound in toxin-receptor complexes, where the presence of free BoNT/A receptor indicates the BoNT/A immunoresistance. In another embodiment, the absence of free BoNT/A receptor complexes is determined by detecting the amount of free receptor not bound in toxin-receptor complexes, where the absence of free BoNT/A receptor indicates a lack of BoNT/A immunoresistance.

Aspects of the present invention provide, in part, detecting the amount of toxin-antibody complexes formed. In one embodiment, the presence of toxin-antibody complexes is determined by detecting the amount of toxin-antibody complexes formed, where the presence of the toxin-antibody complexes indicates BoNT/A immunoresistance. In another embodiment, the absence of toxin-antibody complexes is determined by detecting the amount of toxin-antibody complexes formed, where the absence of the toxin-antibody complexes indicates the lack of BoNT/A immunoresistance.

In is envisioned that any and all detection methods suitable for indicating the presence or absence of BoNT/A in a toxin-receptor complex, a free BoNT/A receptor or a toxin-antibody complex can be employed including without limitation, a radiation detection method, a fluorescence detection method, a fluorescence resonance energy transfer (FRET) detection method, a phosphorescence detection method. a chemiluminescence detection method, a bioluminescence detection method, an electrochemiluminescence detection method, a chromagenic detection method and an enzyme-activity detection method. Thus, aspect of this embodiment may include a detection method that indicates the presence or absence of a BoNT/A toxin, a detection method that indicates the presence or absence of a BoNT/A receptor from a tissue preparations containing BoNT/A receptors include, e.g., synaptosomes, synaptic vesicles, synaptic membranes, synaptic densities, and the like or a detection method that indicates the presence or absence of a BoNT/A receptor protein, such as, e.g., FGFR3 and members of the synaptotagmin family. Other aspects of this embodiment may include a detection method that indicates the presence or absence of a toxin-receptor complex or a detection method that indicates the presence or absence of a toxin-antibody complex.

It is further foreseen that a detection method can either qualitatively or quantitatively determine the presence of a toxin-receptor complex or free BoNT/A receptor. Qualitative measurements can be determined by a wide variety of methods, such as, e.g., audioradiography, immunoblotting techniques, and the like. Quantitative measurements can be determined by a wide variety of methods, such as, e.g., scintillation counters, spectrophotometers, densitometers, fluorometers, spectroluminometers, luminometers, high pressure liquid chromatography, and the like. In addition, control samples can also be assayed with a test sample using this method in order to provide baseline values useful for comparisons with a test sample. Thus, a negative control comprises a sample known not to contain any neutralizing anti-BoNT/A antibodies. A negative control can establish a parameter for background noise levels and provide a means to distinguish false positive results from an actual BoNT/A immune resistance response. A sample known to contain high levels of neutralizing anti-BoNT/A antibodies from an individual diagnosed with BoNT/A immunoresistance could serve as a positive control. A positive control can provide a parameter from which a test sample can be evaluated to determine the relative severity of immunoresistance occurring in a test patient. One skilled in the art understands that, if desired, a quantitative method can be used for qualitative measurements. In addition, one skilled in the art understands that the selection of a method of measurement is determined by the detection means employed.

In addition, any of a variety of marker compounds suitable for the detection system selected, can be operably-linked to a BoNT/A toxin or BoNT/A receptor as a labeled molecule including, without limitation, a radioisotope, fluorescent compound, a phosphorescent compound, a chemiluminescent compound, a bioluminescent compound, and the like. Thus, in one aspect of the present invention, a marker compound suitable for the detection system selected, is operably-linked to a BoNT/A toxin as the labeled molecule suitable for any method. In another aspect of the present invention, a marker compound suitable for the detection system selected, is operably-linked to a receptor, such as, e.g., a synaptosome preparation or a receptor protein, as the labeled molecule suitable for any method. The term “operably linked” as used herein, in reference to a labeled molecule, means any of a variety of chemical reactions that can join a marker compound disclosed in the present specification to a molecule disclosed in the present specification such that a single peptide, comprising a peptide and marker compound, suitable to perform a method disclosed in the present specification is produced.

Non-limiting examples of radioisotopes that may be operably-linked to a BoNT/A toxin or BoNT/A receptor disclosed in the specification include, e.g., ³Hydrogen, ¹⁴Carbon, ²²Sodium, ³²Phosphorus, ³³Phosphorus, ³⁵Sulfer, ³⁶Chlorine, ⁴⁵Calcium, ⁵¹Chromium, ⁵⁷Cobalt, ⁵⁸Cobalt, ⁵⁹Iron, ⁶³Nickel, ⁶⁵Zinc, ⁷⁵Selenium, ⁸⁶Rubidum, ¹⁰³Ruthenium, ¹⁰⁹Cadmium, ¹²⁵Iodine, ¹³¹Iodine, and the like. Non-limiting examples of fluorescent compounds that may be operably-linked to a BoNT/A toxin or BoNT/A receptor disclosed in the specification include, e.g., fluorescein, fluorescamine, isocyanate, isothiocyanate, rhodamine, phycoerythrin, phycocyanin, allophycocyanin, o-phthaldehyde, Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Cy-2, Cy-3, Cy-5, Cy-7 and the like. Non-limiting examples of chemiluminescent compounds that may be operably-linked to a BoNT/A toxin or BoNT/A receptor disclosed in the specification include, e.g., imidazoles, such as, e.g., lophine; acylhydrazines, such as, e.g., luminal and isoluminol; acridinium salts and esters, such as, e.g., lucigenin; oxalate salts and esters, such as, e.g., bis(2,4,6-trichloropheryl)oxalate (TCPO) and bis(2,4,-dinitrophenyl) oxalate (DNPO); Tris (2,2N-bipyridine) ruthenium compounds, such as, e.g., ruthenium(bipyridine)₃, and the like. Non-limiting examples of bioluminescent compounds that may be operably-linked to a BoNT/A toxin or BoNT/A receptor disclosed in the specification include, e.g., bacterial luciferins, dinoflagellate luciferins, vargulins, porichthys luciferins, coelenterazines, beetle luciferins, 4-methylumbelliferone esters, and the like.

Likewise, any of a variety of peptides suitable for the detection method selected, can be operably-linked to a BoNT/A toxin or BoNT/A receptor as a fusion protein including, without limitation, a peptide necessary for producing florescence, a peptide necessary for producing phosphorescence, a peptide necessary for producing chemiluminescence, a peptide necessary for producing bioluminescence, and the like. The term “operably linked” as used herein, in reference to a fusion protein, means any of a variety of cloning methods that can join a first nucleic acid sequence composition encoding a first peptide disclosed in the present specification in-frame with a second nucleic acid sequence composition encoding a second peptide disclosed in the present specification such that a single peptide, comprising both the first and second peptides, suitable to perform a method disclosed in the present specification is produced when expressed. In one embodiment, a peptide suitable for the detection method selected, is operably-linked to a BoNT/A toxin. In another embodiment, a peptide suitable for the detection method selected, is operably-linked to a BoNT/A receptor.

Non-limiting examples of a peptide necessary for producing florescence that may be operably-linked to a BoNT/A toxin or BoNT/A receptor disclosed in the specification include, e.g., photoproteins, such as, e.g., aequorin; obelin; Aequorea fluorescent proteins, such, e.g., green fluorescent protein (GFP), cyan fluorescent protein (CFP), blue fluorescent protein (BFP), red fluorescent protein (RFP), yellow fluorescent protein (YFP), ultraviolet fluorescent protein (GFPuv), their fluorescence-enhancement variants, including EGFP, ECFP, EBFP and EYFP, their peptide destabilization variants, and the like; and Red coral reef fluorescent proteins (RCFPs), such, e.g., Discosoma red fluorescent protein (DsRed), Anemonia red fluorescent protein (AsRed), Heteractis far-red fluorescent protein (HcRed), Anemonia cyan fluorescent protein (AmCyan), Zoanthus green fluorescent protein (ZsGreen), Zoanthus yellow fluorescent protein (ZsYellow), their fluorescence-enhancement variants, including DsRed2, AsRed2, their peptide destabilization variants, and the like. Non-limiting examples of a peptide necessary for producing chemiluminescence that may be operably-linked to a BoNT/A toxin or BoNT/A receptor disclosed in the specification include, e.g., alkaline phosphatases, horseradish peroxidases, xanthine oxidases, glucose oxidases and β-galatosidases. Non-limiting examples of a peptide necessary for producing bioluminescence that may be operably-linked to a BoNT/A toxin or BoNT/A receptor disclosed in the specification include, e.g., bacterial luciferases, dinoflagellate luciferases, vargula luciferases, coelenterate luciferases, beetle luciferases, and the like. Non-limiting examples of a peptide necessary for producing chromogenic compound that may be operably-linked to a BoNT/A toxin or BoNT/A receptor disclosed in the specification include, e.g., alkaline phosphatases, horseradish peroxidases, ureases, β-glucourinidases, glucose oxidases and β-galatosidases.

Non-limiting examples of specific protocols for selecting, making and using detection systems, making and using peptides labeled with a marker compound and making and using fusion proteins are described in, e.g., MOLECULAR CLONING A LABORATORY MANUAL, supra, (2001); METHODS IN ENZYMOLOGY, VOL. 305, BIOLUMINESCENCE AND CHEMILUMINESCENCE, PART C (Miriam M. Ziegler & Thomas O. Baldwin eds., Academic Press, 2000); Y. Fuster Mestre et al., Flow-chemiluminescence: A Growing Modality of Pharmaceutocal Analysis, 16 LUMINESCENCE 213-235, (2001); Lee F. Greer III & Aladar A. Szalay, Imaging of Light Emission From the Expression of Luciferases in Living Cells and Organisms: A Review, 17 LUMINESCENCE 43-74, (2002); Richard W. Horobin & John A. Kiernan, CONN's BIOLOGICAL STAINS: A HANDBOOK OF DYES, STAINS AND FLUOROCHROMES FOR USE IN BIOLOGY AND MEDICINE (BIOS Scientific Publishers, 10^{th} ed. 2002); HANDBOOK OF FLUORESCENT PROBES AND RESEARCH CHEMICALS, http:flwww.probes.com/handbook (Molecular Probes, Inc., 9^{th} ed, 2004), and CURRENT PROTOCOLS IN MOLECULAR BIOLOGY, supra, (2004), which are hereby incorporated by reference. In addition, non-limiting examples of how to make and use detection systems, labeled peptides and fusion protein disclosed in the present specification, as well as well-characterized reagents, conditions and protocols are readily available from commercial vendors that include, without limitation, Amersham Biosciences, Piscataway, N.J.; BD Biosciences-Clontech, Palo Alto, Calif.; Bio-Rad Laboratories, Hercules, Calif.; Cayman Chemical Co., Ann Arbor, Mich.; Molecular Probes, Inc., Eugene, Oreg.; PerkinElmer Life and Analytical Sciences, Inc., Boston, Mass.; Pierce Biotechnology, Inc., Rockford, Ill.; Princeton Separations, Adelphia, N.J.; and Vector Laboratories, Burlingame, Calif. These protocols are routine procedures within the scope of one skilled in the art and from the teaching herein.

Thus, in an embodiment, a BoNT/A toxin is operably-linked to a radioisotope. In an aspect of this embodiment, a BoNT/A toxin is operably-linked to ³Hydrogen, ¹⁴Carbon, ²²Sodium, ³²Phosphorus, ³³Phosphorus, ³⁵Sulfer, ³⁶Chlorine, ⁴⁵Calcium, ⁵¹Chromium, ⁵⁷Cobalt, ⁵⁸Cobalt, ⁵⁹Iron, ⁶³Nickel, ⁶⁵Zinc, ⁷⁵Selenium, ⁸⁶Rubidum, ¹⁰³Ruthenium, ¹⁰⁹Cadmium, ¹²⁵Iodine or ¹³¹Iodine. In another aspect of this embodiment, the presence of BoNT/A toxin-BoNT/A receptor complexes is quantitatively determined using a scintillation counter. In another aspect of this embodiment, the absence of BoNT/A toxin-BoNT/A receptor complexes is quantitatively determined using a scintillation counter. In another embodiment, a BoNT/A receptor is operably-linked to a radioisotope. In an aspect of this embodiment, a BoNT/A receptor is operably-linked to ³Hydrogen, ¹⁴Carbon, ²²Sodium, ³²Phosphorus, ³³Phosphorus, ³⁵Sulfer, ³⁶Chlorine, ⁴⁵Calcium, ⁵¹Chromium, ⁵⁷Cobalt, ⁵⁸Cobalt, ⁵⁹Iron, ⁶³Nickel, ⁶⁵Zinc, ⁷⁵Selenium, ⁸⁶Rubidum, ¹⁰³Ruthenium, ¹⁰⁹Cadmium, ¹²⁵Iodine or ¹³¹Iodine. In another aspect of this embodiment, a FGFR3 is operably-linked to a radioisotope. In another aspect of this embodiment, a FGFR3 is operably-linked to 3Hydrogen, ¹⁴Carbon, 22Sodium, ³²Phosphorus, ³³Phosphorus, ³⁵Sulfer, ³⁶Chlorine, ⁴⁵Calcium, ⁵¹Chromium, ⁵⁷Cobalt, ⁵³Cobalt, ⁵⁹Iron, ⁶³Nickel, 65Zinc, ⁷⁵Selenium, ⁸⁶Rubidum, ¹⁰³Ruthenium, ¹⁰⁹Cadmium, ¹²⁵Iodineor ¹³¹Iodine. In another aspect of this embodiment, the presence of BoNT/A toxin-BoNT/A receptor complexes is quantitatively determined using a scintillation counter. In another aspect of this embodiment, the absence of BoNT/A toxin-BoNT/A receptor complexes is quantitatively determined using a scintillation counter. In another aspect of this embodiment, the presence of BoNT/A toxin-FGFR3 complexes is quantitatively determined using a scintillation counter (see, e.g., Example 13).

In another aspect of this embodiment, the absence of BoNT/A toxin-FGFR3 complexes is quantitatively determined using a scintillation counter.

In yet another embodiment, a BoNT/A toxin is operably-linked to a fluorescent compound. In an aspect of this embodiment, a BoNT/A toxin is operably-linked to a fluorescein, a fluorescamine, an isocyanate, an isothiocyanate, a rhodamine, a phycoerythrin, a phycocyanin, an allophycocyanin, an o-phthaldehyde, an Alexa Fluor® 350, an Alexa Fluor® 430, an Alexa Fluor® 488, an Alexa Fluor® 532, an Alexa Fluor® 546, an Alexa Fluor® 555, an Alexa Fluor® 568, an Alexa Fluor® 594, an Alexa Fluor® 633, an Alexa Fluor® 647, an Alexa Fluor® 660, an Alexa Fluor® 680, an Alexa Fluor® 700, an Alexa Fluor® 750, a Cy-2, a Cy-3, a Cy-5 or a Cy-7. In another aspect of this embodiment, the presence of BoNT/A toxin-BoNT/A receptor complexes is quantitatively determined using a spectrofluorometer (see, e.g., Example 7). In another aspect of this embodiment, the absence of BoNT/A toxin-BoNT/A receptor complexes is quantitatively determined using a spectrofluorometer. In another embodiment, a BoNT/A receptor is operably-linked to a fluorescent compound. In an aspect of this embodiment, a BoNT/A receptor is operably-linked to a fluorescent compound. In another aspect of this embodiment, a BoNT/A receptor is operably-linked to a fluorescein, a fluorescamine, an isocyanate, an isothiocyanate, a rhodamine, a phycoerythrin, a phycocyanin, an allophycocyanin, an o-phthaldehyde, an Alexa Fluor® 350, an Alexa Fluor® 430, an Alexa Fluor® 488, an Alexa Fluor® 532, an Alexa Fluor® 546, an Alexa Fluor® 555, an Alexa Fluor® 568, an Alexa Fluor® 594, an Alexa Fluor® 633, an Alexa Fluor® 647, an Alexa Fluor® 660, an Alexa Fluor® 680, an Alexa Fluor® 700, an Alexa Fluor® 750, a Cy-2, a Cy-3, a Cy-5 or a Cy-7. In an aspect of this embodiment, a FGFR3 is operably-linked to a fluorescent compound. In another aspect of this embodiment, a FGFR3 is operably-linked to a fluorescein, a fluorescamine, an isocyanate, an isothiocyanate, a rhodamine, a phycoerythrin, a phycocyanin, an allophycocyanin, an o-phthaldehyde, an Alexa Fluor® 350, an Alexa Fluor® 430, an Alexa Fluor® 488, an Alexa Fluor® 532, an Alexa Fluor® 546, an Alexa Fluor® 555, an Alexa Fluor® 568, an Alexa Fluor® 594, an Alexa Fluor® 633, an Alexa Fluor® 647, an Alexa Fluor® 660, an Alexa Fluor® 680, an Alexa Fluor® 700, an Alexa Fluor® 750, a Cy-2, a Cy-3, a Cy-5 or a Cy-7. In still another aspect of this embodiment, the presence of BoNT/A toxin-BoNT/A receptor complexes is quantitatively determined using a spectrofluorometer. In still another aspect of this embodiment, the absence of BoNT/A toxin-BoNT/A receptor complexes is quantitatively determined using a spectrofluorometer. In yet another aspect of this embodiment, the presence of BoNT/A toxin-FGFR3 complexes is quantitatively determined using a spectrofluorometer. In yet another aspect of this embodiment, the absence of BoNT/A toxin-FGFR3 complexes is quantitatively determined using a spectrofluorometer.

In another embodiment, a BoNT/A toxin is operably-linked to a photoprotein. In an aspect of this embodiment, a BoNT/A toxin is operably linked to an aequorin, an obelin, a GFP, an EGFP, a CFP, an ECFP, a BFP, an EBFP, a YFP, an EYFP, a GFPuv, a DsRed, a DsRed2, a AsRed, a AsRed2, a H_{C}Red, an AmCyan, a ZsGreen or a ZsYellow. In another aspect of this embodiment, the presence of BoNT/A toxin-BoNT/A receptor complexes is quantitatively determined using a spectrofluorometer. In another aspect of this embodiment, the absence of BoNT/A toxin-BoNT/A receptor complexes is quantitatively determined using a spectrofluorometer. In another embodiment, a BoNT/A receptor is operably-linked to a photoprotein. In an aspect of this embodiment, a BoNT/A receptor is operably-linked to an aequorin, an obelin, a GFP, an EGFP, a CFP, an ECFP, a BFP, an EBFP, a YFP, an EYFP, a GFPuv, a DsRed, a DsRed2, a AsRed, a AsRed2, a H_{C}Red, an AmCyan, a ZsGreen or a ZsYellow. In an aspect of this embodiment, a FGFR3 is operably-linked to an aequorin, an obelin, a GFP, an EGFP, a CFP, an ECFP, a BFP, an EBFP, a YFP, an EYFP, a GFPuv, a DsRed, a DsRed2, a AsRed, a AsRed2, a H_{C}Red, an AmCyan, a ZsGreen or a ZsYellow. In still another aspect of this embodiment, the presence of BoNT/A toxin-BoNT/A receptor complexes is quantitatively determined using a spectrofluorometer. In still another aspect of this embodiment, the absence of BoNT/A toxin-BoNT/A receptor complexes is quantitatively determined using a spectrofluorometer. In yet another aspect of this embodiment, the presence of BoNT/A toxin-FGFR3 complexes is quantitatively determined using a spectrofluorometer. In yet another aspect of this embodiment, the absence of BoNT/A toxin-FGFR3 complexes is quantitatively determined using a spectrofluorometer.

In yet another embodiment, a BoNT/A toxin is operably-linked to a chemiluminescent compound. In an aspect of this embodiment, a BoNT/A toxin is operably linked to an imidazole, an acridinium salt, an acridinium ester, an oxalate salt, an oxalate ester, or a Tris (2,2N-bipyridine) ruthenium compound. In another aspect of this embodiment, the presence of BoNT/A toxin-BoNT/A receptor complexes is quantitatively determined using a luminometer. In another aspect of this embodiment, the absence of BoNT/A toxin-BoNT/A receptor complexes is quantitatively determined using a luminometer. In another embodiment, a BONT/A receptor is operably-linked to a chemiluminescent compound. In an aspect of this embodiment, a BoNT/A receptor is operably-linked to an imidazole, an acridinium salt, an acridinium ester, an oxalate salt, an oxalate ester, or a Tris (2,2N-bipyridine) ruthenium compound. In an aspect of this embodiment, a FGFR3 is operably-linked to a chemiluminescent compound. In another aspect of this embodiment, a FGFR3 is operably-linked to an imidazole, an acridinium salt, an acridinium ester, an oxalate salt, an oxalate ester, or a Tris (2,2N-bipyridine) ruthenium compound. In still another aspect of this embodiment, the presence of BoNT/A toxin-BoNT/A receptor complexes is quantitatively determined using a luminometer. In still another aspect of this embodiment, the absence of BoNT/A toxin-BoNT/A receptor complexes is quantitatively determined using a luminometer. In yet another aspect of this embodiment, the presence of BoNT/A toxin-FGFR3 complexes is quantitatively determined using a luminometer. In yet another aspect of this embodiment, the absence of BoNT/A toxin-FGFR3 complexes is quantitatively determined using a luminometer.

In yet another embodiment, a BoNT/A toxin is operably-linked to a peptide necessary for producing chemiluminescence. In an aspect of this embodiment, a BoNT/A toxin is operably linked to an alkaline phosphatase, a horseradish peroxidase, a xanthine oxidase, a glucose oxidase or a β-galatosidase. In another aspect of this embodiment, the presence of BoNT/A toxin-receptor complexes is quantitatively determined using a luminometer. In another aspect of this embodiment, the absence of BoNT/A toxin-receptor complexes is quantitatively determined using a luminometer. In another embodiment, a BoNT/A receptor is operably-linked to a peptide necessary for producing chemiluminescence. In an aspect of this embodiment, a BoNT/A receptor is operably linked to an alkaline phosphatase, a horseradish peroxidase, a xanthine oxidase, a glucose oxidase or a β-galatosidase. In another aspect of this embodiment, a FGFR3 is operably linked to a peptide necessary for producing chemiluminescence. In another aspect of this embodiment, a FGFR3 is operably linked to an alkaline phosphatase, a horseradish peroxidase, a xanthine oxidase, a glucose oxidase or a β-galatosidase. In still another aspect of this embodiment, the presence of BoNT/A toxin-BoNT/A receptor complexes is quantitatively determined using a luminometer. In still another aspect of this embodiment, the absence of BoNT/A toxin-BoNT/A receptor complexes is quantitatively determined using a luminometer. In yet another aspect of this embodiment, the presence of BoNT/A toxin-FGFR3 complexes is quantitatively determined using a luminometer. In yet another aspect of this embodiment, the absence of BoNT/A toxin-FGFR3 complexes is quantitatively determined using a luminometer.

In yet another embodiment, a BoNT/A toxin is operably-linked to a peptide necessary for producing bioluminescence. In an aspect of this embodiment, a BoNT/A toxin is operably linked to a bacterial luciferase, a dinoflagellate luciferase, a vargula luciferase, a coelenterate luciferase or a beetle luciferase. In another aspect of this embodiment, the presence of BoNT/A toxin-BoNT/A receptor complexes is quantitatively determined using a luminometer (see, e.g., Example 12). In another aspect of this embodiment, the absence of BoNT/A toxin-BoNT/A receptor complexes is quantitatively determined using a luminometer. In another embodiment, a BoNT/A receptor is operably-linked to a peptide necessary for producing bioluminescence. In an aspect of this embodiment, a BoNT/A receptor is operably linked to a bacterial luciferase, a dinoflagellate luciferase, a vargula luciferase, a coelenterate luciferase or a beetle luciferase. In another aspect of this embodiment, a FGFR3 is operably linked to a peptide necessary for producing bioluminescence. In an aspect of this embodiment, a FGFR3 is operably linked to a bacterial luciferase, a dinoflagellate luciferase, a vargula luciferase, a coelenterate luciferase or a beetle luciferase. In still another aspect of this embodiment, the presence of BoNT/A toxin-BoNT/A receptor complexes is quantitatively determined using a luminometer. In still another aspect of this embodiment, the absence of BoNT/A toxin-BoNT/A receptor complexes is quantitatively determined using a luminometer. In yet another aspect of this embodiment, the presence of BoNT/A toxin-FGFR3 complexes is quantitatively determined using a luminometer. In yet another aspect of this embodiment, the absence of BoNT/A toxin-FGFR3 complexes is quantitatively determined using a luminometer.

In yet another embodiment, a BoNT/A toxin is operably-linked to a peptide necessary for producing a chromogenic product. In an aspect of this embodiment, a BoNT/A toxin is operably linked to an alkaline phosphatase, a horseradish peroxidase, an urease, a β-glucourimidase, a glucose oxidase or a β-galatosidase. In another aspect of this embodiment, the presence of BoNT/A toxin-receptor complexes is quantitatively determined using a spectrophotometer. In another aspect of this embodiment, the absence of BoNT/A toxin-receptor complexes is quantitatively determined using a spectrophotometer. In another embodiment, a BoNT/A receptor is operably-linked to a peptide necessary for producing a chromogenic product. In an aspect of this embodiment, a BoNT/A receptor is operably linked to an alkaline phosphatase, a horseradish peroxidase, an urease, a β-glucourimidase, a glucose oxidase or a β-galatosidase. In another aspect of this embodiment, a FGFR3 is operably linked to a peptide necessary for producing a chromogenic product. In an aspect of this embodiment, a FGFR3 is operably linked to an alkaline phosphatase, a horseradish peroxidase, an urease, a β-glucourimidase, a glucose oxidase or a β-galatosidase. In still another aspect of this embodiment, the presence of BoNT/A toxin-BoNT/A receptor complexes is quantitatively determined using a spectrophotometer. In still another aspect of this embodiment, the absence of BoNT/A toxin-BoNT/A receptor complexes is quantitatively determined using a spectrophotometer. In yet another aspect of this embodiment, the presence of BoNT/A toxin-FGFR3 complexes is quantitatively determined using a spectrophotometer. In yet another aspect of this embodiment, the absence of BoNT/A toxin-FGFR3 complexes is quantitatively determined using a spectrophotometer.

Aspects of the present invention provide, in part, comparing the amount of toxin-receptor complexes formed in the test sample to the amount of toxin-receptor complexes formed in the control sample. In an embodiment, the amount of toxin-receptor complexes in the test sample increases as compared to the amount of toxin-receptor complexes in the control sample. In an aspect of this embodiment, an increase in the amount of toxin-receptor complexes in the test sample as compared to a positive control sample indicates a reduction or the lack of BoNT/A immunoresistance in the mammal. In an aspect of this embodiment, an increase in the amount of toxin-receptor complexes in the test sample as compared to a negative control sample indicates a reduction or the lack of BoNT/A immunoresistance in the mammal. In another embodiment, the amount of toxin-receptor complexes in the test sample decreases as compared to the amount of toxin-receptor complexes in the control sample. In an aspect of this embodiment, a decrease in the amount of toxin-receptor complexes in the test sample as compared to a positive control sample indicates BoNT/A immunoresistance in the mammal or increase thereof. In another aspect of this embodiment, a decrease in the amount of toxin-receptor complexes in the test sample as compared to a negative control sample indicates BoNT/A immunoresistance in the mammal or increase thereof.

In another embodiment, the amount of toxin-receptor complexes present in the test sample is compared to the amount of toxin-receptor complexes present in the control sample. In an aspect of this embodiment, an increase in the presence of toxin-receptor complexes in the test sample as compared to a positive control sample indicates a reduction or the lack of BoNT/A immunoresistance in the mammal. In an aspect of this embodiment, an increase in the presence of toxin-receptor complexes in the test sample as compared to a negative control sample indicates a reduction or the lack of BoNT/A immunoresistance in the mammal. In another aspect of this embodiment, a decrease in the presence of toxin-receptor complexes in the test sample as compared to a positive control sample indicates BoNT/A immunoresistance in the mammal or increase thereof. In another aspect of this embodiment, a decrease in the presence of toxin-receptor complexes in the test sample as compared to a negative control sample indicates BoNT/A immunoresistance in the mammal of increase thereof.

In another embodiment, the amount of toxin-receptor complexes absent in the test sample is compared to the amount of toxin-receptor complexes absent in the control sample. In an aspect of this embodiment, an increase in the absence of toxin-receptor complexes in the test sample as compared to a positive control sample indicates BoNT/A immunoresistance in the mammal or increase thereof. In another aspect of this embodiment, an increase in the absence of toxin-receptor complexes in the test sample as compared to a negative control sample indicates BoNT/A immunoresistance in the mammal or increase thereof. In another aspect of this embodiment, a decrease in the absence of toxin-receptor complexes in the test sample as compared to a positive control sample indicates a reduction or the lack of BoNT/A immunoresistance in the mammal. In another aspect of this embodiment, a decrease in the absence of toxin-receptor complexes in the test sample as compared to a negative control sample indicates a reduction or the lack of BoNT/A immunoresistance in the mammal.

Aspects of the present invention provide, in part, comparing the amount of free receptor in the test sample to the amount of free receptor formed in the control sample. In an embodiment, the amount of free receptor in the test sample increases as compared to the amount of free receptor in the control sample. In an aspect of this embodiment, an increase in the amount of free receptor in the test sample as compared to a positive control sample indicates BoNT/A immunoresistance in the mammal or increase thereof. In an aspect of this embodiment, an increase in the amount of free receptor in the test sample as compared to a negative control sample indicates BoNT/A immunoresistance in the mammal or increase thereof. In another embodiment, the amount of free receptor in the test sample decreases as compared to the amount of free receptor complexes in the control sample. In an aspect of this embodiment, a decrease in the amount of free receptor in the test sample as compared to a positive control sample indicates a reduction or the lack of BoNT/A immunoresistance in the mammal. In another aspect of this embodiment, a decrease in the amount of free receptor in the test sample as compared to a negative control sample indicates a reduction or the lack of BoNT/A immunoresistance in the mammal.

In another embodiment, the amount of free receptor present in the test sample is compared to the amount of free receptor present in the control sample. In an aspect of this embodiment, an increase in the presence of free receptor in the test sample as compared to a positive control sample indicates BoNT/A immunoresistance in the mammal or increase thereof. In an aspect of this embodiment, an increase in the presence of free receptor in the test sample as compared to a negative control sample indicates BoNT/A immunoresistance in the mammal or increase thereof. In another aspect of this embodiment, a decrease in the presence of free receptor complexes in the test sample as compared to a positive control sample indicates a reduction or the lack of BoNT/A immunoresistance in the mammal. In another aspect of this embodiment, a decrease in the presence of free receptor complexes in the test sample as compared to a negative control sample indicates a reduction or the lack of BoNT/A immunoresistance in the mammal.

In another embodiment, the amount of free receptor absent in the test sample is compared to the amount of free receptor absent in the control sample. In an aspect of this embodiment, an increase in the absence of free receptor in the test sample as compared to a positive control sample indicates a reduction or the lack of BoNT/A immunoresistance in the mammal. In another aspect of this embodiment, an increase in the absence of free receptor in the test sample as compared to a negative control sample indicates a reduction or the lack of BoNT/A immunoresistance in the mammal. In another aspect of this embodiment, a decrease in the absence of free receptor in the test sample as compared to a positive control sample indicates BoNT/A immunoresistance in the mammal or increase thereof. In another aspect of this embodiment, a decrease in the absence of free receptor in the test sample as compared to a negative control sample indicates BoNT/A immunoresistance in the mammal or increase thereof.

Aspects of the present invention provide, in part, comparing the amount of toxin-antibody complexes formed in the test sample to the amount of toxin-antibody complexes formed in the control sample. In an embodiment, the amount of toxin-antibody complexes in the test sample increases as compared to the amount of toxin-antibody complexes in the control sample. In an aspect of this embodiment, an increase in the amount of toxin-antibody complexes in the test sample as compared to a positive control sample indicates BoNT/A immunoresistance in the mammal or increase thereof. In an aspect of this embodiment, an increase in the amount of toxin-antibody complexes in the test sample as compared to a negative control sample indicates BoNT/A immunoresistance in the mammal or increase thereof. In another embodiment, the amount of toxin-antibody complexes in the test sample decreases as compared to the amount of toxin-antibody complexes in the control sample. In an aspect of this embodiment, a decrease in the amount of toxin-antibody complexes in the test sample as compared to a positive control sample indicates a reduction or the lack of BoNT/A immunoresistance in the mammal. In another aspect of this embodiment, a decrease in the amount of toxin-antibody complexes in the test sample as compared to a negative control sample indicates a reduction or the lack BoNT/A immunoresistance in the mammal.

In another embodiment, the amount of toxin-antibody complexes present in the test sample is compared to the amount of toxin-antibody complexes present in the control sample. In an aspect of this embodiment, an increase in the presence of toxin-antibody complexes in the test sample as compared to a positive control sample indicates BoNT/A immunoresistance in the mammal or increase thereof. In an aspect of this embodiment, an increase in the presence of toxin-antibody complexes in the test sample as compared to a negative control sample indicates BoNT/A immunoresistance in the mammal or increase thereof. In another aspect of this embodiment, a decrease in the presence of toxin-antibody complexes in the test sample as compared to a positive control sample indicates a reduction or the lack of BoNT/A immunoresistance in the mammal. In another aspect of this embodiment, a decrease in the presence of toxin-antibody complexes in the test sample as compared to a negative control sample indicates a reduction or lack of BoNT/A immunoresistance in the mammal.

In another embodiment, the amount of toxin-antibody complexes absent in the test sample is compared to the amount of toxin-antibody complexes absent in the control sample. In an aspect of this embodiment, an increase in the absence of toxin-antibody complexes in the test sample as compared to a positive control sample indicates a reduction or the lack of BoNT/A immunoresistance in the mammal.

In another aspect of this embodiment, an increase in the absence of toxin-antibody complexes in the test sample as compared to a negative control sample indicates a reduction or the lack of BoNT/A immunoresistance in the mammal. In another aspect of this embodiment, a decrease in the absence of toxin-antibody complexes in the test sample as compared to a positive control sample indicates BoNT/A immunoresistance in the mammal or increase thereof. In another aspect of this embodiment, a decrease in the absence of toxin-antibody complexes in the test sample as compared to a negative control sample indicates BoNT/A immunoresistance in the mammal or increase thereof.

It is envisioned that any and all assay conditions suitable for detecting the present of a neutralizing anti-BoNT/A antibody in a sample are useful in the methods disclosed in the present specification, such as, e.g., linear assay conditions and non-linear assay conditions. In an embodiment of the present invention, the assay conditions are linear. In an aspect of this embodiment, the assay amount of BoNT/A toxin is in excess. In another aspect of this embodiment, the assay amount of a receptor is rate-limiting. In another aspect of this embodiment, the assay amount of a sample is rate-limiting.

In an embodiment, all steps of a method are performed in solution. However, it is also envisioned that aspects of the present invention can optionally attach an assay component to a solid or insoluble material. Such a solid support can be, without limitation, e.g., a tube; plate; pins or “dipsticks”, column; particle, bead or other spherical or fibrous chromatographic media, such as, e.g., agarose beads, sepharose beads, silica beads and plastic beads; sheets or membranes, such as, e.g., nitrocellulose and polyvinylidene fluoride (PVDF). The solid support selected can have a physical property that renders it readily separable from soluble or unbound material and generally allows unbound materials, such as, e.g., excess reagents, reaction by-products, or solvents, to be separated or otherwise removed (by, e.g., washing, filtration, centrifugation, etc.) from solid support-bound assay component. Non-limiting examples of how to make and use a solid support-bound assay component are described in, e.g., MOLECULAR CLONING, A LABORATORY MANUAL, supra, (2001); and CURRENT PROTOCOLS IN MOLECULAR BIOLOGY, supra, (2004).

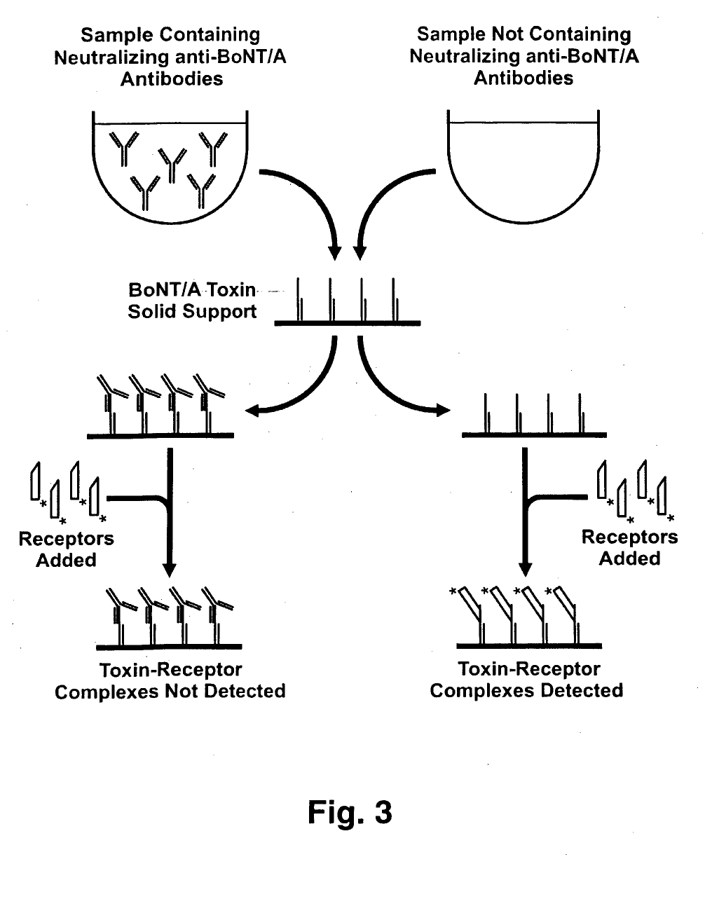

In one embodiment, a BoNT/A toxin disclosed in the present specification can optionally be attached to a solid or insoluble material (see, e.g., FIG. 3). In another embodiment, a BoNT/A receptor, such as, e.g., a FGFR3 and members of the synaptotagmin family, disclosed in the present specification can optionally be attached to a solid or insoluble material.

As a general procedure, because the exact amount of a BoNT/A toxin can be readily determined by one skilled in the art, the assay amounts of a sample and a BoNT/A receptor can be determined based on a fixed assay amount of a BoNT/A toxin.

In an embodiment, it is envisioned that detection of any and all binding levels of a BoNT/A receptor to a BoNT/A toxin capable of being detected by a detection method disclosed in the present specification are useful in aspects of the present invention. Thus, aspects of this embodiment may include detection of, e.g., at least 10% binding of a BoNT/A toxin to a BoNT/A receptor, at least 20% binding of a BoNT/A toxin to a BoNT/A receptor, at least 30% binding of a BoNT/A toxin to a BoNT/A receptor, at least 40% binding of a BoNT/A toxin to a BoNT/A receptor, at least 50% binding of a BoNT/A toxin to a BoNT/A receptor, at least 60% binding of a BoNT/A toxin to a BoNT/A receptor, at least 70% binding of a BoNT/A toxin to a BoNT/A receptor, at least 80% binding of a BoNT/A toxin to a BoNT/A receptor, or at least 90% binding of a BoNT/A toxin to a BoNT/A receptor. To ascertain an appropriate assay amount of a BoNT/A receptor in this embodiment, the binding capacity of a BoNT/A receptor preparation towards a BoNT/A toxin is determined using a fixed amount of a BoNT/A toxin and a range of BoNT/A receptor amounts in order to generate a toxin-receptor binding saturation curve. This protocol is routine procedure well within the scope of one skilled in the art and from the teaching herein.

In another embodiment of the present invention, it is foreseen that detection of any and all binding levels of neutralizing anti-BoNT/A antibody to a BoNT/A toxin capable of being detected by a detection means disclosed in the present specification are useful in aspects of the present invention. Thus aspects of this embodiment may include detection of, e.g., at least 10% binding of a BoNT/A toxin with a neutralizing anti-BoNT/A antibody, at least 20% binding of a BoNT/A toxin with a neutralizing anti-BoNT/A antibody, at least 30% binding of a BoNT/A toxin, at least 40% binding of a BoNT/A toxin with a neutralizing anti-BoNT/A antibody, at least 50% binding of a BoNT/A toxin with a neutralizing anti-BoNT/A antibody, at least 60% binding of a BoNT/A toxin with a neutralizing anti-BoNT/A antibody, at least 70% binding of a BoNT/A toxin with a neutralizing anti-BoNT/A antibody, at least 80% binding of a BoNT/A toxin with a neutralizing anti-BoNT/A antibody, or at least 90% binding of a BoNT/A toxin with a neutralizing anti-BoNT/A antibody. To ascertain an appropriate assay amount of a sample in this embodiment, the binding inhibition of a positive control sample towards a BoNT/A toxin is determined using a fixed amount of a BoNT/A toxin and a range of sample amounts in order to generate a neutralizing anti-BoNT/A antibody binding inhibition saturation curve. This protocol is routine procedure well within the scope of one skilled in the art and from the teaching herein.

In yet another embodiment, a wide range of BoNT/A toxin amounts can be used in methods disclosed in the present specification. The assay amount of a BoNT/A toxin can be varied as appropriate by one skilled in the art and generally depends, in part, on the BoNT/A toxin being used and the detection method employed. Therefore, aspects of this embodiment may include a BoNT/A toxin amount of, e.g., at least 1 μg, at least 10 μg, at least 100 μg, at least 1 ng, at least 10 ng, at least 100 ng, at least 1 μg, or at least 10 μg. In an aspect of this embodiment, the assay amount of a BoNT/A toxin is 100 μg. In another aspect of this embodiment, the assay amount of a BoNT/A toxin is 10 ng. In another aspect of this embodiment, the assay amount of a BoNT/A toxin is 1 ng.

In yet another embodiment of the present invention, a wide range of sample volumes can be used in methods disclosed in the present specification. The assay amount of a sample can be varied as appropriate by one skilled in the art and generally depends, in part, on the amount of sample available, the BoNT/A toxin amount being used, and the detection method employed. Thus, aspects of this embodiment may include, e.g., a sample volume of at least 0.001 μL, at least 0.01 μL, at least 0.1 μL, at least, 1 μL, at least 2 μL, at least 3 μL, at least 4 μL, at least 5 μL, at least 10 μL, at least 20 μL, at least 30 μL, at least 40 μL, at least 50 μL, or at least 100 μL. In an aspect of this embodiment, the assay amount of a sample is 1 μL.

In yet another embodiment, a wide range of volumes of BoNT/A receptor-containing tissue preparations can be used in methods disclosed in the present specification. The assay volume of a BoNT/A receptor-containing tissue preparation can be varied as appropriate by one skilled in the art and generally depends, in part, on the BoNT/A receptor preparation being used, the BoNT/A toxin amount being used, and the detection method employed. Therefore, aspects of this embodiment may include a BoNT/A receptor-containing tissue preparation volume of, e.g., at least 0.001 μL, at least 0.01 μL, at least 0.1 μL, at least, 1 μL, at least 2 μL, at least 3 μL, at least 4 μL, at least 5 μL, at least 10 μL, at least 20 μL, at least 30 μL, at least 40 μL, at least 50 μL, or at least 100 μL. In an aspect of this embodiment, the assay volume of a BoNT/A receptor-containing tissue preparation is 2 μL. In another aspect of this embodiment, the assay volume of a BoNT/A receptor-containing tissue preparation is 6 μL. In another aspect of this embodiment, the assay volume of a BoNT/A receptor-containing tissue preparation is 4 μL.

In yet embodiment, a wide range of BoNT/A receptor amounts can be used in methods disclosed in the present specification. The BoNT/A receptor amount can be varied as appropriate by one skilled in the art and generally depends, in part, on the BoNT/A receptor protein being used, the BoNT/A toxin amount being used, and the detection method employed. Therefore, aspects of this embodiment may include a BoNT/A receptor amount of, e.g., at least 1 μg, at least 10 pg, at least 100 pg, at least 1 ng, at least 10 ng, at least 100 ng, at least 1 μg, or at least 10 μg. In an aspect of this embodiment, the assay amount of a BoNT/A receptor is 100 pg. In another aspect of this embodiment, the assay amount of a BoNT/A receptor is 10 ng. In another aspect of this embodiment, the assay amount of a BoNT/A receptor is 1 ng.

In still another embodiment, it is envisioned that a wide range of assay volumes can be used in methods disclosed in the present specification. Thus aspects of this embodiment may include, e.g. a volume of at least 0.001 μL, at least 0.01 μL, at least 0.1 μL, at least, 1 μL, at least 2 μL, at least 3 μL, at least 4 μL, at least 5 μL, at least 10 μL, at least 20 μL, at least 30 μL, at least 40 μL, at least 50 μL, at least 100 μL, at least 200 μL, at least 300 μL, at least 400 μL, at least 500 μL, or at least 1000 μL.

In still another embodiment, it is envisioned that any and all temperatures that allow the formation of a toxin-antibody complex or the formation of a toxin-receptor complex can be used in methods disclosed in the present specification. Assay temperatures can be varied as appropriate by one skilled in the art and generally depend, in part, on the BoNT/A toxin amount, BoNT/A receptor amount and assay time. Thus, an assay temperature should not be as low as to cause the solution to freeze and should not be as high as to denature the peptides disclosed in the present specification. In an aspect of this embodiment, the assay is performed within a temperature range above 0° C., but below 40° C. In another aspect of this embodiment, the assay is performed within a temperature range of about 4° C. to about 37° C. In yet another aspect of this embodiment, the assay is performed within a temperature range of about 2° C. to 10° C. In yet another aspect of this embodiment, the assay is performed at about 4° C. In still another aspect of this embodiment, the assay is performed within a temperature range of about 10° C. to about 18° C. In still another aspect of this embodiment, the assay is performed at about 16° C. In yet another aspect of this embodiment, the assay is performed within a temperature range of about 18° C. to about 32° C. In yet another aspect of this embodiment, the assay is performed at about 20° C. In another aspect of this embodiment, the assay is performed within a temperature range of about 32° C. to about 40° C. In another aspect of this embodiment, the assay is performed at about 37° C.

In still another embodiment, it is foreseen that any and all times sufficient for the formation of a toxin-antibody complex or the formation of a toxin-receptor complex can be used in methods disclosed in the present specification. Assay times can be varied as appropriate by one skilled in the art and generally depend, in part, on the BoNT/A toxin amount, BoNT/A receptor amount, the detection method employed and the incubation temperature. Therefore, aspects of this embodiment may include assay times of, e.g., at least 1 minute, at least 2 minutes, at least 3 minutes, at least 4 minutes, at least 5 minutes, at least 10 minutes, at least 15 minutes, at least 20 minutes, at least 25 minutes, at least 30 minutes, at least 45 minutes, at least 60 minutes, or at least 120 minutes. It is understood by one skilled in the art that an assay temperature can affect the formation of a toxin-antibody complex or the formation of a toxin-receptor complex disclosed in the present invention, and thereby can influence the length of time required to achieve sufficient complex formation. Thus, in an aspect of this embodiment, assay times of at least 45 minutes are used in an assay temperature range of about 2° C. to about 10° C. In another aspect of this embodiment, assay times of at least 30 minutes are used in an assay temperature range of about 10° C. to about 18° C. In yet another aspect of this embodiment, assay times of at least 15 minutes are used in an assay temperature range of about 18° C. to about 32° C. In another aspect of this embodiment, assay times of at least 5 minutes are used in an assay temperature range of about 32° C. to about 40° C. In another aspect of this embodiment, an assay time of 15 minutes is used at an assay temperature of about 37° C.

In a further embodiment, it is also envisioned that any and all buffers that allow the formation of a toxin-antibody complex or the formation of a toxin-receptor complex can optionally be used in methods disclosed in the present specification. Assay buffers can be varied as appropriate by one skilled in the art and generally depend, in part, on the pH value desired for the assay, the BoNT/A toxin, the BoNT/A receptor and the detection method employed. Therefore, aspects of this embodiment may optionally include, e.g., 2-amino-2-hydroxymethyl-1,3-propanediol (Tris) buffers; Phosphate buffers, such as, e.g., potassium phosphate buffers and sodium phosphate buffers; Good buffers, such as, e.g., 2-(N-morpholino) ethanesulfonic acid (MES), piperazine-N,N′-bis(2-ethanesulfonic acid) (PIPES), N,N′-bis(2-hydroxyethyl)-2-aminoethanesulfonic acid (BES), 3-(N-morpholino) propanesulfonic acid (MOPS), N-(2-hydroxyethyl) piperazine-N′-(2-ethanesulfonic acid) (HEPES), piperazine-N,N′-bis(2-hydroxypropanesulfonic acid) (POPSO), N-tris(hydroxymethyl) methylglycine (Tricine), N-tris(hydroxymethyl)methyl-3-aminopropanesulfonic acid (TAPS), 3-[(1,1-dimethyl-2-hydroxyethyl)amino]-2-hydroxypropanesulfonic acid (AMPSO), 3-(cyclohexylamino)-2-hydroxy-1-propanesulfonic acid (CAPSO), and 3-(cyclohexylamino)-1-propanesulfonic acid (CAPS); saline buffers, such as, e.g., Phosphate-buffered saline (PBS), HEPES-buffered saline, and Tris-buffered saline (TBS); Acetate buffers, such as, e.g., magnesium acetate, potassium actetate, and Tris acetate; and the like, or any combination thereof. In addition, the buffer concentration in a method disclosed in the present specification can be varied as appropriate by one skilled in the art and generally depend, in part, on the buffering capacity of a particular buffer being used and the detection means employed. Thus, aspects of this embodiment may include a buffer concentration of, e.g., at least 1 mM, at least 5 mM, at least 10 mM, at least 20 mM, at least 30 mM, at least 40 mM, at least 50 mM, at least 60 mM, at least 70 mM, at least 80 mM, at least 90 mM, or at least 100 mM. Non-limiting examples of how to make and use specific buffers are described in, e.g., MOLECULAR CLONING, A LABORATORY MANUAL, supra, (2001); and CURRENT PROTOCOLS IN MOLECULAR BIOLOGY, supra, (2004).

In a further embodiment, it is also envisioned that any and all salts that allow the formation of a toxin-antibody complex or the formation of a toxin-receptor complex can optionally be used in methods disclosed in the present specification. Assay salts can be varied as appropriate by one skilled in the art and generally depend, in part, on the physiological conditions desired for the assay, the BoNT/A toxin, the BoNT/A receptor and the detection method employed. Therefore, aspects of this embodiment may optionally include, e.g., sodium chloride, potassium chloride, calcium chloride, magnesium chloride, manganese chloride, zinc chloride, magnesium sulfate, zinc sulfate, and the like, or any combination thereof. In addition, the salt concentration in a method disclosed in the present specification can be varied as appropriate by one skilled in the art and generally depend, in part, on the buffering capacity of a particular buffer being used and the detection means employed. Thus, aspects of this embodiment may include a salt concentration of, e.g., at least 1 mM, at least 5 mM, at least 10 mM, at least 20 mM, at least 30 mM, at least 40 mM, at least 50 mM, at least 60 mM, at least 70 mM, at least 80 mM, at least 90 mM, or at least 100 mM. Non-limiting examples of how to make and use specific salts are described in, e.g., MOLECULAR CLONING, A LABORATORY MANUAL, supra, (2001); and CURRENT PROTOCOLS IN MOLECULAR BIOLOGY, supra, (2004).

In a further embodiment, it is also envisioned that any and all enhancing agents that allow the formation of a toxin-antibody complex or the formation of a toxin-receptor complex can optionally be used in methods disclosed in the present specification. Assay enhancing agents can be varied as appropriate by one skilled in the art and generally depend, in part, on the assay conditions desired for the assay, the BoNT/A toxin, the BoNT/A receptor and the detection method employed. Therefore, aspects of this embodiment may optionally include, e.g., stabilizing agents including proteins, such as, e.g., bovine serum albumin and milk proteins, such as, e.g., casien, thyroglobulin, fetuin, asialofetuin, cytochrome c and bovine submaxillary mucin and polyamines, such as, e.g., spermidine and spermine; chelating agents including, e.g., ethylenediamine tetraacetic acid (EDTA) and ethylene glycol bis(β-aminoethyl ether) N,N,N′,N′-tetraacetic acid (EGTA); reducing agents, including, e.g., β-mercaptoethanol and dithiothreitol (DTT); dimethylsulfoxide (DMSO); and the like, or any combination thereof. In addition, the enhancing agent concentration in a method disclosed in the present specification can be varied as appropriate by one skilled in the art and generally depend, in part, on the assay conditions desired for the assay and the detection means employed. In an aspect of this embodiment, concentrations for a stabilizing agent may include, e.g., at least 10 μg/mL, at least 50 μg/mL, at least 100 μg/mL, at least 200 μg/mL or at least 500 μg/mL. In another aspect of this embodiment, concentrations for a chelating agent may include, e.g., at least 10 nM, at least 50 nM, at least 100 nM, at least 500 nM, at least 1 mM or at least 10 mM. In yet another aspect of this embodiment, concentrations for a reducing agent may include, e.g., at least 10 nM, at least 50 nM, at least 100 nM, at least 500 nM, at least 1 mM, at least 10 mM or at least 100 mM. Non-limiting examples of how to make and use specific enhancing agents are described in, e.g., MOLECULAR CLONING, A LABORATORY MANUAL, supra, (2001); and CURRENT PROTOCOLS IN MOLECULAR BIOLOGY, supra, (2004).

In an additional embodiment of the invention, it is also foreseen that a wide variety of processing formats can be used in conjunction with the methods of the present invention, including, without limitation, manual processing, partial automated-processing, semi-automated-processing, full automated-processing, high throughput processing, high content processing, and the like or any combination thereof.

It is understood by one skilled in the art that a wide variety of factors can influence assay conditions, including, without limitation, solution variations, buffer variations, reagent variations, equipment variations and facility variations. Thus, any particular assay condition selected by one skilled in the art will require routine experimentation in order to optimize the method to account for such factors. These optimization protocols are routine procedures well within the scope of one skilled in the art and the teaching herein.

### EXAMPLES

The following non-limiting examples are provided for illustrative purposes only in order to facilitate a more complete understanding of disclosed embodiments and are in no way intended to limit any of the embodiments disclosed in the present invention.

### Example 1

### Isolation of Synaptosome Preparation Containing BoNT/A Receptors

To prepare a crude synaptosome preparation, approximately 10 g, wet weight of mouse brain tissue was homogenized in 9 volumes of ice-cold Tris-buffered sucrose (10 mM 2-amino-2-hydroxymethyl-1,3-propanediol hydrochloric acid, pH 7.0 (Tris-HCl, pH 7.0); 320 mM sucrose) using a motor-driven, glass-teflon homogenizer (800 rpm for 10 strokes). The homogenate was centrifuged (1000×g at 4° C. for 10 minutes) to pellet the cellular debris and nuclei fraction (P1). The resulting supernatant was then centrifuged (26,000×g at 4° C. for 17 minute) to yield the crude synaptosome pellet (P2). The synaptosome fraction was then washed by resuspending the P2 pellet in 10 volumes of ice-cold Tris-buffered sucrose and centrifuging the fraction (10,000×g at 4° C. for 15 minute). The resulting washed crude synaptosome fraction (P2′) was resuspended in an appropriate volume of buffer depending on the subsequent step (i.e., further purification or use in BoNT/A Immunoresistant Assay). For BoNT/A Immunoresistant Assays, the P2′ pellet was resuspended in an appropriate volume of Tris-sodium buffer, pH7.0 (10 mM Tis-HCl, pH 7.0; 140 mM sodium chloride) and stored at −80° C. until needed.

To prepare a purified synaptosome preparation, the P2′ pellet containing the washed crude synaptosome fraction described above was resuspended in 8.0 mL of Tris-buffered sucrose, layered onto 4.0 mL of a discontinuous gradient, comprising a 0.8 M sucrose top layer and a 1.2 M sucrose bottom layer, and centrifuged in swinging bucket rotor (26,000×g at 4° C. for 210 minutes). The interphase fraction between the 0.8 M and 1.2 M sucrose layers was collected and the resulting purified synaptosome fraction (P2″) was resuspended in an appropriate volume of a Tris-sodium buffer, pH 7.0. Purified synaptosome preparations were stored at −80° C. until needed.

The same procedure is used to purify synaptosomes from a cell line, except, cells from an appropriate cell line are substituted for the mouse brain tissue.

### Example 2

### Isolation of Synaptic Vesicles, Synaptic Membranes and Synaptic Densities Containing BoNT/A Receptors

To prepare a synaptic vesicle preparation, the P2′ pellet containing the washed crude synaptosome fraction described above is lysed by hypoosmotic shock in 9 volumes ice cold deionized, distilled water containing protease and phosphatase inhibitors and three strokes of a glass-teflon homogenizer. The lysed solution is adjusted to 10 mM Tris by adding 1 M Tris-HCl, pH 7.0 (approximately 1/250 volume) and mixed continuously at 4° C. for 30 min to ensure complete lysis. The buffered lysate solution is centrifuged (25,000×g at 4° C. for 20 minute) to yield a supernatant (S3, crude synaptic vesicle fraction) and a pellet (P3, lysed synaptic membrane fraction). The S3 fraction is centrifuge (165,000×g at 4° C. for 2 hour) to pellet the synaptic vesicle fraction. The resulting pellet is resuspended in an appropriate volume of Tris-sodium buffer, pH 7.0 containing protease and phosphatase inhibitors to yield the purified synaptic vesicle preparation. Synaptic vesicle preparations are stored at −80° C. until needed.

To prepare a synaptic membrane preparation, the P3 pellet containing the lysed synaptic membrane fraction described above is resuspend in 8.0 mL of Tris-buffered sucrose, layered onto a discontinuous gradient comprising a 0.8 M sucrose top layer, a 1.0 M sucrose middle layer and a 1.2 M sucrose bottom layer, and centrifuged in a swinging bucket rotor (150,000×g at 4° C. for 2 hour). The interphase fraction between the 1.0 M and 1.2 M sucrose layers is collected and the sucrose molarity of the solution adjusted to 320 mM sucrose by adding an appropriate volume of ice-cold 10 mM Tris (approximately 2.5 volumes). The adjusted interface fraction is centrifuged in a swinging bucket rotor (150,000×g at 4° C. for 30 minute) to yield the purified synaptic membrane pellet (P3′). The resulting purified synaptic membrane fraction (P3′) is resuspended in an appropriate volume of buffer depending on the subsequent step (i.e., further purification or use in BoNT/A Immunoresistant Assay). For BoNT/A Immunoresistant Assays, the P3′ pellet is resuspended in an appropriate volume Tris-sodium buffer, pH 7.0 containing protease and phosphatase inhibitors are stored at −80° C. until needed.

To prepare a synaptic density preparation, the P3′ pellet containing the purified synaptic membrane fraction described above is resuspended in 4.0 mL of ice cold 10 mM Tris-HCl, pH7.0. An appropriate volume of Triton X-100 (4-octylphenol polyethoxylate) is then added to a final concentration of to 0.5% (v/v) and this mixture is mixed by rotation at 4° C. for 15 minutes. The mixed solution is centrifuged (32,000×g at 4° C. for 20 min) to yield the synaptic density pellet (P4). The P4 pellet is resuspended in 3.0 mL of ice cold 10 mM Tris-HCl, pH7.0. An appropriate volume of Triton X-100 (4-octylphenol polyethoxylate) is added to a final concentration of to 0.5% (v/v) and the resuspended pellet is mixed by rotation at 4° C. for 15 minutes. The mixed solution is centrifuged (200,000×g at 4° C. for 20 min) to yield the purified synaptic density pellet (P4′). The resulting pellet is resuspended in an appropriate volume of 100 mM Ringer's solution, pH 7.0 containing protease and phosphatase inhibitors to yield the purified synaptic density preparation. Synaptic density preparations are stored at −80° C. until needed.

### Example 3

### Isolation of Serum Sample

To prepare a serum sample, 5-10 mL of whole blood was drawn in a serum separator tube. The blood was allowed to clot by incubating at 4° C. for 60 minute. The clotted blood was centrifuged (10,000×g at 4° C. for 10 minutes) to pellet the debris. The resulting serum sample (i.e., the supernatant) was dispensed into 50 μl aliquots and stored at −20° C. until needed.

### Example 4

### Assay Conditions

To establish assay conditions for a BoNT/A Immunoresistant Assay, binding parameters for 1) a BoNT/A toxin to a BoNT/A receptor and 2) neutralizing anti-BoNT/A antibodies to a BoNT/A toxin were determined.

To make radioactively-labeled active BoNT/A toxin, active BoNT/A (Metabiologics, Inc., Madison, Wis.) was labeled with ¹²⁵Iodine using a chloramine T method as described in, e.g. W. M Hunter & F. C. Greenwood, Preparation of Iodine-131-labeled human growth hormone of high specific activity, 194 NATURE 495-496, (1962). A labeling reaction comprising 50 μl of 100 mM potassium phosphate, pH 8.0 containing 1.0 μg active BoNT/A toxin, 5 μl of 10 mCi/mL sodium ¹²⁵Iodine, and 25 μl of 100 mM potassium phosphate, pH 8.0 containing 2 mg/mL chloramine T was incubated on ice for 5 minutes. To this labeling reaction, 50 μl of 100 mM potassium phosphate, pH 8.0 containing 20 mg/mL sodium metabisulfite was added to stop the reaction. Excess unlabeled radioactive iodine was removed from the ¹²⁵Iodine-labeled toxin by applying the labeling mixture through a Sephadex G-25 gel filtration column equilibrated and eluted as a single fraction with a column solution comprising 10 mM phosphate-buffered saline, pH 7.2; 150 mM sodium chloride; and 0.1% bovine serum albumin (BSA). The level of ¹²⁵Iodine incorporation was determined by measuring the radioactivity from a 1 μL aliquot using a gamma scintillation counter. The ¹²⁵Iodine-labeled BoNT/A toxin containing eluent was adjusted to a radioactivity level suitable for the BoNT/A Immunoresistant Assay. The ¹²⁵Iodine-labeled active BoNT/A toxin was stored at 4° C. and used within two days.

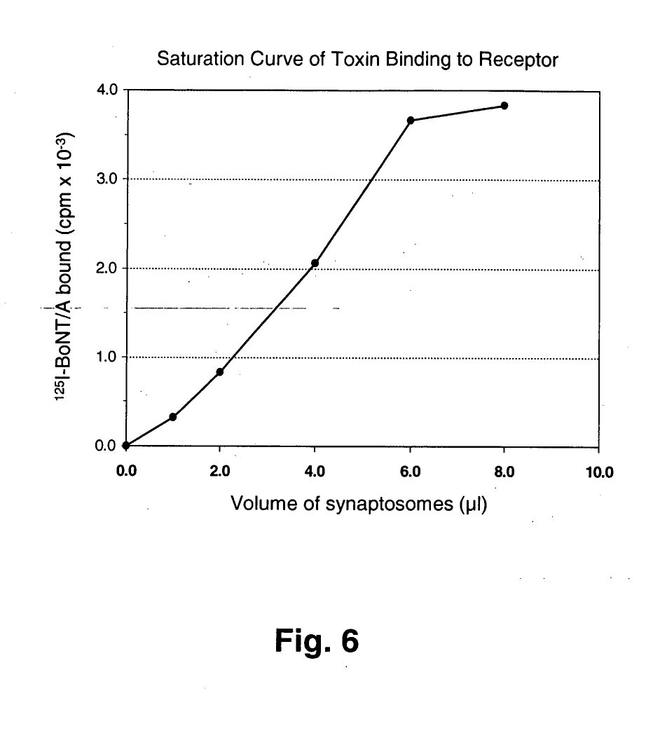

To determine the amount of receptor required to achieve saturation binding with a fixed amount of BoNT/A toxin, a BoNT/A receptor binding assay was conducted. Approximately 50,000 counts/minute of ¹²⁵I-labeled active BoNT/A toxin was mixed with increasing volumes of a synaptosome preparation (from 0 to 8 μL) in 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% (w/v) bovine serum albumin). The reaction mixtures were incubated at 37° C. for 20 minutes in order to allow for the formation of any toxin-receptor complexes. The reaction mixtures were then microcentrifuged (23,000×g at 20° C. for 3 minutes) to pellet toxin-receptor complexes. The pellets were washed twice in 800 μL of Ringer's solution, pH 7.0 to remove any unbound toxin. The pellets containing toxin-receptor complexes were resuspended in 300 μL of Ringer's solution, pH 7.0, transferred to a glass scintillation tube, and the amount of radioactivity from these complexes measured using a gamma scintillation counter. The experiment was carried out in triplicate. The formation of toxin-receptor complexes increased until a plateau was reached at about when 6 μL of synaptosomes was added to the reaction (FIG. 6).

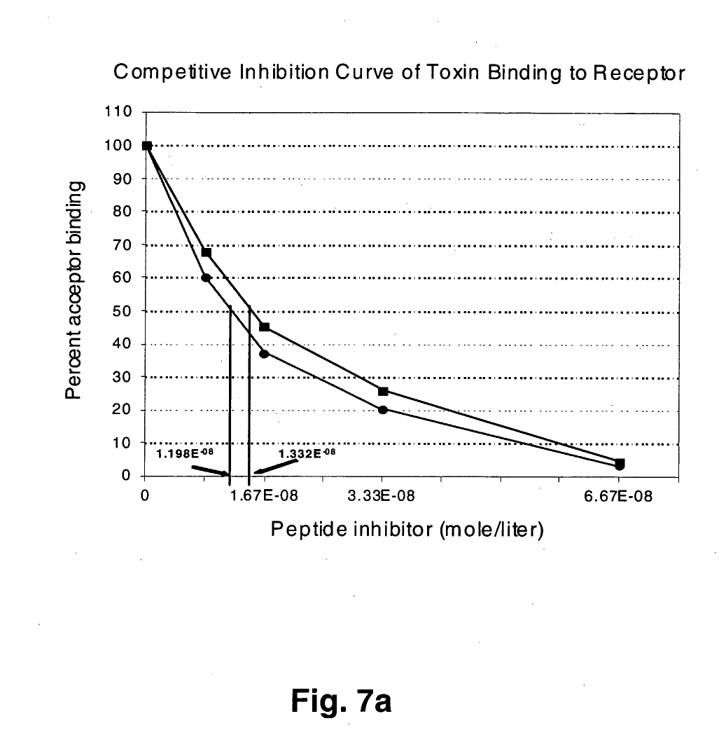

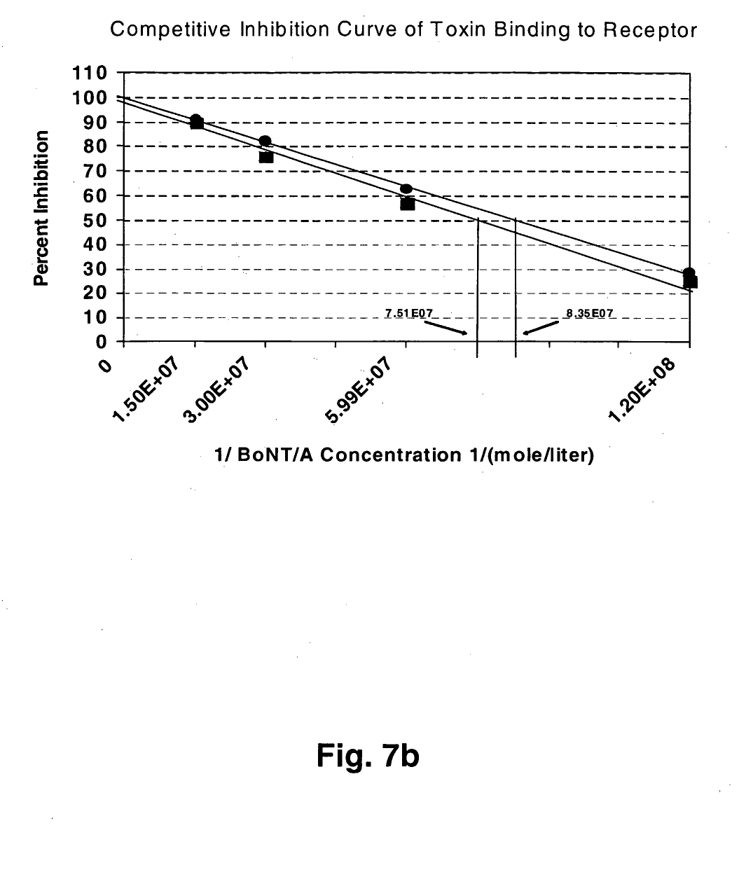

To determine the binding specificity of a BoNT/A toxin for a BoNT/A receptor, a competitive binding inhibition assay was conducted using unlabeled BoNT/A toxin as the competitive inhibitor. Approximately 50,000 counts/minute (about 1 ng) of ¹²⁵I-labeled active BoNT/A toxin was allowed to bind to 4 μL of synaptosomes in the presence of different amounts of either unlabeled active BoNT/A toxin or unlabeled inactive BoNT/A toxin. The reaction mixtures were incubated at 37° C. for 5 minutes in order to allow for the formation of any toxin-receptor complexes. The reaction mixtures were then microcentrifuged (23,000×g at 20° C. for 3 minutes) to pellet toxin-receptor complexes. The pellets were washed twice in 800 μL of Ringer's solution, pH 7.0 to remove any unbound toxin. The pellets containing toxin-receptor complexes were resuspended in 300 μL of Ringer's solution, pH 7.0, transferred to a glass scintillation tube, and the amount of radioactivity from these complexes measured using a gamma scintillation counter. The experiment was carried out in triplicate. The binding of ¹²⁵I-labeled active BoNT/A toxin to a BoNT/A receptor decreased steadily in the presence of increasing amounts of either unlabeled BoNT/A toxin (FIGS. 7a & 7b). The binding was completely (100%) inhibited by the unlabeled BoNT/A toxins, but not by unrelated proteins indicating that the binding of ¹²⁵I-labeled BoNT/A to receptor-containing synaptosome preparation was entirely specific. The 50% inhibition value (IC₅₀) for unlabeled active BoNT/A was at a concentration of approximately 1.2×10⁻⁸ M, while the IC50 for unlabeled inactive BoNT/A was 1.3×10⁻⁸ M (FIG. 7a). These experiments also indicated that 2.0 μg of unlabelled inactive BoNT/A toxin inhibited about 98% of the ¹²⁵I-labeled active BoNT/A binding to synaptosomes, while 2.0 μg of unlabeled active BoNT/A prevented approximately 78% of the ¹²⁵I-labeled inactive BoNT/A binding to synaptosomes.

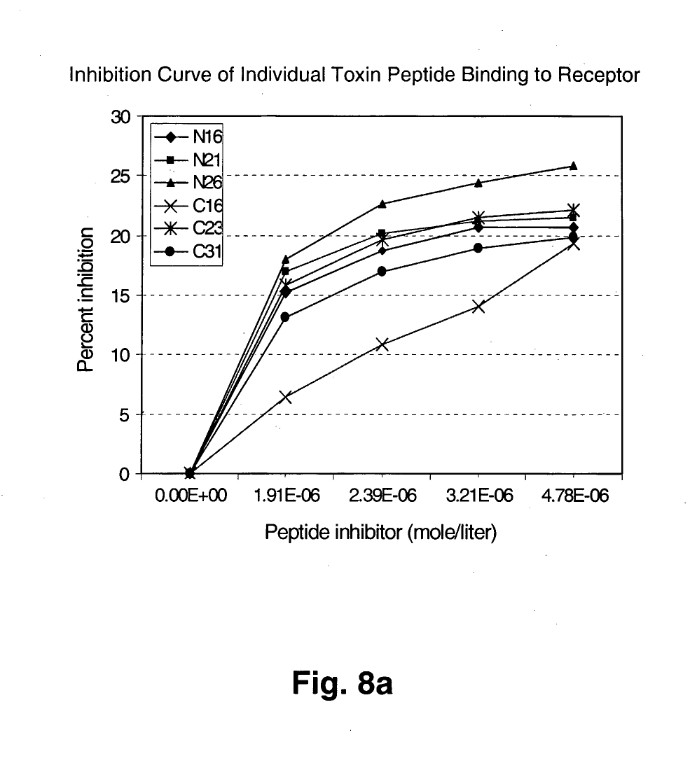

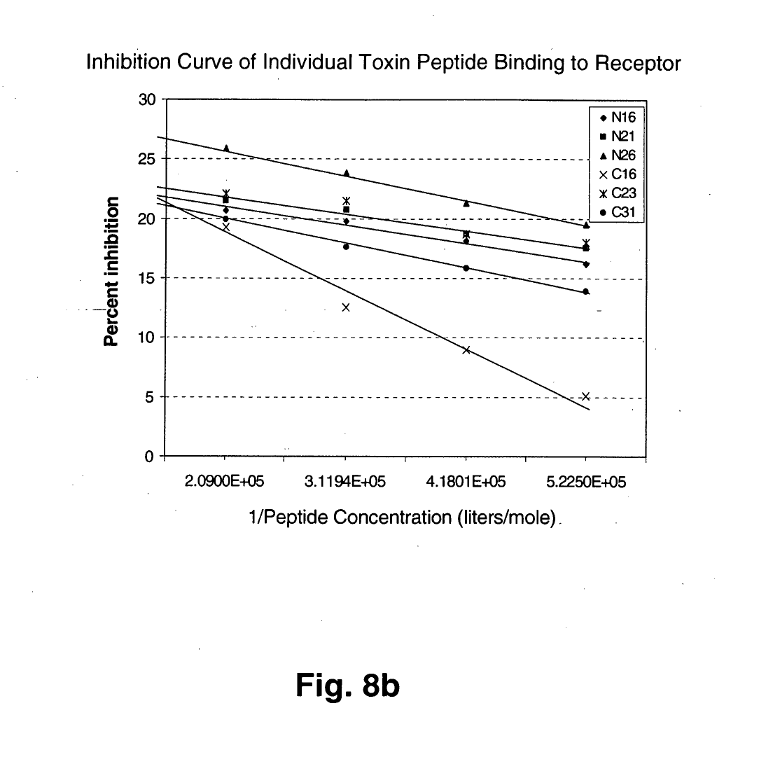

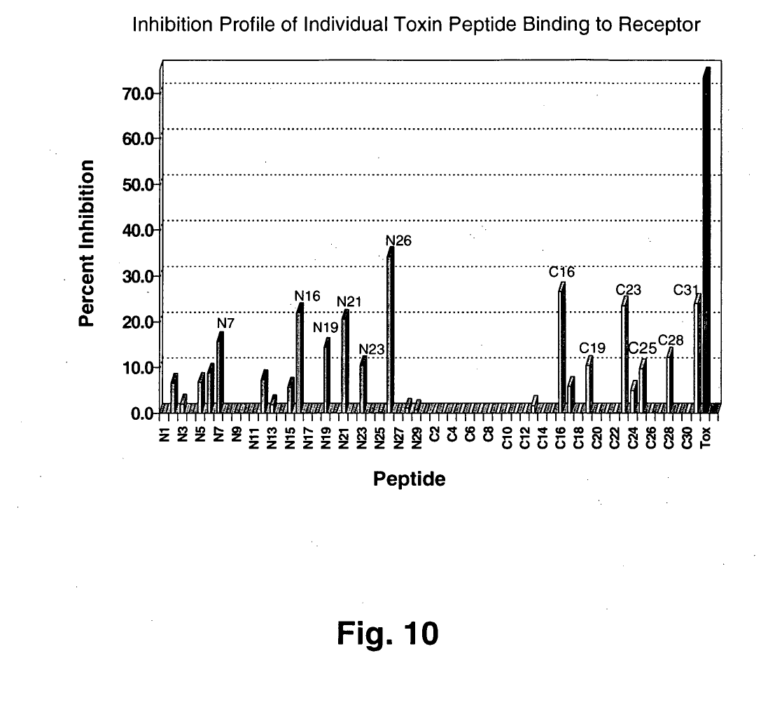

Additional competitive inhibition assays were performed with synthetic H-chain peptides. The assays were performed as described above, except that unlabeled H-chain synthetic peptides were added as the competitive inhibitor. FIG. 8 shows an example of the inhibition curves obtained with specific peptides. The values of maximum inhibition were obtained by plotting the inhibition values against the reciprocal of the different peptide concentrations used in the inhibition assay (FIG. 8b). The maximum inhibitory activities of the 60 peptides showed that the receptor-binding regions were present, as expected, on the H_{C} domain (FIG. 10). However, a number of such regions were also found on the H_{N} domain (FIG. 10). On the H_{N} domain, inhibitory activities greater than 10% were exhibited, in decreasing order, by peptides N26 (33.4%), N21 (25.0%), N16 (23.2%), N7 (15.7%), N19 (14.4%), and N23 (10.3%). Five other peptides, N2, N5, N6, N12 and N15, possessed inhibitory activities between 5.6-8.7%. The remaining 18 H_{N} peptides had little or no detectable inhibitory activity. In the H_{C} domain, regions within peptides C16, C23 and C31 had the highest inhibitory activities (between 25-29%), followed in inhibitory activity (10-12%) by peptides C19, C25 and C28. Two other peptides, C17 and C24, had very low inhibitory activities (5.8 and 4.9%, respectively). The remaining 23 H_{C} peptides had little or no detectable inhibitory activity. It should be noted that the inhibiting peptides of H_{N} and H_{C} required two orders of magnitude more peptide (greater than 5.0×10⁻⁶ M) than intact BoNT/A toxin to achieve maximum inhibition and they showed only insignificant differences in their affinities.

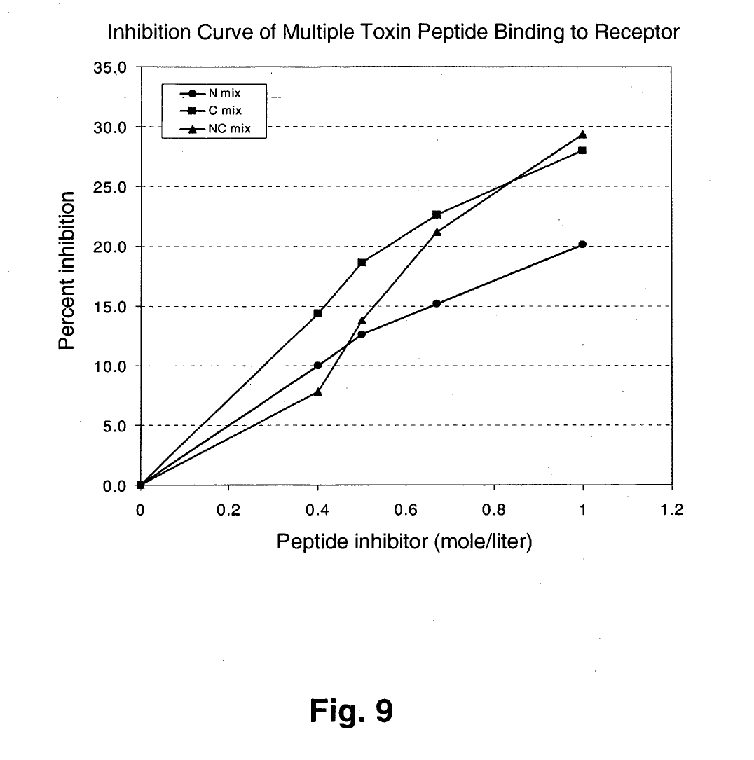

The competitive inhibitory activities were also determined for mixtures containing equimolar quantities of various H_{C} and H_{N} synthetic peptides. For example, the following peptide mixtures were used as the competitive inhibitor peptide source: (1) The six H_{N} peptides N7, N16, N19, N21, N23 and N26 in a mixture containing 0.167 μg of each peptide in 100 μL of reaction mixture; (2) the five H_{C} peptides C16, C19, C23, C28 and C31 in a mixture containing 0.200 μg of each peptide in 100 μL of reaction mixture; (3) all eleven H_{N}/Hc peptides N7, N16, N19, N21, N23, N26, C16, C19, C23, C28 and C31 in a mixture containing 0.091 μg of each peptide. In these experiments the amounts of inhibiting peptide mixture used were increased up to 1 μg/100 μL of each reaction mixture. Under these conditions, the controls that did not have receptor showed no non-specific binding of ¹²⁵I-labeled BoNT/A toxin to the peptides. When higher amounts of peptide mixture were used, some non-specific binding of ¹²⁵I-labeled BoNT/A toxin to the peptides was observed, which increased with the amount of peptide mixture and thus afforded unreliable inhibition values. In addition, the inhibitory capability of each peptide was determined individually FIG. 9 and Table 1 show the inhibitory activities of the three mixtures.

TABLE 1
Inhibitory activities of equimolar mixtures of the active peptides

| | Percent Inhibition^{a} | |
|---|---|---|
| | Sum of individual peptide | Peptide mixture |
| Inhibitor mixture | inhibition (%) | inhibition (%) |
| H_{N} peptides | 31.2 | 30.1 |
| (0.167 μg/peptide) | | |
| H_{C} peptides | 28.4 | 37.4 |
| (0.200 μg/peptide) | | |
| H_{N/}H_{C} peptides | 31.2 | 44.8 |
| (0.091 μg/peptide) | | |

^{a}The inhibition of the six H_{N} peptides was determined individually at 0.167 μg or in a mixture containing 0.167 μg of each peptide in 100 μL of reaction mixture; the inhibition of the five H_{C} peptides was determined individually at 0.200 μg or in a mixture containing 0.200 μg of each peptide in 100 μL of reaction mixture; and the inhibition of the six H_{N} peptides and the five H_{C} peptides was determined individually at 0.091 μg or in a mixture containing 0.091 μg of each peptide in 100 μL of reaction mixture.

The mixture of the H_{N} peptides contained at maximum amount 0.167 μg of each of the six peptides and exhibited a maximum inhibitory activity of 30.1%. At this excess, the sum of the inhibition of the six peptides is expected to be 31.2%. This compares very well with the inhibition exerted by a mixture containing similar amounts of peptides. The inhibition afforded by the mixture of the five H_{C} peptides (37.4%) was significantly higher than the sum of the inhibition values by the same amount of the individual H_{C} peptides (28.3%). Finally, the inhibition by the mixture of the 11 H_{N} and H_{C} peptides together (44.8%) was also substantially higher than the sum of inhibition values of similar amounts of the individual peptides (31.2%).

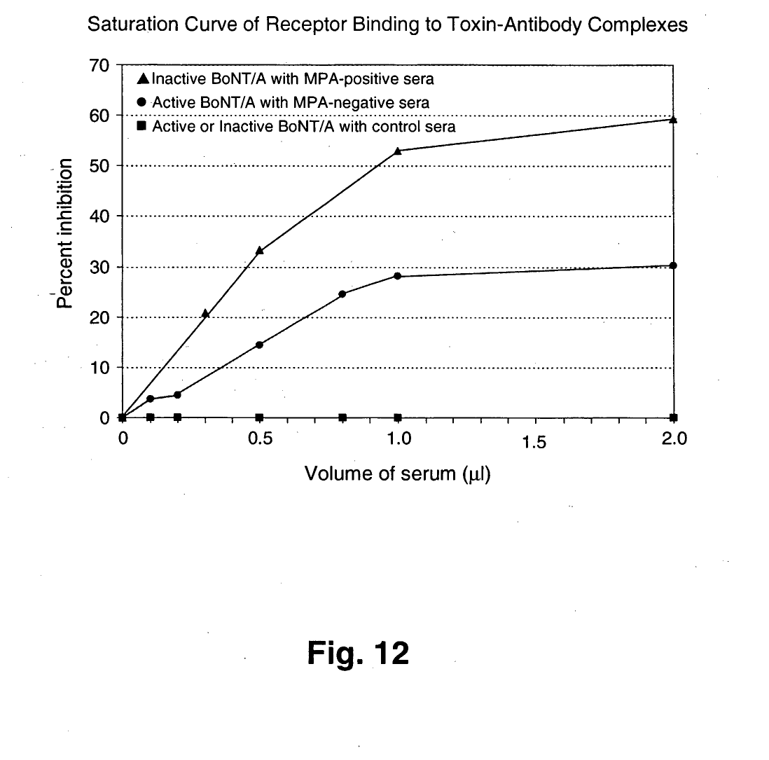

To determine the volume of a sample containing neutralizing anti-BoNT/A antibodies necessary to achieve saturation binding of a BoNT/A toxin to a BoNT/A receptor, a BoNT/A receptor binding inhibition assay was conducted. Various volumes of human serum (0 to 2 μL) and approximately 50,000 cpm of ¹²⁵Iodine-labeled active BoNT/A toxin (about 1.0 ng) were added to 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% bovine serum albumin). The reaction mixtures were incubated at 37° C. for 10 minutes in order to allow for the formation of any toxin-antibody complexes. A 4.0 μL aliquot of synaptosomes was added to the reaction mixture and incubated at 37° C. for 10 minutes in order to permit binding of any free ¹²⁵Iodine-labeled active BoNT/A toxin to BoNT/A receptor complexes on the surface of the added synaptosomes. The reaction mixture was then microcentrifuged (23,000×g at 20° C. for 3 minutes) to pellet toxin-receptor complexes. These pellets were washed twice in 800 μL of Ringer's solution, pH 7.0 to remove toxin-antibody complexes and any unbound toxin. The pellet containing toxin-receptor complexes was resuspended in 300 μL of Ringer's solution, pH 7.0, transferred to a glass scintillation tube, and the amount of radioactivity from these complexes, measured using a gamma scintillation counter. The percent BoNT/A receptor binding inhibition of a serum sample was calculated using the following formula: [1−(count of the sample/count of control)]×100. The negative control was a mixture of five serum samples taken from untreated individuals (never administered a BoNT/A therapy) and the radioactivity measured from this sample was used as a zero point reference to adjust for background. Assays were carried out in triplicates. The percent inhibition obtained with MPA positive sera that contain neutralizing antibodies reached a plateau at about 1 μL of serum, while serum from control samples, which lack neutralizing anti-BoNT/A antibodies, did not prevent the formation of toxin-receptor complexes at any volume used (see, e.g., FIG. 12).

### Example 5

### BoNT/A Immunoresistant Assay Using an Active Toxin and a Radiation Detection Method

To make a radiolabeled BoNT/A toxin, a active BoNT/A toxin is labeled with ¹²⁵Iodine using a chloramine T method as described in Example 4.

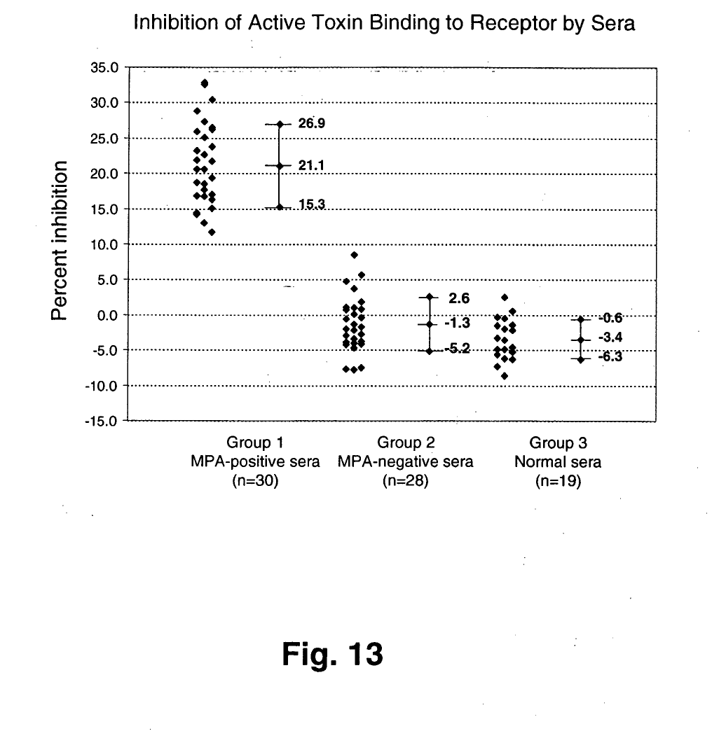

To conduct a BoNT/A Immunoresistant Assay (see, e.g., FIGS. 1 & 2), 1.0 μL of a serum sample and approximately 50,000 cpm of ¹²⁵Iodine-labeled active BoNT/A (about 1.0 ng) were added to 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% bovine serum albumin). This reaction mixture was incubated at 37° C. for 10 minutes in order to allow for the formation of any toxin-antibody complexes. A 4.0 μL aliquot of synaptosomes was added to the reaction mixture and incubated at 37° C. for 10 minutes in order to permit binding of any free ¹²⁵Iodine-labeled active BoNT/A toxin to BoNT/A receptors on the surface of the added synaptosomes. The reaction mixture was then microcentrifuged (23,000×g at 20° C. for 3 minutes) to pellet toxin-receptor complexes. These pellets were washed twice in 800 μL of Ringer's solution, pH 7.0 to remove toxin-antibody complexes and any unbound toxin. The pellet containing toxin-receptor complexes was resuspended in 300 mL of Ringer's solution, pH 7.0, transferred to a glass scintillation tube, and the amount of radioactivity from these complexes measured using a gamma scintillation counter. The percent BoNT/A receptor binding inhibition of a serum sample was calculated using the following formula: [1−(count of the sample/count of control)]×100. The negative control was a mixture of five serum samples taken from untreated individuals (never administered a BoNT/A therapy) and the radioactivity measured from this sample was used as a zero point reference to adjust for background. Assays were carried out in triplicates. As shown in FIG. 13, sera obtained from Group 1 had statistically significant higher inhibitory activities (mean=21.1±5.8) relative to sera obtained from Group 2 (mean=−1.3±3.9; p<0.0001) or control sera (mean=−3.4±2.8; p<0.0001). These data indicating that that the BoNT/A Immunoresistant Assay using active BoNT/A toxin can distinguish between neutralizing and non-neutralizing anti-BoNT/A antibodies present in sera collected from cervical dystonia patients that were treated with BoNT/A toxin.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 6

### BoNT/A Immunoresistant Assay Using an Inactive Toxin and a Radiation Detection Method

To make radioactively labeled inactive BoNT/A toxin, formaldehyde-inactivated BoNT/A (Metabiologics, Inc., Madison, Wis.) was dialysis in Spectrapor membrane with 3,500 molecular weight cut-off (Spectrum Medical Industries, Los Angeles, Calif.) against a solution comprising 100 mM phosphate-buffered saline, pH 7.4 and 50 mM sodium chloride to remove the formaldehyde. Glycerin was added to the dialysized inactive toxin to a final concentration of 25% (v/v) and stored at −20° C. A 1.0 μg sample of dialysized BoNT/A toxin was labeled with ¹²⁵Iodine using a chloramine T method as described in Example 4, except 1.0 μg of dialysized inactive BoNT/A toxin was added to the labeling reaction instead of 1.0 μg of active BoNT/A toxin. The ¹²⁵Iodine-labeled inactive BoNT/A toxin was stored at 4° C. and used within two days.

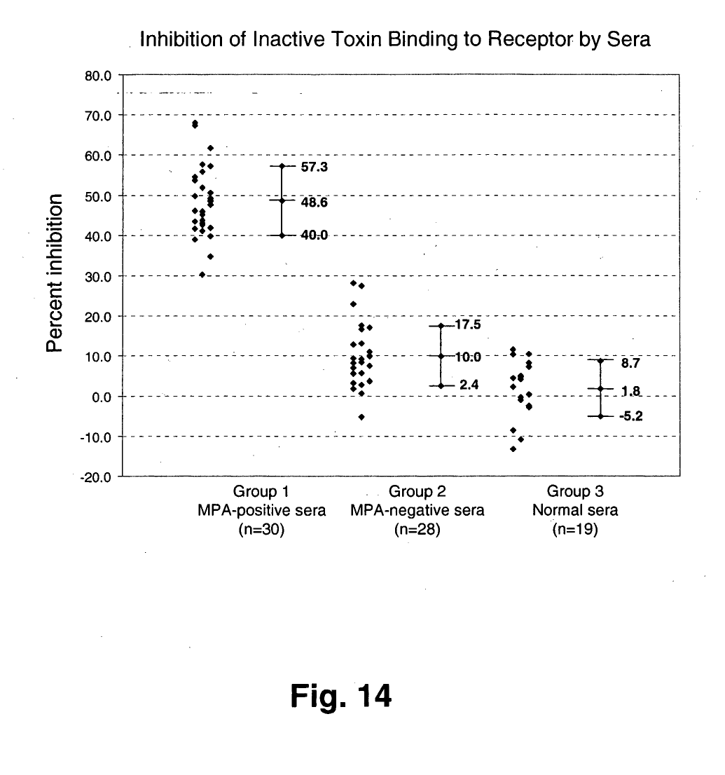

A BoNT/A Immunoresistant Assay was performed as described in Example 5, except that 50,000 cpm of ¹²⁵Iodine-labeled inactive BoNT/A toxin (about 1 ng) was used instead of 50,000 cpm of ¹²⁵Iodine-labeled active BoNT/A toxin (about 1 ng). As shown in FIG. 14, sera obtained from Group 1 had distinctly higher inhibitory activities (mean=48.6±8.7) relative to sera obtained from Group 2 (mean=−10.0±7.6; p<0.0001) or control sera (mean=1.8±6.9; p<0.0001). These data indicating that that the BoNT/A Immunoresistant Assay using inactive BoNT/A toxin can distinguish between neutralizing and non-neutralizing anti-BoNT/A antibodies present in sera collected from cervical dystonia patients that were treated with BoNT/A.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 7

### BoNT/A Immunoresistant Assay Using an Inactive his-BoNT/A (H227Y) and a Fluorescence Detection Method

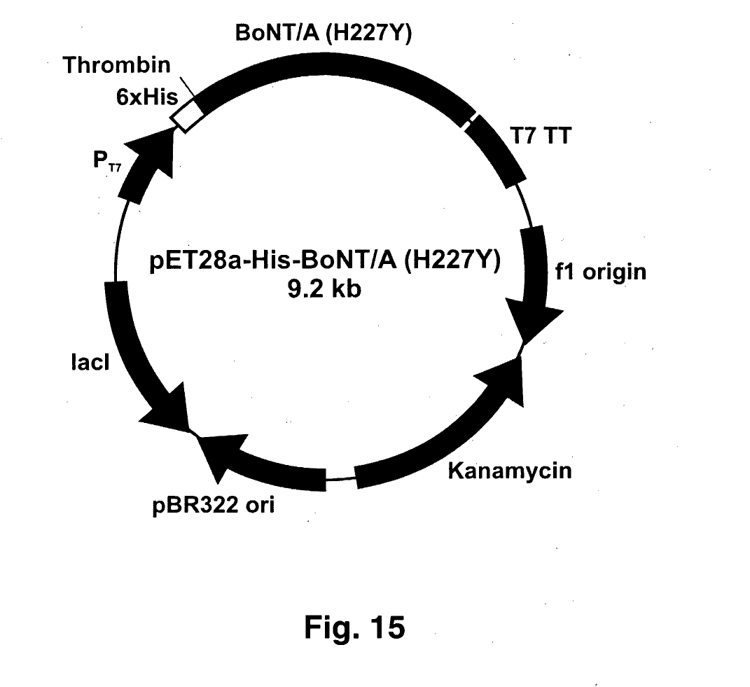

To express a BoNT/A toxin, a pET28aHis-BoNT/A (H227Y) expression construct (FIG. 15) comprising a BoNT/A (H227Y) nucleic acid molecule of SEQ ID NO: 4 is introduced into chemically competent E. coli BL21 (DE3) cells (Invitrogen, Inc, Carlsbad, Calif.) using a heat-shock transformation protocol. The heat-shock reaction is plated onto 1.5% Luria-Bertani agar plates (pH 7.0) containing 50 μg/mL of Kanamycin and placed in a 37° C. incubator for overnight growth. A Kanamycin-resistant colony of transformed E. coli containing the pET28a-His-BoNT/A (H227Y) construct is used to inoculate a 15 mL tube containing 3.0 mL of Luria-Bertani media containing 50 μg/mL of Kanamycin that is then placed in a 37° C. incubator, shaking at 250 rpm, for overnight growth. Approximately 600 μL of the resulting overnight starter culture is used to inoculate a 3.0 L baffled flask containing 600 mL of Overnight Express™ autoinduction medium (EMD Biosciences-Novagen, Madison, Wis.) containing 50 μg/mL of Kanamycin at a dilution of 1:1000. The inoculated culture is grown in a 37° C. incubator shaking at 250 rpm for approximately 5.5 hours until mid-log phase is reached (OD₆₀₀ of about 0.6-0.8) and the culture is then transferred to a 16° C. incubator shaking at 250 rpm for overnight expression. Cells are harvested by centrifugation (4,000 rpm at 4° C. for 20-30 minutes).

To purify a BoNT/A toxin, the bacterial cell pellet harvested above is resuspended in Column Binding Buffer (25 mM N-(2-hydroxyethyl) propanesulfonic acid (HEPES), pH 7.8; 500 mM sodium chloride; 10 mM imidazole; 2× Protease Inhibitor Cocktail Set III (EMD Biosciences-Calbiochem, San Diego Calif.); 5 units/mL of Benzonase (EMD Biosciences-Novagen, Madison, Wis.); 0.1% (v/v) Triton-X® 100 (4-octylphenol polyethoxylate); 10% (v/v) glycerol), and then transferred to a cold Oakridge centrifuge tube. The cell suspension is sonicated on ice (10-12 pulses of 10 seconds at 40% amplitude with 60 seconds cooling intervals on a Branson Digital Sonifier) in order to lysis the cells and release the His-BoNT/A (H227Y) peptide, and then centrifuged (16,000 rpm at 4° C. for 20 minutes) to clarify the lysate. An immobilized metal affinity chromatography (IMAC) column is prepared using a 20 mL Econo-Pac column support (Bio-Rad Laboratories, Hercules, Calif.) packed with 2.5-5.0 mL of TALON™ SuperFlow Co²⁺ affinity resin (BD Biosciences-Clontech, Palo Alto, Calif.), which is then equilibrated by rinsing with 5 column volumes of deionized, distilled water, followed by 5 column volumes of Column Binding Buffer. The clarified lysate is applied slowly to the equilibrated column by gravity flow (approximately 0.25-0.3 muminute). The column is then washed with 5 column volumes of Column Wash Buffer (25 mM N-(2-hydroxyethyl) propanesulfonic acid (HEPES), pH 7.8; 500 mM sodium chloride; 10 mM imidazole; 0.1% (v/v) Triton-X® 100 (4-octylphenol polyethoxylate); 10% (v/v) glycerol). The His-BoNT/A (H227Y) peptide is eluted with 20-30 mL of Column Elution Buffer (25 mM N-(2-hydroxyethyl) propanesulfonic acid (HEPES), pH 7.8; 500 mM sodium chloride; 500 mM imidazole; 0.1% (v/v) Triton-X® 100 (4-octylphenol polyethoxylate); 10% (v/v) glycerol) and collected in approximately twelve 1 mL fractions. The amount of His-BoNT/A (H227Y) peptide contained in each elution fraction is determined by a Bradford dye assay. In this procedure, 20 μL aliquot from each 1.0 mL fraction is combined with 200 μL of Bio-Rad Protein Reagent (Bio-Rad Laboratories, Hercules, Calif.), diluted 1 to 4 with deionized, distilled water, and then the intensity of the colorimetric signal is measured using a spectrophotometer. The five fractions with the strongest signal are considered to comprise the elution peak and are pooled. Total protein yield are determined by estimating the total protein concentration of the pooled peak elution fractions using bovine gamma globulin as a standard (Bio-Rad Laboratories, Hercules, Calif.). The amount of His-BoNT/A (H227Y) peptide is adjusted to a protein concentration suitable for a fluorescence labeling reaction.

To make a BoNT/A toxin operably-linked to a fluorescence compound, the purified His-BoNT/A (H227Y) peptide described above is labeled with an using a Alexa Fluor® 488 Protein Labeling Kit (Molecular Probes, Inc., Eugene, Oreg.). A labeling reaction comprising 500 μL of 2 μg/μL of His-BoNT/A (H227Y) peptide and 50 μL of 100 mM sodium carbonate is added to a vial containing the Alexa Fluor® 488 dye. This reaction mixture is incubated at room temperature with stirring for 60 minutes to allow the dye to dissolve and become attached to the peptide. Excess Alexa Fluor® 488 dye is removed from the Alexa Fluor® 488-labeled toxin by applying the labeling mixture through a Bio-Gel P30 Fine (Bio-Rad Laboratories, Hercules, Calif.) size exclusion column equilibrated and eluted with a column solution comprising 10 mM potassium phosphate, pH 7.2; 150 mM sodium chloride; 0.1% bovine serum albumin (BSA); and 0.01% sodium azide). The His-BoNT/A (H227Y) peptide is eluted with 1.0 mL of column solution and collected in ten 100 μL fractions. The progress of the Alexa Fluor® 488-labeled His-BoNT/A (H227Y) peptide sample through the column as well as which elution fractions contain the sample is monitored using an ultraviolet light from a hand-held transilluminator. The amount of Alexa Fluor® 488 incorporation and protein concentration is determined for each fraction by measuring the absorbance of each 100 μL fraction at 280 nm and 494 nm using a SpectraMax Gemini XPS spectrofluorometer (Molecular Devices Corp., Sunnyvale, Calif.). Fractions containing eluted Alexa Fluor® 488-labeled His-BoNT/A (H227Y) peptide are pooled together, adjusted to a protein concentration suitable for the BoNT/A Immunoresistant Assay, and stored at 4° C. in a light-tight container.

To conduct a BoNT/A Immunoresistant Assay (see, e.g., FIGS. 1 & 2), 1.0 μL of a serum sample and approximately 1 ng of Alexa Fluor® 488-labeled His-BoNT/A (H227Y) peptide are added to 100 μL of Ringer's solution, pH 7.0. This reaction mixture is incubated at 37° C. for 10 minutes in order to allow for the formation of any toxin-antibody complexes. A 4.0 μL aliquot of synaptosomes is added to the reaction mixture and incubated at 37° C. for 10 minutes in order to permit binding of any free Alexa Fluor® 488-labeled His-BoNT/A (H227Y) to BoNT/A receptors on the surface of the added synaptosomes. The reaction mixture is then microcentrifuged (23,000×g at 20° C. for 3 minutes) to pellet toxin-receptor complexes. These pellets are washed twice in 800 μL of Ringer's solution, pH 7.0 to remove toxin-antibody complexes and any unbound Alexa Fluor® 488-labeled His-BoNT/A (H227Y) peptide. The pellet containing toxin-receptor complexes is resuspended in 300 μL of Ringer's solution, pH 7.0. The fluorescence of a 100 μL aliquoit of the resuspended toxin-receptor complex pellet is measured by determining the absorbance of the sample at 280 nm and 494 nm using a SpectraMax Gemini XPS spectrofluorometer (Molecular Devices Corp., Sunnyvale Calif.). The percent synaptosome binding inhibition of a serum sample is calculated using the following formula: [1−(fluorescence/fluorescence of control)]×100. The negative control is a mixture of five serum samples taken from untreated individuals (never before administered a BoNT/A therapy) and the fluorescence measured from this sample is used as a zero point reference to adjust for background. Assays are carried out in triplicates.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 8

### Construction of pQB167-H_{C}BoNT/A-GFP and Expression of a BoNT/A Toxin Using a Prokaryotic Expression System

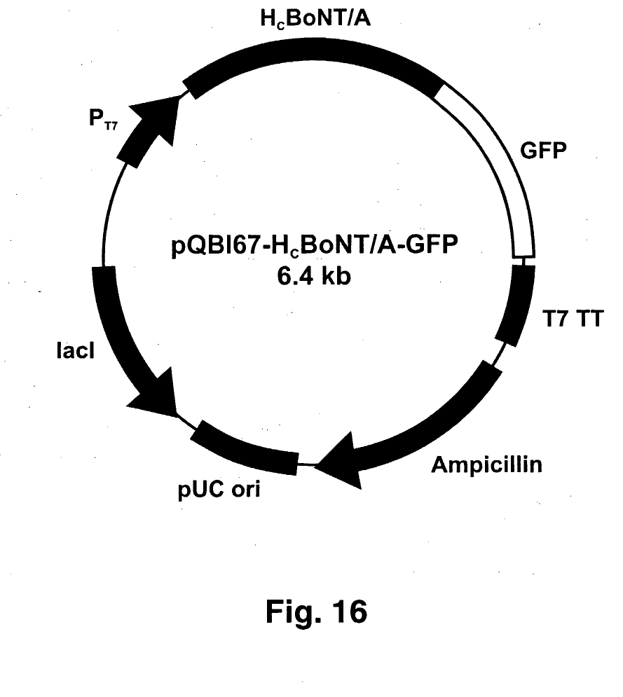

To make a BoNT/A toxin operably-linked to a photoprotein, a nucleic acid fragment encoding the amino acid region Arginine 861 through Leucine 1296 of the BoNT/A H_{C} domain of SEQ ID NO: 8 is amplified from Clostridium botulinum strain 62A genomic DNA using a polymerase chain reaction method and subcloned into a pCR2.1 vector using the TOPO® TA cloning method (Invitrogen, Inc, Carlsbad, Calif.). The forward and reverse oligonucleotide primers used for this reaction are designed to include unique restriction enzyme sites useful for subsequent subcloning steps. The resulting pCR2.1-H_{C}BoNT/A construct is digested with restriction enzymes that 1) excise the insert containing the entire open reading frame encoding the H_{C} domain of BoNT/A; and 2) enable this insert to be operably-linked to a pQB167 vector (Qbiogene, Inc., Carlsbad, Calif.). This insert is subcloned using a T4 DNA ligase procedure into a pQB167 vector that is digested with appropriate restriction endonucleases to yield pQB167-H_{C}BoNT/A-GFP (FIG. 16). The ligation mixture is transformed into chemically competent E. coli BL21 (DE3) cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the inset. This cloning strategy yields a prokaryotic expression construct encoding the H_{C} domain of BoNT/A operably-linked to carboxyl-terminal Green Fluorescent Protein (FIG. 16).

To express a BoNT/A-GFP using bacteria, a pQB167-H_{C}BoNT/A-GFP expression construct is introduced into chemically competent E. coli BL21 (DE3) cells (Invitrogen, Inc, Carlsbad, Calif.) using a heat-shock transformation protocol. The heat-shock reaction is then plated onto 1.5% Luria-Bertani agar plates (pH 7.0) containing 100 μg/mL of Ampicillin and placed in a 37° C. incubator for overnight growth. A single Ampicillin-resistant colony of transformed E. coli containing pQB167-H_{C}BoNT/A-GFP is used to inoculate a 15 mL test tube containing 3.0 mL Luria-Bertani media, (pH 7.0) containing 100 μg/mL of Ampicillin which is then placed in a 37° C. incubator, shaking at 250 rpm, for overnight growth. The resulting overnight starter culture is used to inoculate a 1.0 L baffled flask containing 100 mL Luria-Bertani media, (pH 7.0) containing 100 μg/mL of Ampicillin at a dilution of 1:1000. This culture is grown in a 32° C. incubator shaking at 250 rpm for approximately 6.5 hours until mid-log phase is reached (OD₆₀₀ Of about 0.6-0.8). Protein expression is then induced by adding 1 mM isopropyl-β-D-thiogalactopyranoside (IPTG) and the culture is placed in a 32° C. incubator shaking at 250 rpm for overnight expression. Cells are harvested by centrifugation (4,000 rpm at 4° C. for 20-30 minutes) and used immediately, or stored dry at −80° C. until needed.

### Example 9

### Construction of pQBI 25-H_{C}BoNT/A-GFP and Expression of a BoNT/A Toxin using a Mammalian Expression System

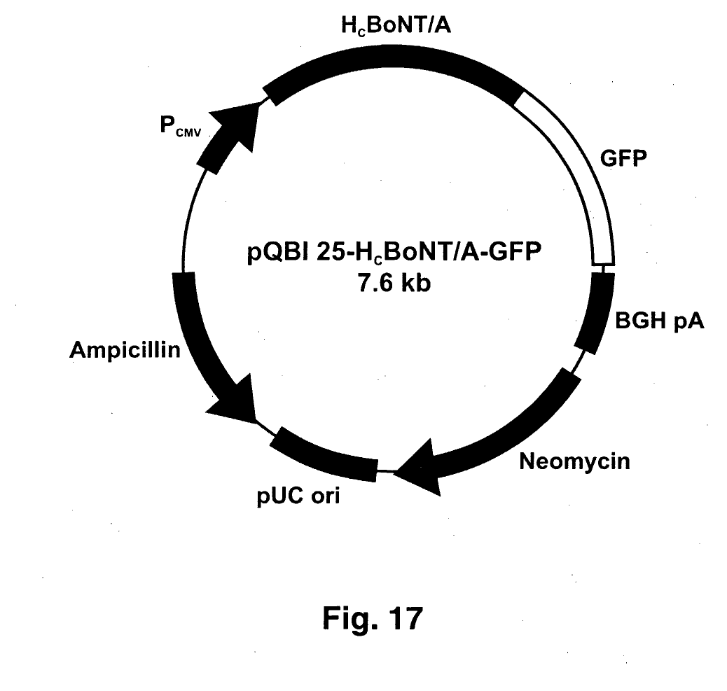

To make a BoNT/A toxin operably-linked to a florescence peptide, a nucleic acid fragment encoding the amino acid region Arginine 861 through Leucine 1296 of the H_{C} domain of BoNT/A is amplified from Clostridium botulinum strain 62A genomic DNA using a polymerase chain reaction method and subcloned into a pCR2.1 vector using the TOPOE TA cloning method (Invitrogen, Inc, Carlsbad, Calif.). The forward and reverse oligonucleotide primers used for this reaction are designed to include unique restriction enzyme sites useful for subsequent subcloning steps. The resulting pCR2.1-H_{C}BoNT/A construct is digested with restriction enzymes that 1) excise the insert containing the entire open reading frame encoding the H_{C} domain of BoNT/A; and 2) enable this insert to be operably-linked to a pQBI 25 vector (Qbiogene, Inc., Carlsbad, Calif.). This insert is subcloned using a T4 DNA ligase procedure into a pQBI 25 vector that is digested with appropriate restriction endonucleases to yield PQBI 25-H_{C}BoNT/A-GFP (FIG. 17). The ligation mixture is transformed into chemically competent E. coli BL21 (DE3) cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the inset. This cloning strategy yields a mammalian expression construct encoding the H_{C} domain of BoNT/A operably-linked to carboxyl-terminal GFP peptide (FIG. 17).

To express a BoNT/A toxin using a mammalian cell line, about 1.5×10⁵. SH-SY5Y cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen, Inc, Carlsbad, Calif.) and 1×MEM non-essential amino acids solution (MEM) non-essential amino acids solution (Invitrogen, Inc, Carlsbad, Calif.), and grown in a 37° C. incubator under 5% carbon dioxide until the cells reach a density of about 5×10⁵ cells/ml (6-16 hours). A 600 μL transfection solution is prepared by adding 300 μL of 2×HEPES-Buffered Saline, pH 7.1 (50 mM N-(2-hydroxyethyl) propanesulfonic acid (HEPES), pH 7.4; 1.5 mM sodium phosphate (monobasic); 280 mM sodium chloride) to 300 μL of 240 mM of calcium chloride containing 19 μg of pQBI 25-H_{C}BoNT/A-GFP and this solution is incubated for approximately 30 minutes. The transfection solution is added to the SH-SY5Y cells and the cells are incubated in a 37° C. incubator under 5% carbon dioxide for 16-24 hours. Transfection media is replaced with 3 mL of fresh complete, supplemented DMEM and cells are grown in a 37° C. incubator under 5% carbon dioxide for 48 hours. Cells are harvest by rinsing cells once with 3.0 mL of 100 mM phosphate-buffered saline, pH 7.4 and detaching rinsed cells by adding 500 μl of 100 mM phosphate-buffered saline, pH 7.4 and scraping cells from the culture plate. Detached cells are transferred to a 1.5 mL test tube and pelleted by microcentrifugation (10,000×g at 4° C. for 5 minutes). The supernatant is discarded and the cell pellet is used immediately for subsequent steps, or stored at −80° C. until needed.

### Example 10

### BoNT/A Immunoresistant Assay Using a H_{C}BoNT/A-GFP and a Fluorescence Detection Method

To purify a GFP-linked BoNT/A toxin, a cell pellet expressing a pQBI 25-H_{C}BoNT/A-GFP construct is resuspended in 10 mL of Tris-EDTA Buffer (10 mM Tris-HCl, pH 8.0; 1 mM EDTA, pH 8.0), containing 1 mg/mL of lysozyme and the cells are lysed using three freeze-thaw rounds consisting of −80° C. for 5 minutes then 37° C. for 5 minutes. The cell lysate is centrifuged (5,000×g at 4° C. for 15 minutes) to pellet the cellular debris and the supernatant is transferred to a new tube containing an equal volume of Column Binding Buffer (4 M ammonium sulfate). A hydrophobic interaction chromatography (HIC) column is prepared using a 20 mL Econo-Pac column support (Bio-Rad Laboratories, Hercules, Calif.) that is packed with 2.5-5.0 mL of methyl HIC resin (Bio-Rad Laboratories, Hercules, Calif.), which is then equilibrated by rinsing with 5 column volumes of Column Equilibration Buffer (2 M ammonium sulfate). The clarified lysate is applied slowly to the equilibrated column by gravity flow (approximately 0.25-0.3 muminute). The column is then washed with 5 column volumes of Column Wash Buffer (1.3 M ammonium sulfate). The H_{C}BoNT/A-GFP peptide is eluted with 20-30 mL of Column Elution Buffer (10 mM TE Buffer) and is collected in approximately twelve 1 mL fractions. The progress of the H_{C}BoNT/A-GFP peptide sample through the column as well as which elution fractions contain the sample is monitored using an ultraviolet light from a hand-held transilluminator. The amount of H_{C}BoNT/A-GFP peptide contained in each elution fraction is determined by a Bradford dye assay. In this procedure, 20 μL aliquot from each 1.0 mL fraction is combined with 200 μL of Bio-Rad Protein Reagent (Bio-Rad Laboratories, Hercules, Calif.), diluted 1 to 4 with deionized, distilled water, and then the intensity of the colorimetric signal is measured using a spectrophotometer. The five fractions with the strongest signal are considered to comprise the elution peak and are pooled. Total protein yield are determined by estimating the total protein concentration of the pooled peak elution fractions using bovine gamma globulin as a standard (Bio-Rad Laboratories, Hercules, Calif.). The amount of H_{C}BoNT/A-GFP is adjusted to a protein concentration suitable for the BoNT/A Immunoresistant Assay.

To conduct a BoNT/A Immunoresistant Assay (see, e.g., FIGS. 1 & 2), 1.0 μL of a serum sample and approximately 1 ng of H_{C}BoNT/A-GFP are added to 100 μL of Ringer's solution, pH 7.0. This reaction mixture is incubated at 37° C. for 10 minutes in order to allow for the formation of any toxin-antibody complexes. A 4.0 μL aliquot of synaptosomes is added to the reaction mixture and incubated at 37° C. for 10 minutes in order to permit binding of any free H_{C}BoNT/A-GFP to BoNT/A receptors on the surface of the added synaptosomes. The reaction mixture is then microcentrifuged (23,000×g at 20° C. for 3 minutes) to pellet toxin-receptor complexes. These pellets are washed twice in 800 μL of Ringer's solution, pH 7.0 to remove toxin-antibody complexes and any unbound H_{C}BoNT/A-GFP. The pellet containing toxin-receptor complexes is resuspended in 300 μL of Ringer's solution, pH 7.0. The fluorescence of the resuspended pellet is measured at 280 nm, 474 nm and 509 nm using a SpectraMax Gemini XPS spectrofluorometer (Molecular Devices Corp., Sunnyvale Calif.). The percent BoNT/A receptor binding inhibition of a serum sample is calculated using the following formula: [1−(fluorescence of the sample/fluorescence of control)]×100. The negative control is a mixture of five serum samples taken from untreated individuals (never before administered a BoNT/A therapy) and the fluorescence measured from this sample is used as a zero point reference to adjust for background. Assays are carried out in triplicates.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 11

### BoNT/A Immunoresistant Assay Using an Inactive his-BoNT/A (H227Y) Toxin and a Chemiluminescence Detection Method

Expression of a BoNT/A (H227Y) is performed as described in Example 7.

Purification of a BoNT/A toxin using an immobilized metal affinity chromatography column is performed as described in Example 7, except amount of His-BoNT/A (H227Y) is adjusted to a protein concentration suitable for a chemiluminescence labeling reaction.

To make a BoNT/A toxin operably-linked to a chemiluminescence compound, the purified His-BoNT/A (H227Y) described above is labeled with an acridinium ester using a chemiluminescence labeling kit (Cayman Chemical Co., Ann Arbor, Mich.). A labeling reaction comprising 300 μl of Acridinium Labeling Buffer (100 mM phosphate buffer, pH 8.0; 150 mM sodium chloride, containing 50 μg of His-BoNT/A (H227Y) peptide) and 10 μl of 0.5 mM acridinium ester in dimethyl formamide is incubated with rotation at room temperature for 30 minutes. To this labeling reaction, 100 μl of Acridinium Quenching Solution (100 mM phosphate buffer, pH 8.0; 150 mM sodium chloride; 1% lycine) is added and allowed to incubate with rotation at room temperature for 30 minutes to stop the reaction. Excess acridinium ester is removed from the acridinium-labeled toxin by applying the labeling mixture through a size exclusion column (6,000 MW cut-off) equilibrated with 20 volumes of Acridinium Purification Buffer (100 mM phosphate buffer, pH 6.3; 150 mM sodium chloride, 0.1% (w/v) bovine serum albumin; 0.01% sodium azide). The His-BoNT/A (H227Y) peptide is eluted with 10 mL of Column Elution Buffer and collected in ten 1 mL fractions. The amount of acridinium incorporation and protein concentration is determined for each fraction by measuring the absorbance of a 100 μL aliquoit of the fraction at 280 nm and 367 nm using a SpectraMax Gemini XPS spectrofluorometer (Molecular Devices Corp., Sunnyvale Calif.). Fractions containing eluted acridinium-labeled His-BoNT/A (H227Y) peptide are pooled together, adjusted to a protein concentration suitable for the BoNT/A Immunoresistant Assay, and stored at −20° C.

To conduct a BoNT/A Immunoresistant Assay (see, e.g., FIGS. 1 & 2), 1.0 μL of a serum sample and approximately 1 ng of acridinium-labeled His-BoNT/A (H227Y) are added to 100 μL of Ringer's solution, pH 7.0. This reaction mixture is incubated at 37° C. for 10 minutes in order to allow for the formation of any toxin-antibody complexes. A 4.0 μL aliquot of synaptosomes is added to the reaction mixture and incubated at 37° C. for 10 minutes in order to permit binding of any free acridinium-labeled His-BoNT/A (H227Y) to BoNT/A receptors on the surface of the added synaptosomes. The reaction mixture is then microcentrifuged (23,000×g at 20° C. for 3 minutes) to pellet toxin-receptor complexes. These pellets are washed twice in 800 μL of Ringer's solution, pH 7.0 to remove toxin-antibody complexes and any unbound acridinium-labeled His-BoNT/A (H227Y) peptide. The pellet containing toxin-receptor complexes is resuspended in 300 μL of Ringer's solution, pH 7.0. The chemiluminescence of the resuspended toxin-receptor complex pellet is determined by measuring the luminescence of a 10 μL aliquoit of the resuspended complex using a TD-20/20 Luminometer (Turner BioSystems, Inc., Sunnyvale Calif.), after the sequential autoinjection of 50 μL 200 mM sodium hydroxide and 50 μL of 0.06% hydrogen peroxide. The percent BoNT/A receptor binding inhibition of a serum sample is calculated using the following formula: [1−(relative light unit of the sample/relative light unit of control)]×100. The negative control is a mixture of five serum samples taken from untreated individuals (never before administered a BoNT/A therapy) and the chemiluminescence measured from this sample is used as a zero point reference to adjust for background. Assays are carried out in triplicates.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 12

### BoNT/A Immunoresistant Assay Using an BoNT/A Fragment and a Bioluminescence Detection Method

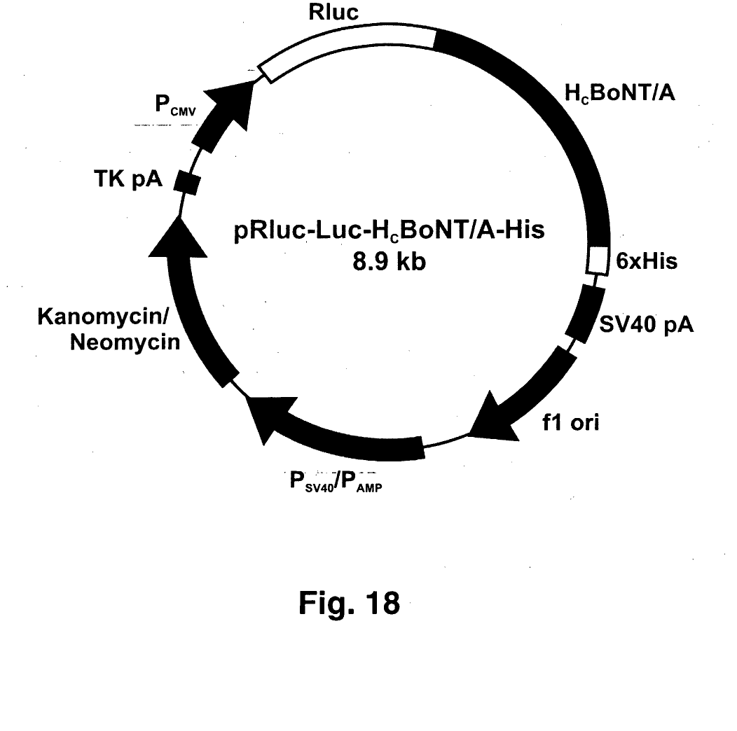

To make a BoNT/A fragment operably-linked to a bioluminescence peptide, a nucleic acid fragment encoding the amino acid region Arginine 861 through Leucine 1296 of the H_{C} domain of BoNT/A is amplified from Clostridium botulinum strain 62A genomic DNA using a polymerase chain reaction method and subcloned into a pCR2.1 vector using the TOPO® TA cloning method (Invitrogen, Inc, Carlsbad, Calif.). The forward and reverse oligonucleotide primers used for this reaction are designed to include unique restriction enzyme sites useful for subsequent subcloning steps. In addition, the reverse primer also includes the nucleic acid composition of SEQ ID NO: 10, encoding a polyhistidine peptide of SEQ ID NO: 9. The resulting pCR2.1-H_{C}BoNT/A construct is digested with restriction enzymes that 1) excise the insert containing the entire open reading frame encoding the H_{C} domain of BoNT/A; and 2) enable this insert to be operably-linked to a pRluc-C1 vector (BioSignal Packard, Montréal, Québec, Canada). This insert is subcloned using a T4 DNA ligase procedure into a pQB167 vector that is digested with appropriate restriction endonucleases to yield pRIucC-Luc-H_{C}BoNT/A-His (FIG. 18). The ligation mixture is transformed into chemically competent E. coli BL21 (DE3) cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the inset. This cloning strategy yields a mammalian expression construct encoding the BoNT/A H_{C} domain of SEQ ID NO: 7 operably-linked to an amino-terminal luciferase peptide and a carboxyl-terminal poly histidine peptide (FIG. 18).

Expression of a BoNT/A toxin in a mammalian cell line is performed as described in Example 9, except the expression construct pRIucC-Luc-H_{C}BoNT/A-His is added to the transfection mixture instead of pQBI 25-H_{C}BoNT/A-GFP.

Purification of a BoNT/A toxin using an immobilized metal affinity chromatography column is performed as described in Example 7, except the Luc-H_{C}BoNT/A-His is purified instead of the BoNT/A-His (H227Y). The amount of Luc-H_{C}BoNT/A-His is adjusted to a protein concentration suitable for the BoNT/A Immunoresistant Assay.

To conduct a BoNT/A Immunoresistant Assay (see, e.g., FIGS. 1 & 2), 1.0 μL of a serum sample and approximately 1 ng of Luc-H_{C}BoNT/A-His peptide are added to 100 μL of Ringer's solution, pH 7.0. This reaction mixture is incubated at 37° C. for 10 minutes in order to allow for the formation of any toxin-antibody complexes. A 4.0 μL aliquot of synaptosomes is added to the reaction mixture and incubated at 37° C. for 10 minutes in order to permit binding of any free Luc-H_{C}BoNT/A-His to BoNT/A receptors on the surface of the added synaptosomes. The reaction mixture is then microcentrifuged (23,000×g at 20° C. for 3 minutes) to pellet toxin-receptor complexes. These pellets are washed twice in 800 μL of Ringer's solution, pH 7.0 to remove toxin-antibody complexes and any unbound H_{C}-GFP. The pellet containing toxin-receptor complexes is resuspended in 300 μL of Ringer's solution, pH 7.0. The bioluminescence of the resuspended toxin-receptor complex pellet is determined by measuring the luminescence of a 10 μL aliquot of the resuspended complex using the Luciferase Assay System (Promega Corp., Madison, Wis.) and a TD-20/20 Luminometer (Turner BioSystems, Inc., Sunnyvale Calif.). The percent synaptosome binding inhibition of a serum sample is calculated using the following formula: [1−(relative light unit of the sample/relative light unit of control)]×100. The negative control is a mixture of five serum samples taken from untreated individuals (never before administered a BoNT/A therapy) and the fluorescence measured from this sample is used as a zero point reference to adjust for background. Assays are carried out in triplicates.

In an alternative procedure to the BoNT/A Immunoresistant Assay described above, approximately 1 ng of an FGFR3 disclosed in the present specification can be substituted for the 4.0 μL aliquot of synaptosomes. In this procedure a FGFR3 is recombinantly expressed and purified as described in Example 13 and the amount of FGFR3 is adjusted to a protein concentration suitable for a BoNT/A Immunoresistant Assay.

### Example 13

### Construction and Expression of Constructs Expressing an FGFR3

To construct pET28/His-FGFR3, a nucleic acid fragment encoding the amino acid region comprising FGFR3 of SEQ ID NO: 12 is amplified from a human brain cDNA library using a polymerase chain reaction method and subcloned into a pCR2.1 vector using the TOPO® TA cloning method (Invitrogen, Inc, Carlsbad, Calif.). The forward and reverse oligonucleotide primers used for this reaction are designed to include unique restriction enzyme sites useful for subsequent subcloning steps. The resulting pCR2.1/FGFR3 construct is digested with restriction enzymes that 1) excise the insert containing the entire open reading frame encoding the FGFR3; and 2) enable this insert to be operably-linked to a pET28 vector (EMD Biosciences-Novagen, Madison, Wis.). This insert is subcloned using a T4 DNA ligase procedure into a pET28a vector that is digested with appropriate restriction endonucleases to yield pET28/His-FGFR3. The ligation mixture is transformed into chemically competent E. coli BL21 (DE3) cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the inset. This cloning strategy yielded a prokaryotic expression construct comprising the nucleic acid molecule of SEQ ID NO: 12 encoding the FGFR3 of SEQ ID NO: 11 operably-linked to an amino terminal polyhistidine affinity binding peptide of SEQ ID NO: 9. A similar cloning strategy is used to make prokaryotic expression constructs containing any one of the nucleic acid molecules of SEQ ID NO: 12, 14, 16, 18, 20, 22, 24, 26, 28, 30, 32, 34, 36 and 38, encoding the FGFR3s of SEQ ID NO: 11, 13, 15, 17, 19, 21, 23, 25, 27, 29, 31, 33, 35 and 37, respectively, operably linked to a pET28 vector.

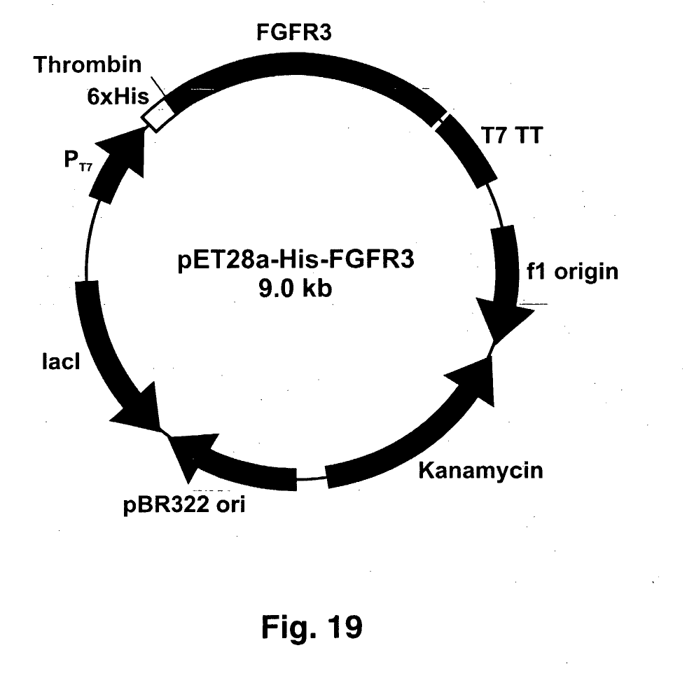

To express a FGFR3, a pET28/His-FGFR3 expression construct (FIG. 19) comprising a FGFR3 nucleic acid composition of SEQ ID NO: 12 is introduced into chemically competent E. coli BL21 (DE3) cells (Invitrogen, Inc, Carlsbad, Calif.) using a heat-shock transformation protocol. The heat-shock reaction is plated onto 1.5% Luria-Bertani agar plates (pH 7.0) containing 50 μg/mL of Kanamycin and placed in a 37° C. incubator for overnight growth. Kanamycin-resistant colonies of transformed E. coli containing pET28/His-FGFR3 are used to inoculate baffled flask containing 3.0 mL of PA-0.5G media containing 50 μg/mL of Kanamycin which are then placed in a 37° C. incubator, shaking at 250 rpm, for overnight growth. The resulting overnight starter culture are in turn used to inoculate a 3 L baffled flask containing 600 mL of ZYP-5052 autoinducing media and 50 μg/mL of Kanamycin at a dilution of 1:1000. The inoculated culture is grown in a 37° C. incubator shaking at 250 rpm for approximately 5.5 hours until mid-log phase is reached (OD₆₀₀ of about 0.6-0.8). Cultures are then transferred to a 16° C. incubator shaking at 250 rpm for overnight expression. Cells were harvested by centrifugation (4,000 rpm at 4° C. for 20-30 minutes) and used immediately, or stored dry at −80° C. until needed. To purify a FGFR3, an IMAC protein purification procedure described in Example 7 is used. The amount of FGFR3 is adjusted to a protein concentration suitable for the desired method.

### Example 14

### BoNT/A Immunoresistant Assay Using a FGFR3, a BoNT/A Support and a Radioactive Detection Method

To express and purify a FGFR3, an expression procedure and an IMAC protein purification procedure as described in Example 13 is used. The amount of FGFR3 is adjusted to a protein concentration suitable for a radioisotope labeling reaction.

To make a radiolabeled FGFR3, a FGFR3 is labeled with ¹²⁵Iodine using a chloramine T method as described in Example 4, except a FGFR3 is substituted for a BoNT/A toxin.

To make a BoNT/A toxin support comprising a solid or insoluble material, 50 μL of Phosphate Buffered Saline, pH 7.2 (10 mM sodium phosphate, pH 7.2; 150 mM sodium chloride) containing approximately 1 μg inactive BoNT/A toxin (see, e.g., Example 6) is added to the wells of flexible polyvinyl chloride 96-well microplates (BD Biosciences, San Jose, Calif.) and allowed to bind at 4° C. for 18 hours. The plates are then washed five times with Phosphate Buffered Saline, pH 7.2 and then blocked with 1% (w/v) bovine serum albumin at 37° C. for 60 minutes. The plates are then washed five times with Phosphate Buffered Saline, pH 7.2 containing 0.1% (w/v) bovine serum albumin and stored in this solution at 4° C. until needed.

To determine the amount of FGFR3 required to achieve saturation binding with a fixed amount of BoNT/A toxin, a receptor binding assay is conducted as described in Example 4 and as modified below, except that the receptor component is a ¹²⁵-labeled FGFR3 and the BoNT/A toxin component is unlabeled and attached to a solid or insoluble material.

To determine the volume of a sample containing neutralizing anti-BoNT/A antibodies necessary to achieve saturation binding of a BoNT/A toxin to a receptor, a receptor binding inhibition assay is conducted as described in Example 4 and as modified below, except that the receptor component is a ¹²⁵-labeled FGFR3 and the BoNT/A toxin component is unlabeled and attached to a solid or insoluble material.

To conduct a BoNT/A Immunoresistant Assay (see, e.g., FIG. 3), an optimally determined volume of a serum sample added to 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% bovine serum albumin) is applied to a BoNT/A toxin support comprising a solid or insoluble material. This reaction mixture is incubated at 37° C. for 20 minutes in order to allow for the formation of any toxin-antibody complexes. The BoNT/A toxin support is washed twice in 800 μL of Ringer's solution, pH 7.0 to remove any unbound antibodies. An optimally determined amount of a ¹²⁵-labeled FGFR3 is added to the BoNT/A toxin support and incubated at 37° C. for 20 minutes to permit binding of the labeled FGFR3 to any free BoNT/A toxins. The BoNT/A toxin support is washed twice in 800 μL of Ringer's solution, pH 7.0 to remove any unbound ¹²⁵-labeled FGFR3. The washed BoNT/A toxin support is added in 300 μL of Ringer's solution, pH 7.0, transferred to a glass scintillation tube, and the amount of radioactivity from the support measured using a gamma scintillation counter. The percent FGFR3 binding inhibition of a serum sample was calculated using the following formula: [1−(count of the sample/count of control)]×100. The negative control is a mixture of five serum samples taken from untreated individuals (never administered a BoNT/A therapy) and the radioactivity measured from this sample is used as a zero point reference to adjust for background. Assays are carried out in triplicates. A statistically significant decrease in the amount of radioactivity detected from the BoNT/A toxin support indicates the presence of neutralizing anti-BoNT/A antibodies in the serum sample. Conversely, a lack of any significant reduction in ¹²⁵-labeled FGFR3-BoNT/A complexes indicates the absence of neutralizing anti-BoNT/A antibodies.

### Example 15

### BoNT/A Immunoresistant Assay Using a BoNT/A Toxin, a FGFR3 Support and a Radioactive Detection Method

To express and purify a FGFR3, an expression procedure and an IMAC protein purification procedure as described in Example 13 is used. The amount of FGFR3 is adjusted to a protein concentration suitable for making a support matrix.

To make a FGFR3 support comprising a solid or insoluble material, 50 μL of Phosphate Buffered Saline, pH 7.2 (10 mM sodium phosphate, pH 7.2; 150 mM sodium chloride) containing approximately 1 μg FGFR3 is added to the wells of flexible polyvinyl chloride 96-well microplates (BD Biosciences, San Jose, Calif.) and allowed to bind at 4° C. for 18 hours. The plates are then washed five times with Phosphate Buffered Saline, pH 7.2 and then blocked with 1% (w/v) bovine serum albumin at 37° C. for 60 minutes. The plates are then washed five times with Phosphate Buffered Saline, pH 7.2 containing 0.1% (w/v) bovine serum albumin and stored in this solution at 4° C. until needed.

Preparation of a radiolabeled inactive BoNT/A toxin is described in Example 6.

To determine the amount of FGFR3 required to achieve saturation binding with a fixed amount of BoNT/A toxin, a receptor binding assay is conducted as described in Example 4 and as modified below with the receptor component comprising FGFR3s attached to a solid or insoluble material.

To determine the volume of a sample containing neutralizing anti-BoNT/A antibodies necessary to achieve saturation binding of a BoNT/A toxin to FGFR3, a BoNT/A receptor binding inhibition assay is conducted as described in Example 4 and as modified below with the receptor component comprising FGFR3s attached to a solid or insoluble material.

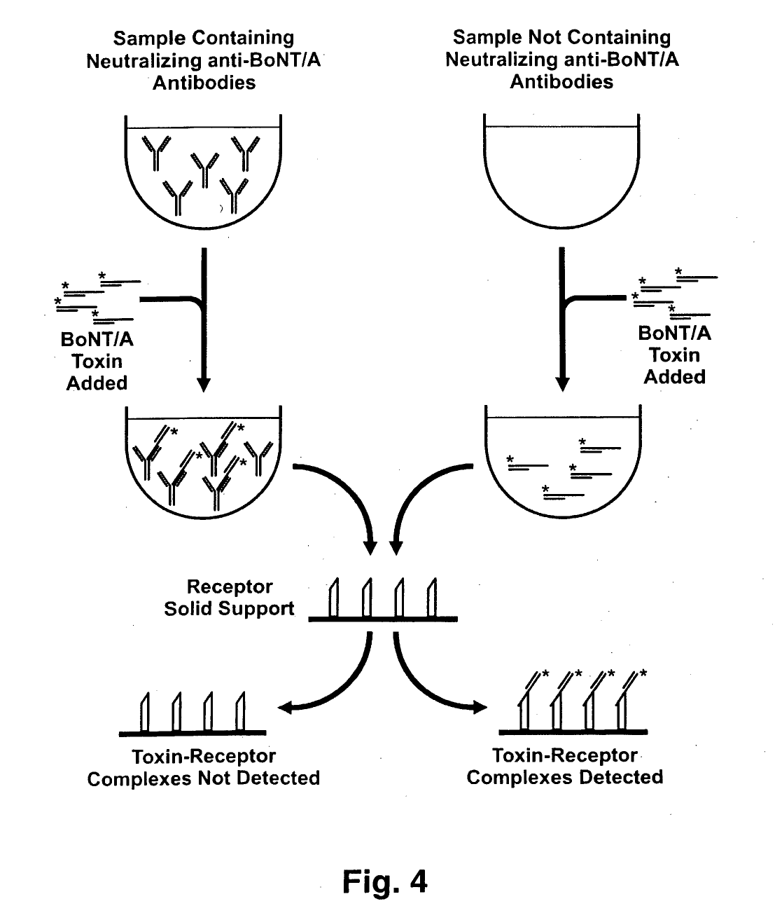

To conduct a BoNT/A Immunoresistant Assay (see, e.g., FIG. 4), an optimally determined volume of a serum sample and approximately 50,000 cpm of ¹²⁵Iodine-labeled inactive BoNT/A (about 1.0 ng) are added to 100 μL of Ringer's solution, pH 7.0 (120 mM sodium chloride, 2.5 mM potassium chloride, 2 mM calcium chloride, 4 mM magnesium chloride, 5 mM 2-amino-2-hydroxymethyl-1,3-propanediol hydrochloric acid (Tris-HCl, pH 7.0), 0.5% bovine serum albumin). This reaction mixture is incubated at 37° C. for 20 minutes in order to allow for the formation of any toxin-antibody complexes. The reaction mixture is then applied to a FGFR3 support comprising a solid or insoluble material and incubated at 37° C. for 20 minutes to allow the formation of any toxin-FGFR3 complexes. The FGFR3 support is washed twice in 800 μL of Ringer's solution, pH 7.0 to remove any toxin-antibody complexes and any unbound BoNT/A toxin. The washed FGFR3 support is added in 300 μL of Ringer's solution, pH 7.0, transferred to a glass scintillation tube, and the amount of radioactivity from the support measured using a gamma scintillation counter. The percent FGFR3 binding inhibition of a serum sample is calculated using the following formula: [1−(count of the sample/count of control)]×100. The negative control is a mixture of five serum samples taken from untreated individuals (never administered a BoNT/A therapy) and the radioactivity measured from this sample is used as a zero point reference to adjust for background. Assays are carried out in triplicates. A statistically significant decrease in the amount of radioactivity detected from the FGFR3 support indicates the presence of neutralizing anti-BoNT/A antibodies in the serum sample. Conversely, a lack of any significant reduction in ¹²⁵-labeled BoNT/A-FGFR3 complexes indicates the absence of neutralizing anti-BoNT/A antibodies.

Although the present invention has been described with reference to the disclosed embodiments, one skilled in the art will readily appreciate that the specific experiments disclosed are only illustrative of the present invention. Various modifications can be made without departing from the spirit of the present invention.

What is claimed:

1. A method of determining BoNT/A immunoresistance a mammal comprising the steps of:
a) adding a BoNT/A toxin and a BoNT/A receptor to a test sample; and
b) detecting the amount of toxin-receptor complexes formed in the test sample.

2. The method according to claim 1, wherein the mammal is a human.

3. The method according to claim 1, wherein the BoNT/A toxin is active BoNT/A toxin.

4. The method according to claim 1, wherein the BoNT/A toxin is a non-toxic BoNT/A toxin.

5. The method according to claim 1, wherein the BoNT/A toxin is a BoNT/A pharmaceutical composition.

6. The method according to claim 1, wherein the BoNT/A toxin comprising amino acids 449 to 1296 of SEQ ID NO: 1.

7. The method according to claim 1, wherein the BoNT/A toxin comprising amino acids 855 to 1296 of SEQ ID NO: 1.

8. The method according to claim 1, wherein the BoNT/A toxin is one or more peptides selected from the group consisting of amino acids 463 to 481 of SEQ ID NO: 1, 505 to 523 of SEQ ID NO: 1, 519 to 537 of SEQ ID NO: 1, 533 to 551 of SEQ ID NO: 1, 603 to 621 of SEQ ID NO: 1, 645 to 663 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1079 to 1097 of SEQ ID NO: 1, 1107 to 1125 of SEQ ID NO: 1, 1163 to 1181 of SEQ ID NO: 1, 1177 to 1195 of SEQ ID NO: 1, 1191 to 1209 of SEQ ID NO: 1, 1233 to 1251 of SEQ ID NO: 1 and 1275 to 1296 of SEQ ID NO: 1.

9. The method according to claim 1, wherein the BoNT/A toxin is one or more peptides selected from the group consisting of amino acids 533 to 551 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1107 to 1125 of SEQ ID NO: 1, 1163 to 1181 of SEQ ID NO: 1, 1191 to 1209 of SEQ ID NO: 1, 1233 to 1251 of SEQ ID NO: 1/1275 to 1296 of SEQ ID NO: 1.

10. The method according to claim 1, wherein the BoNT/A toxin is one or more peptides selected from the group consisting of amino acids 659 to 677 of SEQ ID NO: 1, 729 to 747 of SEQ ID NO: 1, 799 to 817 of SEQ ID NO: 1, 1065 to 1083 of SEQ ID NO: 1, 1163 to 1181 of SEQ ID NO: 1, and 1275 to 1296 of SEQ ID NO: 1.

11. The method to according claim 1, wherein the BoNT/A toxin is operably-linked to a compound selected from the group consisting of a radioisotope, a fluorescent compound, a phosphorescent compound, a chemiluminescent compound, and a bioluminescent compound.

12. The method to according claim 1, wherein the BoNT/A toxin is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

13. The method to according claim 1, wherein the BoNT/A toxin is attached to a solid support.

14. The method according to claim 1, wherein the BoNT/A receptor is a FGFR3.

15. The method according to claim 1, wherein the BoNT/A receptor is a FGFR3 selected from the group consisting of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35 and SEQ ID NO: 37.

16. The method according to claim 1, wherein the BoNT/A receptor is a mammalian tissue preparation selected from the group consisting of a synaptosome preparation, a synaptic vesicle preparation, a synaptic membrane preparation and a synaptic density preparation.

17. The method to according claim 1, wherein the BoNT/A receptor is operably-linked to a compound selected from the group consisting of a radioisotope, a fluorescent compound, a phosphorescent compound, a chemiluminescent compound, and a bioluminescent compound.

18. The method to according claim 1, wherein the BoNT/A receptor is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

19. The method to according claim 1, wherein the BoNT/A receptor is attached to a solid support.

20. The method to according claim 1, wherein the test sample is selected from the group consisting of mammalian blood, human blood, mammalian plasma, human plasma, mammalian serum and human serum.

21. The method to according claim 1, wherein the test sample is human serum.

22. The method to according claim 1, further comprising the step of:
a) adding a BoNT/A toxin and a BoNT/A receptor to a control sample;
b) detecting the amount of toxin-receptor complexes formed in the control sample; and
c) comparing the amount of toxin-receptor complexes formed in the test sample to the amount of toxin-receptor complexes formed in the control sample.

23. The method according to claim 22, wherein the control sample is obtained from an individual not exhibiting BoNT/A immunoresistance.

24. The method according to claim 22, wherein the control sample is obtained from an individual exhibiting BoNT/A immunoresistance.

25. A method of determining BoNT/A immunoresistance in a mammal comprising the steps of:
a) adding a BoNT/A toxin to a test sample;
b) contacting the toxin with a BoNT/A receptor; and
c) detecting the amount of toxin-receptor complexes formed in the test sample.

26. The method according to claim 25, wherein the mammal is a human.

27. The method according to claim 25, wherein the BoNT/A toxin is active BoNT/A toxin.

28. The method according to claim 25, wherein the BoNT/A toxin is a non-toxic BoNT/A toxin.

29. The method according to claim 25, wherein the BoNT/A toxin is a BoNT/A pharmaceutical composition.

30. The method according to claim 25, wherein the BoNT/A toxin comprising amino acids 449 to 1296 of SEQ ID NO: 1.

31. The method according to claim 25, wherein the BoNT/A toxin comprising amino acids 855 to 1296 of SEQ ID NO: 1.

32. The method according to claim 25, wherein the BoNT/A toxin is one or more peptides selected from the group consisting of amino acids 463 to 481 of SEQ ID NO: 1, 505 to 523 of SEQ ID NO: 1, 519 to 537 of SEQ ID NO: 1, 533 to 551 of SEQ ID NO: 1, 603 to 621 of SEQ ID NO: 1, 645 to 663 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1079 to 1097 of SEQ ID NO: 1, 1107 to 1125 of SEQ ID NO: 1, 1163 to 1181 of SEQ ID NO: 1, 1177 to 1195 of SEQ ID NO: 1, 1191 to 1209 of SEQ ID NO: 1, 1233 to 1251 of SEQ ID NO: 1 and 1275 to 1296 of SEQ ID NO: 1.

33. The method according to claim 25, wherein the BoNT/A toxin is one or more peptides selected from the group consisting of amino acids 533 to 551 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1107 to 1125 of SEQ ID NO: 1, 1163 to 1181 of SEQ ID NO: 1, 1191 to 1209 of SEQ ID NO: 1, 1233 to 1251 of SEQ ID NO: 1/1275 to 1296 of SEQ ID NO: 1.

34. The method according to claim 25, wherein the BoNT/A toxin is one or more peptides selected from the group consisting of amino acids 659 to 677 of SEQ ID NO: 1, 729 to 747 of SEQ ID NO: 1, 799 to 817 of SEQ ID NO: 1, 1065 to 1083 of SEQ ID NO: 1, 1163 to 1181 of SEQ ID NO: 1, and 1275 to 1296 of SEQ ID NO: 1.

35. The method to according claim 25, wherein the BoNT/A toxin is operably-linked to a compound selected from the group consisting of a radioisotope, a fluorescent compound, a phosphorescent compound, a chemiluminescent compound, and a bioluminescent compound.

36. The method to according claim 25, wherein the BoNT/A toxin is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

37. The method to according claim 25, wherein the BoNT/A toxin is attached to a solid support.

38. The method according to claim 25, wherein the BoNT/A receptor is a FGFR3.

39. The method according to claim 25, wherein the BoNT/A receptor is a FGFR3 selected from the group consisting of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35 and SEQ ID NO: 37.

40. The method according to claim 25, wherein the BoNT/A receptor is a mammalian tissue preparation selected from the group consisting of a synaptosome preparation, a synaptic vesicle preparation, a synaptic membrane preparation and a synaptic density preparation.

41. The method to according claim 25, wherein the BoNT/A receptor is operably-linked to a compound selected from the group consisting of a radioisotope, a fluorescent compound, a phosphorescent compound, a chemiluminescent compound, and a bioluminescent compound.

42. The method to according claim 25, wherein the BoNT/A receptor is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

43. The method to according claim 25, wherein the BoNT/A receptor is attached to a solid support.

44. The method to according claim 25, wherein the test sample is selected from the group consisting of mammalian blood, human blood, mammalian plasma, human plasma, mammalian serum and human serum.

45. The method to according claim 25, further comprising the step of:
a) adding a BoNT/A toxin to a control sample;
b) contacting the toxin with a BoNT/A receptor;
c) detecting the amount of toxin-receptor complexes formed in the control sample; and
d) comparing the amount of toxin-receptor complexes formed in the test sample relative to the amount of toxin-receptor complexes formed in the control sample.

46. The method according to claim 45, wherein the control sample is obtained from an individual not exhibiting BoNT/A immunoresistance.

47. The method according to claim 45, wherein the control sample is obtained from an individual exhibiting BoNT/A immunoresistance.

48. A method of determining BoNT/A immunoresistance in a mammal comprising the steps of:
a) adding a BoNT/A toxin and a BoNT/A receptor to a test sample under conditions in which a neutralizing anti-BoNT/A antibody can bind the toxin to form a toxin-antibody complex and the toxin can bind the receptor to form a toxin-receptor complex; and
b) detecting the presence or absence of one or more the toxin-receptor complexes; wherein the presence of the toxin-receptor complexes indicates the lack of BoNT/A immunoresistance and the absence of the toxin-receptor complexes indicates BoNT/A immunoresistance.

49. The method according to claim 48, wherein the mammal is a human.

50. The method according to claim 48, wherein the BoNT/A toxin is active BoNT/A toxin.

51. The method according to claim 48, wherein the BoNT/A toxin is a non-toxic BoNT/A toxin.

52. The method according to claim 48, wherein the BoNT/A toxin is a BoNT/A pharmaceutical composition.

53. The method according to claim 48, wherein the BoNT/A toxin comprising amino acids 449 to 1296 of SEQ ID NO: 1.

54. The method according to claim 48, wherein the BoNT/A toxin comprising amino acids 855 to 1296 of SEQ ID NO: 1.

55. The method according to claim 48, wherein the BoNT/A toxin is one or more peptides selected from the group consisting of amino acids 463 to 481 of SEQ ID NO: 1, 505 to 523 of SEQ ID NO: 1, 519 to 537 of SEQ ID NO: 1, 533 to 551 of SEQ ID NO: 1, 603 to 621 of SEQ ID NO: 1, 645 to 663 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1079 to 1097 of SEQ ID NO: 1, 1107 to 1125 of SEQ ID NO: 1, 1163 to 1181 of SEQ ID NO: 1, 1177 to 1195 of SEQ ID NO: 1, 1191 to 1209 of SEQ ID NO: 1, 1233 to 1251 of SEQ ID NO: 1 and 1275 to 1296 of SEQ ID NO: 1.

56. The method according to claim 48, wherein the BoNT/A toxin is one or more peptides selected from the group consisting of amino acids 533 to 551 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1107 to 1125 of SEQ ID NO: 1, 1163 to 1181 of SEQ ID NO: 1, 1191 to 1209 of SEQ ID NO: 1, 1233 to 1251 of SEQ ID NO: 1/1275 to 1296 of SEQ ID NO: 1.

57. The method according to claim 48, wherein the BoNT/A toxin is one or more peptides selected from the group consisting of amino acids 659 to 677 of SEQ ID NO: 1, 729 to 747 of SEQ ID NO: 1, 799 to 817 of SEQ ID NO: 1, 1065 to 1083 of SEQ ID NO: 1, 1163 to 1181 of SEQ ID NO: 1, and 1275 to 1296 of SEQ ID NO: 1.

58. The method to according claim 48, wherein the BoNT/A toxin is operably-linked to a compound selected from the group consisting of a radioisotope, a fluorescent compound, a phosphorescent compound, a chemiluminescent compound, and a bioluminescent compound.

59. The method to according claim 48, wherein the BoNT/A toxin is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

60. The method to according claim 48, wherein the BoNT/A toxin is attached to a solid support.

61. The method according to claim 48, wherein the BoNT/A receptor is a FGFR3.

62. The method according to claim 48, wherein the BoNT/A receptor is a FGFR3 selected from the group consisting of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35 and SEQ ID NO: 37.

63. The method according to claim 48, wherein the BoNT/A receptor is a mammalian tissue preparation selected from the group consisting of a synaptosome preparation, a synaptic vesicle preparation, a synaptic membrane preparation and a synaptic density preparation.

64. The method to according claim 48, wherein the BoNT/A receptor is operably-linked to a compound selected from the group consisting of a radioisotope, a fluorescent compound, a phosphorescent compound, a chemiluminescent compound, and a bioluminescent compound.

65. The method to according claim 48, wherein the BoNT/A receptor is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

66. The method to according claim 48, wherein the BoNT/A receptor is attached to a solid support.

67. The method to according claim 48, wherein the test sample is selected from the group consisting of mammalian blood, human blood, mammalian plasma, human plasma, mammalian serum and human serum.

68. The method to according claim 48, wherein the test sample is human serum.

69. A method of determining BoNT/A immunoresistance in a mammal comprising the steps of:
a) adding a BoNT/A toxin to a test sample under conditions in which a neutralizing anti-BoNT/A antibody can bind the toxin to form a toxin-antibody complex;
b) adding a BoNT/A receptor to the test sample under conditions in which the toxin can bind the receptor to form a toxin-receptor complex; and
c) detecting the presence or absence of one or more the toxin-receptor complexes; wherein the presence of the toxin-receptor complexes indicates the lack of BoNT/A immunoresistance and the absence of the toxin-receptor complexes indicates BoNT/A immunoresistance.

70. The method according to claim 69, wherein the mammal is a human.

71. The method according to claim 69, wherein the BoNT/A toxin is active BoNT/A toxin.

72. The method according to claim 69, wherein the BoNT/A toxin is a non-toxic BoNT/A toxin.

73. The method according to claim 69, wherein the BoNT/A toxin is a BoNT/A pharmaceutical composition.

74. The method according to claim 69, wherein the BoNT/A toxin comprising amino acids 449 to 1296 of SEQ ID NO: 1.

75. The method according to claim 69, wherein the BoNT/A toxin comprising amino acids 855 to 1296 of SEQ ID NO: 1.

76. The method according to claim 69, wherein the BoNT/A toxin is one or more peptides selected from the group consisting of amino acids 463 to 481 of SEQ ID NO: 1, 505 to 523 of SEQ ID NO: 1, 519 to 537 of SEQ ID NO: 1, 533 to 551 of SEQ ID NO: 1, 603 to 621 of SEQ ID NO: 1, 645 to 663 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1079 to 1097 of SEQ ID NO: 1, 1107 to 1125 of SEQ ID NO: 1, 1163 to 1181 of SEQ ID NO: 1, 1177 to 1195 of SEQ ID NO: 1, 1191 to 1209 of SEQ ID NO: 1, 1233 to 1251 of SEQ ID NO:1 and 1275 to 1296 of SEQ ID NO: 1.

77. The method according to claim 69, wherein the BoNT/A toxin is one or more peptides selected from the group consisting of amino acids 533 to 551 of SEQ ID NO: 1, 659 to 677 of SEQ ID NO: 1, 701 to 719 of SEQ ID NO: 1, 729 to 747 of SEQ ID NO: 1, 757 to 775 of SEQ ID NO: 1, 799 to 817 of SEQ ID NO: 1, 1065 to 1083 of SEQ ID NO: 1, 1107 to 1125 of SEQ ID NO: 1, 1163 to 1181 of SEQ ID NO: 1, 1191 to 1209 of SEQ ID NO: 1, 1233 to 1251 of SEQ ID NO: 1/1275 to 1296 of SEQ ID NO: 1.

78. The method according to claim 69, wherein the BoNT/A toxin is one or more peptides selected from the group consisting of amino acids 659 to 677 of SEQ ID NO: 1, 729 to 747 of SEQ ID NO: 1, 799 to 817 of SEQ ID NO: 1, 1065 to 1083 of SEQ ID NO: 1, 1163 to 1181 of SEQ ID NO: 1, and 1275 to 1296 of SEQ ID NO: 1.

79. The method to according claim 69, wherein the BoNT/A toxin is operably-linked to a compound selected from the group consisting of a radioisotope, a fluorescent compound, a phosphorescent compound, a chemiluminescent compound, and a bioluminescent compound.

80. The method to according claim 69, wherein the BoNT/A toxin is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

81. The method to according claim 69, wherein the BoNT/A toxin is attached to a solid support.

82. The method according to claim 69, wherein the BoNT/A receptor is a FGFR3.

83. The method according to claim 69, wherein the BoNT/A receptor is a FGFR3 selected from the group consisting of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35 and SEQ ID NO: 37.

84. The method according to claim 69, wherein the BoNT/A receptor is a mammalian tissue preparation selected from the group consisting of a synaptosome preparation, a synaptic vesicle preparation, a synaptic membrane preparation and a synaptic density preparation.

85. The method to according claim 69, wherein the BoNT/A receptor is operably-linked to a compound selected from the group consisting of a radioisotope, a fluorescent compound, a phosphorescent compound, a chemiluminescent compound, and a bioluminescent compound.

86. The method to according claim 69, wherein the BoNT/A receptor is a fusion protein operably-linked to a peptide selected from the group consisting of a peptide capable of producing fluorescence, a peptide capable of producing chemiluminescence, a peptide capable of producing bioluminescence and a peptide capable of producing a chromogenic compound.

87. The method to according claim 69, wherein the BoNT/A receptor is attached to a solid support.

88. The method to according claim 69, wherein the test sample is selected from the group consisting of mammalian blood, human blood, mammalian plasma, human plasma, mammalian serum and human serum.

89. The method to according claim 69, wherein the test sample is human serum.

90. A method of determining BoNT/A immunoresistance a mammal comprising the steps of:
a) adding a BoNT/A toxin and a BoNT/A receptor to a test sample; and
b) detecting the amount of said free receptor in the test sample.

91. The method to according claim 90, further comprising the step of:
a) adding a BoNT/A toxin and a BoNT/A receptor to a control sample;
b) detecting the amount of said free receptor in the control sample; and
c) comparing the amount of free receptor in the test sample to the amount of free receptor in the control sample.

92. A method of determining BoNT/A immunoresistance in a mammal comprising the steps of:
a) adding a BoNT/A toxin to a test sample;
b) contacting the toxin with a BoNT/A receptor; and
c) detecting the amount of said free receptor in the test sample.

93. The method to according claim 92, further comprising the step of:
a) adding a BoNT/A toxin to a control sample;
b) contacting the toxin with a BoNT/A receptor;
c) detecting the amount of said free receptor in the control sample; and
d) comparing the amount of free receptor in the test sample relative to the amount of free receptor in the control sample.

94. A method of determining BoNT/A immunoresistance in a mammal comprising the steps of:
a) adding a BoNT/A toxin and a BoNT/A receptor to a test sample under conditions in which a neutralizing anti-BoNT/A antibody can bind the toxin to form a toxin-antibody complex and the toxin can bind the receptor to form a toxin-receptor complex; and
b) detecting the presence or absence of free receptor; wherein the presence of the free receptor indicates BoNT/A immunoresistance and the absence of the free receptor indicates the lack BoNT/A immunoresistance.

95. A method of determining BoNT/A immunoresistance in a mammal comprising the steps of:
a) adding a BoNT/A toxin to a test sample under conditions in which a neutralizing anti-BoNT/A antibody can bind the toxin to form a toxin-antibody complex;
b) adding a BoNT/A receptor to the test sample under conditions in which the toxin can bind the receptor to form a toxin-receptor complex; and
c) detecting the presence or absence of free receptor; wherein the presence of the free receptor indicates BoNT/A immunoresistance and the absence of the free receptor indicates the lack of BoNT/A immunoresistance.

96. A method of determining BoNT/A immunoresistance a mammal comprising the steps of:
a) adding a BoNT/A toxin and a BoNT/A receptor to a test sample; and
b) detecting the amount of toxin-antibody complexes formed in the test sample.

97. The method to according claim 96, further comprising the step of:
a) adding a BoNT/A toxin and a BoNT/A receptor to a control sample;
b) detecting the amount of toxin-antibody complexes formed in the control sample; and
c) comparing the amount of toxin-antibody complexes formed in the test sample to the amount of toxin-antibody complexes formed in the control sample.

98. A method of determining BoNT/A immunoresistance in a mammal comprising the steps of:
a) adding a BoNT/A toxin to a test sample;
b) contacting the toxin with a BoNT/A receptor; and
c) detecting the amount of toxin-antibody complexes formed in the test sample.

99. The method to according claim 98, further comprising the step of:
a) adding a BoNT/A toxin to a control sample;
b) contacting the toxin with a BoNT/A receptor;
c) detecting the amount of toxin-antibody complexes formed in the control sample; and
d) comparing the amount of toxin-antibody complexes formed in the test sample relative to the amount of toxin-antibody complexes formed in the control sample.

100. A method of determining BoNT/A immunoresistance in a mammal comprising the steps of:
a) adding a BoNT/A toxin and a BoNT/A receptor to a test sample under conditions in which a neutralizing anti-BoNT/A antibody can bind the toxin to form a toxin-antibody complex and the toxin can bind the receptor to form a toxin-receptor complex; and
b) detecting the presence or absence of toxin-antibody complexes; wherein the presence of the toxin-antibody complexes indicate BoNT/A immunoresistance and the absence of the toxin-antibody complexes indicate the lack BoNT/A immunoresistance.

101. A method of determining BoNT/A immunoresistance in a mammal comprising the steps of:
a) adding a BoNT/A toxin to a test sample under conditions in which a neutralizing anti-BoNT/A antibody can bind the toxin to form a toxin-antibody complex;
b) adding a BoNT/A receptor to the test sample under conditions in which the toxin can bind the receptor to form a toxin-receptor complex; and
c) detecting the presence or absence of toxin-antibody complexes; wherein the presence of the toxin-antibody complexes indicate BoNT/A immunoresistance and the absence of the toxin-antibody complexes indicate the lack BoNT/A immunoresistance.
